SEQUENCE LISTING (74 sequences)

SEQ ID NO: 1 - DNA, 47 bp, Artificial
  EC1 5′ primer
cacgcggatgaaatcgataagctcgagccccccggaatcgcgggcac

SEQ ID NO: 2 - DNA, 38 bp, Artificial
  EC1 3′ primer
ccggactagtgacctcttggttattcgggggacacacc

SEQ ID NO: 3 - DNA, 34 bp, Artificial
  EC2 5′ primer
ccgggtcgactgcccctacaactacctgtctacg

SEQ ID NO: 4 - DNA, 58 bp, Artificial
  EC2 3′ primer
ccggactagtttacttgtcatcgtcgtccttgtagtccccactgtggagcagggcctg

SEQ ID NO: 5 - DNA, 34 bp, Artificial
  EC3 5′ primer
ccgggtcgactgctttgtacacactgtaccttgg

SEQ ID NO: 6 - DNA, 58 bp, Artificial
  EC3 3′ primer
ccggactagtttacttgtcatcgtcgtccttgtagtccgggctggctctctgctctgc

SEQ ID NO: 7 - DNA, 35 bp, Artificial
  IC1 5′ primer
ccggctcgagtatacgatgcgtaggctgctgcagg

SEQ ID NO: 8 - DNA, 34 bp, Artificial
  IC1 3′ primer
ccggactagtagccagtggagatctggggggccc

SEQ ID NO: 9 - DNA, 32 bp, Artificial
  IC2 5′ primer
ccggctcgagggtgacctggtagacgctgaag

SEQ ID NO: 10 - DNA, 32 bp, Artificial
  IC2 3′ primer
ccggactagttacaggtacatccaggcctagg

SEQ ID NO: 11 - protein, 9 aa, Homo sapiens
ALCRWGLLL

SEQ ID NO: 12 - protein, 9 aa, Homo sapiens
HLYQGCQVV

SEQ ID NO: 13 - protein, 15 aa, Homo sapiens
LTYLPTNASLSFLQD

SEQ ID NO: 14 - protein, 9 aa, Homo sapiens
TYLPTNASL

SEQ ID NO: 15 - protein, 9 aa, Homo sapiens
QLFEDNYAL

SEQ ID NO: 16 - protein, 9 aa, Homo sapiens
KIGFSLAFL

SEQ ID NO: 17 - protein, 18 aa, Homo sapiens
KIFGSLAFLPESFDGDPA

SEQ ID NO: 18 - protein, 441 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 19 - protein, 416 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 20 - protein, 390 aa, Listeria monocytogenes
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 21 - DNA, 1170 bp, Listeria monocytogenes
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 22 - protein, 32 aa, Listeria monocytogenes
KENSISSMAPPASPPASPKTPIEKKHADEIDK

SEQ ID NO: 23 - protein, 19 aa, Listeria monocytogenes
KENSISSMAPPASPPASPK

SEQ ID NO: 24 - DNA, 28 bp, Artificial
  prfA primer-2
gggggtcgaccagctcttcttggtgaag

SEQ ID NO: 25 - protein, 14 aa, Listeria monocytogenes
KTEEQPSEVNTGPR

SEQ ID NO: 26 - protein, 28 aa, Listeria monocytogenes
KASVTDTSEGDLDSSMQSADESTPQPLK

SEQ ID NO: 27 - protein, 20 aa, Listeria monocytogenes
KNEEVNASDFPPPPTDEELR

SEQ ID NO: 28 - protein, 33 aa, Listeria monocytogenes
RGGIPTSEEFSSLNSGDFTDDENSETTEEEIDR

SEQ ID NO: 29 - protein, 19 aa, Listeria seeligeri
RSEVTISPAETPESPPATP

SEQ ID NO: 30 - protein, 17 aa, Streptococcus pyogenes
KQNTASTETTTTNEQPK

SEQ ID NO: 31 - protein, 17 aa, Streptococcus equisimilis
KQNTANTETTTTNEQPK

SEQ ID NO: 32 - protein, 1259 aa, Rattus rattus
MIIMELAAWCRWGFLLALLPPGIAGTQVCTGTDMKLRLPASPETHLDMLRHLYQGCQVVQGNLELTYVPANASLSFLQDIQEVQGYMLIAHNQVKRVPLQRLRIVRGTQLFEDKYALAVLDNRDPQDNVAASTPGRTPEGLRELQLRSLTEILKGGVLIRGNPQLCYQDMVLWKDVFRKNNQLAPVDIDTNRSRACPPCAPACKDNHCWGESPEDCQILTGTICTSGCARCKGRLPTDCCHEQCAAGCTGPKHSDCLACLHFNHSGICELHCPALVTYNTDTFESMHNPEGRYTFGASCVTTCPYNYLSTEVGSCTLVCPPNNQEVTAEDGTQRCEKCSKPCARVCYGLGMEHLRGARAITSDNVQEFDGCKKIFGSLAFLPESFDGDPSSGIAPLRPEQLQVFETLEEITGYLYISAWPDSLRDLSVFQNLRIIRGRILHDGAYSLTLQGLGIHSLGLRSLRELGSGLALIHRNAHLCFVHTVPWDQLFRNPHQALLHSGNRPEEDCGLEGLVCNSLCAHGHCWGPGPTQCVNCSHFLRGQECVEECRVWKGLPREYVSDKRCLPCHPECQPQNSSETCFGSEADQCAACAHYKDSSSCVARCPSGVKPDLSYMPIWKYPDEEGICQPCPINCTHSCVDLDERGCPAEQRASPVTFIIATVEGVLLFLILVVVVGILIKRRRQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVREHRGRLGSQDLLNWCVQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPSSPMDSTFYRSLLEDDDMGDLVDAEEYLVPQQGFFSPDPTPGTGSTAHRRHRSSSTRSGGGELTLGLEPSEEGPPRSPLAPSEGAGSDVFDGDLAMGVTKGLQSLSPHDLSPLQRYSEDPTLPLPPETDGYVAPLACSPQPEYVNQSEVQPQPPLTPEGPLPPVRPAGATLERPKTLSPGKNGVVKDVFAFGGAVENPEYLVPREGTASPPHPSPAFSPAFDNLYYWDQNSSEQGPPPSNFEGTPTAENPEYLGLDVPV

SEQ ID NO: 33 - protein, 1255 aa, Homo sapiens [sequence omitted]

SEQ ID NO: 34 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 35 - protein, 9 aa, Homo sapiens
PLQPEQLQV

SEQ ID NO: 36 - protein, 9 aa, Homo sapiens
TLEEITGYL

SEQ ID NO: 37 - protein, 9 aa, Homo sapiens
ILHNGAYSL

SEQ ID NO: 38 - protein, 9 aa, Homo sapiens
ALIHHNTHL

SEQ ID NO: 39 - protein, 15 aa, Homo sapiens
KPDLSYMPIWKFPDE

SEQ ID NO: 40 - protein, 9 aa, Homo sapiens
PLTSIISAV

SEQ ID NO: 41 - protein, 9 aa, Homo sapiens
IISAVVGIL

SEQ ID NO: 42 - protein, 16 aa, Homo sapiens
RRLLQETELVEPLTPS

SEQ ID NO: 43 - protein, 9 aa, Homo sapiens
RLLQETELV

SEQ ID NO: 44 - protein, 9 aa, Homo sapiens
VLRENTSPK

SEQ ID NO: 45 - protein, 19 aa, Homo sapiens
KEILDEAYVMAGVGSPYVS

SEQ ID NO: 46 - protein, 10 aa, Homo sapiens
VMAGVGSPYV

SEQ ID NO: 47 - protein, 13 aa, Homo sapiens
GSPYVSRLLGICL

SEQ ID NO: 48 - protein, 13 aa, Homo sapiens
SPYVSRLLGICLT

SEQ ID NO: 49 - protein, 9 aa, Homo sapiens
PYVSRLLGI

SEQ ID NO: 50 - protein, 10 aa, Homo sapiens
LLGICLTSTV

SEQ ID NO: 51 - protein, 9 aa, Homo sapiens
CLTSTVQLV

SEQ ID NO: 52 - protein, 9 aa, Homo sapiens
QLMPYGCLL

SEQ ID NO: 53 - protein, 15 aa, Homo sapiens
LLNWCMQIAKGMSYL

SEQ ID NO: 54 - protein, 8 aa, Homo sapiens
YLEDVRLV

SEQ ID NO: 55 - protein, 9 aa, Homo sapiens
VLVKSPNHV

SEQ ID NO: 56 - protein, 17 aa, Homo sapiens
KVPIKWMALESILRRRF

SEQ ID NO: 57 - protein, 10 aa, Homo sapiens
YMIMVKCWMI

SEQ ID NO: 58 - protein, 9 aa, Homo sapiens
ELVSEFSRM

SEQ ID NO: 59 - protein, 14 aa, Homo sapiens
ELVSEFSRMARDPQ

SEQ ID NO: 60 - protein, 10 aa, Homo sapiens
YLVPQQGFFC

SEQ ID NO: 61 - DNA, 31 bp, Artificial
  pHly primer-1
ggggtctagacctcctttgattagtatattc

SEQ ID NO: 62 - DNA, 45 bp, Artificial
  pHly primer-2
atcttcgctatctgtcgccgcggcgcgtgcttcagtttgttgcgc

SEQ ID NO: 63 - DNA, 45 bp, Artificial
  ActA primer-1
gcgcaacaaactgaagcagcggccgcggcgacagatagcgaagat

SEQ ID NO: 64 - DNA, 42 bp, Artificial
  ActA primer-2
tgtaggtgtatctccatgctcgagagctaggcgatcaatttc

SEQ ID NO: 65 - DNA, 42 bp, Artificial
  E7 primer-1
ggaattgatcgcctagctctcgagcatggagatacacctaca

SEQ ID NO: 66 - DNA, 42 bp, Artificial
  E7 primer-2
aaacggatttatttagatcccgggttatggtttctgagaaca

SEQ ID NO: 67 - DNA, 42 bp, Artificial
  prfA primer-1
tgttctcagaaaccataacccgggatctaaataaatccgttt

SEQ ID NO: 68 - protein, 10 aa, Homo sapiens
GSGAFGTVYK

SEQ ID NO: 69 - protein, 9 aa, Homo sapiens
AFGTVYKGI

SEQ ID NO: 70 - protein, 9 aa, Homo sapiens
PYVSRLLGI

SEQ ID NO: 71 - protein, 9 aa, Homo sapiens
TSPKANKEI

SEQ ID NO: 72 - protein, 10 aa, Homo sapiens
RPRFRELVSE

SEQ ID NO: 73 - protein, 9 aa, Homo sapiens
STVQLVTQL

SEQ ID NO: 74 - protein, 10 aa, Homo sapiens
KITDFGLARL

(19) United States
(12) Patent Application Publication
(10) Pub. No.: US 2008/0124354 A1
(43) Pub. Date: May 29, 2008

(54) Methods for administering tumor vaccines

(76) Inventors: Yvonne Paterson, Philadelphia, PA (US); Reshma Singh, New Haven, CT (US); Paulo Maciag, (US)

Correspondence Address: Pearl Cohen Zedek Latzer, LLP, 1500 Broadway, 12th Floor, New York, NY, 10036, US

(21) Appl. No.: 11/822,870

(22) Filed: Jul. 10, 2007

Related U.S. Application Data
(60) Provisional application No. 60/819,360, filed on Jul. 10, 2006.

(51) Int. Cl. A61K 39/00; A61K 39/02; A61K 38/02; C07K 7/06; C07H 21/04; A61K 31/711; A61P 35/00

(52) U.S. Cl. 424/198.1; 424/184.1; 424/277.1; 424/234.1; 514/2; 530/328; 536/23.1; 514/44

(57) ABSTRACT

The present invention provides methods of inducing an immune response in a subject against a protein of interest and treating and suppressing a formation of a tumor comprising a tumor-associated protein, comprising sequential administration of fragments of the protein. The present invention also provides Her-2 protein fragments, recombinant peptides comprising same, nucleotide molecules encoding same, recombinant vaccine vectors and immunogenic compositions comprising same, and methods for inducing immune response and treating cancer, comprising same.

### CROSS REFERENCE TO RELATED APPLICATION

This application priority of U.S. Provisional Application Ser. No. 60/819,360, filed Jul. 10, 2006. This application is hereby incorporated in its entirety by reference herein.

### STATEMENT REGARDING FEDERALLY SPONSORED RESEARCH OR DEVELOPMENT

The invention described herein was supported in whole or in part by grants from The Department of Defense (W81XWH-04-1-0338). The government has certain rights in the invention.

### FIELD OF INVENTION

The present invention provides methods of inducing an immune response in a subject against a protein of interest and treating and suppressing a formation of a tumor comprising a tumor-associated protein, comprising sequential administration of fragments of the protein. The present invention also provides Her-2 protein fragments, recombinant peptides comprising same, nucleotide molecules encoding same, recombinant vaccine vectors and immunogenic compositions comprising same, and methods for inducing immune response and treating cancer, comprising same.

### BACKGROUND OF THE INVENTION

Her-2/neu (referred to henceforth as “Her-2”) is a 185 kDa glycoprotein that is a member of the epidermal growth factor receptor (EGFR) family of tyrosine kinases, and consists of an extracellular domain, a transmembrane domain, and an intracellular domain which is known to be involved in cellular signaling (Bargmann C I et al, Nature 319: 226, 1986; King C R et al, Science 229: 974, 1985). It is overexpressed in 25 to 40% of all breast cancers and is also overexpressed in many cancers of the ovaries, lung, pancreas, and gastrointestinal tract. The overexpression of Her-2 is associated with uncontrolled cell growth and signaling, both of which contribute to the development of tumors. Patients with cancers that over-express Her-2 exhibit tolerance even with detectable humoral, CD8⁺ T cell, and CD4⁺ T cell responses directed against Her-2.

Methods of inducing immune responses against oncoproteins such as Her-2, and of treating and suppressing formation of tumors that express such oncoproteins, are urgently needed in the art.

### SUMMARY OF THE INVENTION

The present invention provides methods of inducing an immune response in a subject against a protein of interest and treating and suppressing a formation of a tumor comprising a tumor-associated protein, comprising sequential administration of fragments of the protein. The present invention also provides Her-2 protein fragments, recombinant peptides comprising same, nucleotide molecules encoding same, recombinant vaccine vectors and immunogenic compositions comprising same, and methods for inducing immune response and treating cancer, comprising same.

In one embodiment, the present invention provides a method of inducing an immune response in a subject against a protein of interest, the method comprising the steps of: (A) administering to the subject a vaccine comprising a first recombinant peptide, the first recombinant peptide comprising a first fragment of the protein of interest; and (B) after the conclusion of step (A), administering to the subject a vaccine comprising a second recombinant peptide, the second recombinant peptide comprising a second fragment of the protein of interest, thereby inducing an immune response in a subject against a protein of interest. In another embodiment, the protein of interest is a tumor-associated protein. In another embodiment, the protein of interest is an endogenous growth factor. In another embodiment, the protein of interest is a vaso-active peptide or protein. In another embodiment, the protein of interest is any other protein known in the art. Each possibility represents a separate embodiment of the present invention.

In another embodiment, the present invention provides a method of treating a tumor expressing a tumor-associated protein in a subject, comprising the steps of: (A) administering to the subject a vaccine comprising a first recombinant peptide, the first recombinant peptide comprising a first fragment of the tumor-associated protein; and subsequent to step (A), administering to the subject a vaccine comprising a second recombinant peptide, the second recombinant peptide comprising a second fragment of the tumor-associated protein, thereby treating a tumor expressing a tumor-associated protein in a subject.

In another embodiment, the present invention provides a method of suppressing a formation of a tumor expressing a tumor-associated protein in a subject, comprising the steps of: (A) administering to the subject a vaccine comprising a first recombinant peptide, the first recombinant peptide comprising a first fragment of the tumor-associated protein; and (B) subsequent to step (A), administering to the subject a vaccine comprising a second recombinant peptide, the second recombinant peptide comprising a second fragment of the tumor-associated protein, thereby suppressing a formation of a tumor expressing a tumor-associated protein in a subject.

In another embodiment, the present invention provides a method of treating a tumor expressing a Her-2 protein in a subject, comprising the steps of:
(A) administering to the subject a first recombinant peptide, the first recombinant peptide comprising a first fragment of the Her-2 protein, wherein the first fragment is selected from: (1) a first intracellular fragment of the Her-2 protein, comprising the kinase domain of the Her-2 protein or a fragment of the kinase domain; (2) a second intracellular fragment of the Her-2 protein, wherein the second intracellular fragment either does not overlap with the first intracellular fragment or overlaps less than 20% with the first intracellular fragment; (3) a first extracellular fragment of the Her-2 protein, comprising the N-terminal half of the extracellular domain of the Her-2 protein or a fragment of the N-terminal half; and (4) a second extracellular fragment of the Her-2 protein, comprising the middle third of the extracellular domain of the Her-2 protein or a fragment of the middle third; and
(B) subsequent to step (A), administering to the subject a second recombinant peptide, the second recombinant peptide comprising a second fragment of the Her-2 protein, wherein the second fragment is selected from above fragments 1-4, but different from the first fragment, thereby treating a tumor expressing a Her-2 protein in a subject.

In another embodiment, the present invention provides a method of suppressing a formation of a tumor expressing a Her-2 protein in a subject, comprising the steps of:
A. administering to the subject a first recombinant peptide, the first recombinant peptide comprising a first fragment of the Her-2 protein, wherein the first fragment is selected from: (1) a first intracellular fragment of the Her-2 protein, comprising the kinase domain of the Her-2 protein or a fragment of the kinase domain; (2) a second intracellular fragment of the Her-2 protein, wherein the second intracellular fragment either does not overlap with the first intracellular fragment or overlaps less than 20% with the first intracellular fragment; (3) a first extracellular fragment of the Her-2 protein, comprising the N-terminal half of the extracellular domain of the Her-2 protein or a fragment of the N-terminal half; and (4) a second extracellular fragment of the Her-2 protein, comprising the middle third of the extracellular domain of the Her-2 protein or a fragment of the middle third; and
(B) subsequent to step (A), administering to the subject a second recombinant peptide, the second recombinant peptide comprising a second fragment of the Her-2 protein, wherein the second fragment is selected from above fragments 1-4, but different from the first fragment, thereby suppressing a formation of a tumor expressing a Her-2 protein in a subject.

In another embodiment, the present invention provides a recombinant peptide comprising a fragment of a Her-2 protein. In another embodiment, the fragment is ALCRWGLLL (SEQ ID No: 11). In another embodiment, the fragment is HLYQGCQVV (SEQ ID No: 12). In another embodiment, the fragment is LTYLPTNASLSFLQD (SEQ ID No: 13). In another embodiment, the fragment is TYLPTNASL (SEQ ID No: 14). In another embodiment, the fragment is QLFEDNYAL (SEQ ID No: 15). In another embodiment, the fragment is KIGFSLAFL (SEQ ID No: 16). In another embodiment, the fragment is KIFGSLAFLPESFDGDPA (SEQ ID No: 17). In another embodiment, the fragment is PLQPEQLQV (SEQ ID No: 35). In another embodiment, the fragment is TLEEITGYL (SEQ ID No: 36). In another embodiment, the fragment is ILHNGAYSL (SEQ ID No: 37). In another embodiment, the fragment is ALIHHNTHL (SEQ ID No: 38). In another embodiment, the fragment is KPDLSYMPIWKFPDE (SEQ ID No: 39). In another embodiment, the fragment is PLTSIISAV (SEQ ID No: 40). In another embodiment, the fragment is IISAVVGIL (SEQ ID No: 41). In another embodiment, the fragment is RRLLQETELVEPLTPS (SEQ ID No: 42). In another embodiment, the fragment is RLLQETELV (SEQ ID No: 43). In another embodiment, the fragment is VLRENTSPK (SEQ ID No: 44). In another embodiment, the fragment is KEILDEAYVMAGVGSPYVS (SEQ ID No: 45). In another embodiment, the fragment is VMAGVGSPYV (SEQ ID No: 46). In another embodiment, the fragment is GSPYVSRLLGICL (SEQ ID No: 47). In another embodiment, the fragment is SPYVSRLLGICLT (SEQ ID No: 48). In another embodiment, the fragment is PYVSRLLGI (SEQ ID No: 49). In another embodiment, the fragment is LLGICLTSTV (SEQ ID No: 50). In another embodiment, the fragment is CLTSTVQLV (SEQ ID No: 51). In another embodiment, the fragment is QLMPYGCLL (SEQ ID No: 52). In another embodiment, the fragment is LLNWCMQIAKGMSYL (SEQ ID No: 53). In another embodiment, the fragment is YLEDVRLV (SEQ ID No: 54). In another embodiment, the fragment is VLVKSPNHV (SEQ ID No: 55). In another embodiment, the fragment is KVPIKWMALESILRRRF (SEQ ID No: 56). In another embodiment, the fragment is YMIMVKCWMI (SEQ ID No: 57). In another embodiment, the fragment is ELVSEFSRM (SEQ ID No: 58). In another embodiment, the fragment is ELVSEFSRMARDPQ (SEQ ID No: 59). In another embodiment, the fragment is YLVPQQGFFC (SEQ ID No: 60).

In another embodiment, the present invention provides a method of eliciting an anti-Her-2 immune response in a subject, comprising administering to the subject a recombinant peptide of the present invention, thereby eliciting an anti-Her-2 immune response in a subject.

In another embodiment, the present invention provides a method of eliciting an anti-Her-2 immune response in a subject, comprising administering to the subject a nucleotide molecule of the present invention, thereby eliciting an anti-Her-2 immune response in a subject.

In another embodiment, the present invention provides a method of treating a Her-2-expressing tumor in a subject, comprising administering to the subject a recombinant peptide of the present invention, thereby treating a Her-2-expressing tumor in a subject.

In another embodiment, the present invention provides a method of treating a Her-2-expressing tumor in a subject, comprising administering to the subject a nucleotide molecule of the present invention, thereby treating a Her-2-expressing tumor in a subject.

In another embodiment, the present invention provides a method of reducing an incidence of a Her-2-expressing tumor in a subject, comprising administering to the subject a recombinant peptide of the present invention, thereby reducing an incidence of a Her-2-expressing tumor in a subject.

In another embodiment, the present invention provides a method of reducing an incidence of a Her-2-expressing tumor in a subject, comprising administering to the subject a nucleotide molecule of the present invention, thereby reducing an incidence of a Her-2-expressing tumor in a subject.

### BRIEF DESCRIPTION OF THE FIGURES

FIG. 1. Schematic representation of pGG55, used to construct the Lm-Δ-LLO-HER-2 vaccines.

FIG. 2. Recombinant Listeria monocytogenes is capable of secreting each of Her-2 fragments as a ΔLLO-fusion protein. (A) Map of rat Her-2 fragments. (B) Confirmation of secretion of the fusion peptides by Western blot. Marker (lane 1), Lm-ΔLLO-E7 (lane 2), Lm-ΔLLO-EC1 (lane 3), Lm-ΔLLO-EC2 (lane 4), Lm-ΔLLO-EC3 (lane 5), Lm-ΔLLO-IC1 (lane 6), and Lm-ΔLLO-IC2 (lane 7).

FIG. 3. Lm-LLO-HER-2/neu vaccines delay onset of spontaneous tumor growth in FVB/N HER-2/neu transgenic mice. A. PBS and Lm-LLO-NYESO-1 groups; B. Lm-LLO-EC1, Lm-LLO-EC2, and Lm-LLO-EC3 groups; C. Lm-LLO-IC1 and Lm-LLO-IC2 groups.

FIG. 4. Part 1: Mutations in the HER-2/neu extracellular domain mapped onto the human Her-2/neu extracellular structure. EC1, EC2, and EC3 domains were orange, green, and cyan in original. Conserved mutations (blue in original), non-conserved mutations (red in original), and deletions (magenta in original) are depicted. Also see Table 1. Part 2: Grayscale version of Part 1. Mutations are depicted in black; remainder of molecule is gray. EC1 corresponds to top encircled region. EC2 corresponds to middle encircled region, excluding 3 beta-strands indicated by arrows. EC3 corresponds to 3 beta-strands indicated by arrows and bottom encircled region.

FIG. 5. Part 1: Mutations mapped onto the structure of a model of the rat HER-2/neu protein tyrosine kinase domain. The ATP-binding and catalytic domains are identified. * denotes the binding positions of the ATPs. Mutations identified are as follows: 1: Y740A; 2: T764D; 3: Y786A; 4: L801 deleted; 5: L874R; and 6: V978L. Part 2: Grayscale version of Part 1. Mutations are depicted in black; remainder of molecule is gray.

FIG. 6. Residues mutated in the kinase domain correspond to a residue within a CD8⁺ T cell epitope. Diamonds: PBS-vaccinated mice; squares: splenocytes from Lm-LLO-IC1 vaccinated mice. 3T3-wt target cells were pulsed with the following peptides: A. GSGAFGTVYK (732-741); B. AFGTVYKGI (735-743); C. PYVSRLLGI (785-793); D. TSPKANKEI (764-772); E. RPRFRELVSE (971-980); F. STVQLVTQL (797-805); G. KITDFGLARL (865-874). Mutated residues are underlined.

FIG. 7. Schematic representation of human Her-2 fragments used to create LLO-human Her-2 vaccines.

FIG. 8. Comparison of sequential, mixed and individual administration of Her-2 fragments for delaying onset of spontaneous tumor growth in FVB/N HER-2/neu transgenic mice.

FIG. 9. A. Western blot demonstrating that Lm-ActA-E7 secretes E7. Lane 1: Lm-LLO-E7; lane 2: Lm-ActA-E7.001; lane 3; Lm-ActA-E7-2.5.3; lane 4: Lm-ActA-E7-2.5.4. B. Tumor size in mice administered Lm-ActA-E7 (rectangles), Lm-E7 (ovals), Lm-LLO-E7 (X), and naive mice (non-vaccinated; solid triangles).

FIG. 10. A. schematic representation of the plasmid inserts used to create 4 LM vaccines. Lm-LLO-E7 insert contains all of the Listeria genes used. It contains the hly promoter, the first 1.3 kb of the hly gene (which encodes the protein LLO), and the HPV-16 E7 gene. The first 1.3 kb of hly includes the signal sequence (ss) and the PEST region. Lm-PEST-E7 includes the hly promoter, the signal sequence, and PEST and E7 sequences but excludes the remainder of the truncated LLO gene. Lm-ΔPEST-E7 excludes the PEST region, but contains the hly promoter, the signal sequence, E7, and the remainder of the truncated LLO. Lm-E7epi has only the hly promoter, the signal sequence, and E7. B. Top panel: Listeria constructs containing PEST regions induce tumor regression. Bottom panel: Average tumor sizes at day 28 post-tumor challenge in 2 separate experiments. C. Listeria constructs containing PEST regions induce a higher percentage of E7-specific lymphocytes in the spleen. Average and SE of data from 3 experiments are depicted.

Table 1. Specific mutations occurring in spontaneous tumors after vaccination of FVB/N HER-2/neu transgenic mice. 2 tumor samples were sequenced from each vaccine group. Underlined residues fall within the HER-2/neu protein tyrosine kinase domain.

Table 2. There is a 10-fold increase in the probability of a mutation occurring in a vaccine-targeted region.

Table 3. Human Her-2 epitopes.

Table 4. Tumor sizes and health status of the mice from Example 7. UL=Upper left, UR=Upper right, UL/UR=Mammary gland Zone I, mid-L/mid-R=Mammary gland Zone II/III, LL/LR=Mammary gland Zone IV/V.

### DETAILED DESCRIPTION OF THE INVENTION

The present invention provides methods of inducing an immune response in a subject against a protein of interest and treating and suppressing a formation of a tumor comprising a tumor-associated protein, comprising sequential administration of fragments of the protein. The present invention also provides Her-2 protein fragments, recombinant peptides comprising same, nucleotide molecules encoding same, recombinant vaccine vectors and immunogenic compositions comprising same, and methods for inducing immune response and treating cancer, comprising same.

In one embodiment, the present invention provides a method of inducing an immune response in a subject against a protein of interest, the method comprising the steps of: (A) administering to the subject a vaccine comprising a first recombinant peptide, the first recombinant peptide comprising a first fragment of the protein of interest; and (B) after the conclusion of step (A), administering to the subject a vaccine comprising a second recombinant peptide, the second recombinant peptide comprising a second fragment of the protein of interest, thereby inducing an immune response in a subject against a protein of interest. In another embodiment, the protein of interest is a tumor-associated protein. In another embodiment, the protein of interest is an endogenous growth factor. In another embodiment, the protein of interest is a vaso-active peptide or protein. In another embodiment, the protein of interest is any other protein known in the art. Each possibility represents a separate embodiment of the present invention.

“Recombinant” refers, in another embodiment, to a peptide produce by recombinant technology. In another embodiment, the term refers to a synthetic peptide. In another embodiment, the term refers to a non-naturally occurring peptide. Each possibility represents a separate embodiment of the present invention.

The terms “peptide” and “recombinant peptide” refer, in another embodiment, to a peptide or polypeptide of any length. In another embodiment, the length of the peptide or recombinant peptide of the present invention is at least 8 amino acids (AA). In another embodiment, the length is more than 8 AA. In another embodiment, the length is at least 9 AA. In another embodiment, the length is more than 9 AA. In another embodiment, the length is at least 10 AA. In another embodiment, the length is more than 10 AA. In another embodiment, the length is at least 11 AA. In another embodiment, the length is more than 11 AA. In another embodiment, the length is at least 12 AA. In another embodiment, the length is more than 12 AA. In another embodiment, the length is at least about 14 AA. In another embodiment, the length is more than 14 AA. In another embodiment, the length is at least about 16 AA. In another embodiment, the length is more than 16 AA. In another embodiment, the length is at least about 18 AA. In another embodiment, the length is more than 18 AA. In another embodiment, the length is at least about 20 AA. In another embodiment, the length is more than 20 AA. In another embodiment, the length is at least about 25 AA. In another embodiment, the length is more than 25 AA. In another embodiment, the length is at least about 30 AA. In another embodiment, the length is more than 30 AA. In another embodiment, the length is at least about 40 AA. In another embodiment, the length is more than 40 AA. In another embodiment, the length is at least about 50 AA. In another embodiment, the length is more than 50 AA. In another embodiment, the length is at least about 70 AA. In another embodiment, the length is more than 70 AA. In another embodiment, the length is at least about 100 AA. In another embodiment, the length is more than 100 AA. In another embodiment, the length is at least about 150 AA. In another embodiment, the length is more than 150 AA. In another embodiment, the length is at least about 200 AA. In another embodiment, the length is more than 200 AA.

In another embodiment, the length is about 8-50 AA. In another embodiment, the length is about 8-70 AA. In another embodiment, the length is about 8-100 AA. In another embodiment, the length is about 8-150 AA. In another embodiment, the length is about 8-200 AA. In another embodiment, the length is about 8-250 AA. In another embodiment, the length is about 8-300 AA. In another embodiment, the length is about 8-400 AA. In another embodiment, the length is about 8-500 AA. In another embodiment, the length is about 9-50 AA. In another embodiment, the length is about 9-70 AA. In another embodiment, the length is about 9-100 AA. In another embodiment, the length is about 9-150 AA. In another embodiment, the length is about 9-200 AA. In another embodiment, the length is about 9-250 AA. In another embodiment, the length is about 9-300 AA. In another embodiment, the length is about 10-50 AA. In another embodiment, the length is about 10-70 AA. In another embodiment, the length is about 10-100 AA. In another embodiment, the length is about 10-150 AA. In another embodiment, the length is about 10-200 AA. In another embodiment, the length is about 10-250 AA. In another embodiment, the length is about 10-300 AA. In another embodiment, the length is about 10-400 AA. In another embodiment, the length is about 10-500 AA. In another embodiment, the length is about 11-50 AA. In another embodiment, the length is about 11-70 AA. In another embodiment, the length is about 11-100 AA. In another embodiment, the length is about 11-150 AA. In another embodiment, the length is about 11-200 AA. In another embodiment, the length is about 11-250 AA. In another embodiment, the length is about 11-300 AA. In another embodiment, the length is about 11-400 AA. In another embodiment, the length is about 11-500 AA. In another embodiment, the length is about 12-50 AA. In another embodiment, the length is about 12-70 AA. In another embodiment, the length is about 12-100 AA. In another embodiment, the length is about 12-150 AA. In another embodiment, the length is about 12-200 AA. In another embodiment, the length is about 12-250 AA. In another embodiment, the length is about 12-300 AA. In another embodiment, the length is about 12-400 AA. In another embodiment, the length is about 12-500 AA. In another embodiment, the length is about 15-50 AA. In another embodiment, the length is about 15-70 AA. In another embodiment, the length is about 15-100 AA. In another embodiment, the length is about 15-150 AA. In another embodiment, the length is about 15-200 AA. In another embodiment, the length is about 15-250 AA. In another embodiment, the length is about 15-300 AA. In another embodiment, the length is about 15-400 AA. In another embodiment, the length is about 15-500 AA. In another embodiment, the length is about 8-400 AA. In another embodiment, the length is about 8-500 AA. In another embodiment, the length is about 20-50 AA. In another embodiment, the length is about 20-70 AA. In another embodiment, the length is about 20-100 AA. In another embodiment, the length is about 20-150 AA. In another embodiment, the length is about 20-200 AA. In another embodiment, the length is about 20-250 AA. In another embodiment, the length is about 20-300 AA. In another embodiment, the length is about 20-400 AA. In another embodiment, the length is about 20-500 AA. In another embodiment, the length is about 30-50 AA. In another embodiment, the length is about 30-70 AA. In another embodiment, the length is about 30-100 AA. In another embodiment, the length is about 30-150 AA. In another embodiment, the length is about 30-200 AA. In another embodiment, the length is about 30-250 AA. In another embodiment, the length is about 30-300 AA. In another embodiment, the length is about 30-400 AA. In another embodiment, the length is about 30-500 AA. In another embodiment, the length is about 40-50 AA. In another embodiment, the length is about 40-70 AA. In another embodiment, the length is about 40-100 AA. In another embodiment, the length is about 40-150 AA. In another embodiment, the length is about 40-200 AA. In another embodiment, the length is about 40-250 AA. In another embodiment, the length is about 40-300 AA. In another embodiment, the length is about 40-400 AA. In another embodiment, the length is about 40-500 AA. In another embodiment, the length is about 50-70 AA. In another embodiment, the length is about 50-100 AA. In another embodiment, the length is about 50-150 AA. In another embodiment, the length is about 50-200 AA. In another embodiment, the length is about 50-250 AA. In another embodiment, the length is about 50-300 AA. In another embodiment, the length is about 50-400 AA. In another embodiment, the length is about 50-500 AA. In another embodiment, the length is about 70-100 AA. In another embodiment, the length is about 70-150 AA. In another embodiment, the length is about 70-200 AA. In another embodiment, the length is about 70-250 AA. In another embodiment, the length is about 70-300 AA. In another embodiment, the length is about 70-400 AA. In another embodiment, the length is about 70-500 AA. In another embodiment, the length is about 100-150 AA. In another embodiment, the length is about 100-200 AA. In another embodiment, the length is about 100-250 AA. In another embodiment, the length is about 100-300 AA. In another embodiment, the length is about 100-400 AA. In another embodiment, the length is about 100-500 AA. Each possibility represents a separate embodiment of the present invention.

The protein of interest of methods and compositions of the present invention is, in another embodiment, an antigenic protein. In another embodiment, the antigenic peptide is a fragment of an antigenic protein. In another embodiment, the antigenic peptide is an immunogenic peptide derived from tumor. In another embodiment, the antigenic peptide is an immunogenic peptide derived from metastasis. In another embodiment, the antigenic peptide is an immunogenic peptide derived from cancerous cells. In another embodiment, the antigenic peptide is a pro-angiogenesis immunogenic peptide. In another embodiment, the

In another embodiment, the protein of interest of methods and compositions of the present invention is a tumor-associated protein. “Tumor-associated protein” refers, in another embodiment, to a protein that is expressed by a tumor. In another embodiment, the protein is an oncoprotein. In another embodiment, the protein is not expressed in healthy cells. In another embodiment, the protein is not expressed in healthy cells, with the exception of embryonic cells. In another embodiment, the protein is not expressed in healthy cells, with the exception of genomic cells. In another embodiment the tumor-associated protein is expressed in healthy cells from disposable tissues. In another embodiment, expression of the protein is upregulated in tumor cells relative to healthy cells. In another embodiment, activity of the protein is upregulated in tumor cells relative to healthy cells. In another embodiment, the protein is mutated in tumor cells. In another embodiment, a tumor-associated protein mediates an activity required for oncogenic transformation. In another embodiment a tumor-associated protein enhances tumor growth through cell signaling. In another embodiment a tumor-associated protein is required to maintain telomere integrity. In another embodiment, a tumor-associated protein mediates an activity required for maintenance of a transformed phenotype. Each possibility represents a separate embodiment of the present invention.

In another embodiment, the protein of interest is Human Papilloma Virus-E7 (HPV-E7) antigen; e.g. from an HPV 16 or HPV18. In another embodiment, the protein of interest is HPV-E6. In another embodiment, the protein of interest is a Her/2-neu antigen; e.g. GenBank Accession Nos. M16789.1, M16790.1, M16791.1, and M16792.1. In another embodiment, the protein of interest is Prostate Specific Antigen (PSA). In another embodiment, the protein of interest is Stratum Corneum Chymotryptic Enzyme (SCCE) antigen; e.g. GenBank Accession No. NM_{—}005046 and NM_{—}139277. In another embodiment, the protein of interest is Wilms tumor antigen 1. In another embodiment, the protein of interest is hTERT or Telomerase (GenBank Accession. Nos. NM003219 (variant 1), NM198255 (variant 2), NM 198253 (variant 3), and NM 198254 (variant 4). In another embodiment, the protein of interest is Proteinase 3 (e.g. GenBank Accession Nos. M29142, M75154, M96839, X55668, NM 00277, M96628 and X56606). In another embodiment, the protein of interest is Tyrosinase Related Protein 2 (TRP2). In another embodiment, the protein of interest is High Molecular Weight Melanoma Associated Antigen (HMW-MAA). In another embodiment, the protein of interest is Testisin. In another embodiment, the protein of interest is NY-ESO-1 antigen. In another embodiment, the protein of interest is any other protein of interest known in the art. Each possibility represents a separate embodiment of the present invention.

In other embodiments, the antigen is one of the following tumor antigens: any of the various MAGEs (Melanoma-Associated Antigen E), including MAGE 1 (e.g., GenBank Accession No. M77481), MAGE 2 (e.g., GenBank Accession No. U03735), MAGE 3, MAGE 4, gp-100, tyrosinase, MART-1, HSP-70, and beta-HCG; a tyrosinase; mutant ras; mutant p53 (e.g., GenBank Accession No. X54156 and AA494311); and p97 melanoma antigen (e.g., GenBank Accession No. M12154). Other tumor-specific antigens include the Ras peptide and p53 peptide associated with advanced cancers, MUC1 antigen associated with breast carcinoma (e.g., GenBank Accession No. J0365 1), CEA (carcinoembryonic antigen) associated with colorectal cancer (e.g., GenBank Accession No. X983 11), gp100 (e.g., GenBank Accession No. S73003) or MART1 antigens associated with melanoma, and the prostate-specific antigen (KLK3) associated with prostate cancer (e.g., GenBank Accession No. X14810). The p53 gene sequence is known (See e.g., Harris et al. (1986) Mol. Cell. Biol., 6:4650-4656) and is deposited with GenBank under Accession No. M14694. Tumor antigens encompassed by the present invention further include, but are not limited to NY-ESO-1 (e.g. GenBank Accession No. U87459), WT-1 (e.g. GenBank Accession Nos. NM000378 (variant A), NM024424 (variant B), NM 024425 (variant C), and NM024426 (variant D)), Her-2/Neu (e.g. GenBank Accession Nos. M16789.1, M16790.1, M16791.1, M16792.1). Thus, the present invention can be used as immunotherapeutics for cancers including, but not limited to, cervical, breast, colorectal, prostate, lung cancers, and for melanomas.

In another embodiment, the protein of interest is an infectious disease antigen.

In other embodiments, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, HIV env protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, human papilloma virus antigens E1 and E2 from type HPV-16, -18, -31, -33, -35 or -45 human papilloma viruses, the tumor antigens CEA, the ras protein, mutated or otherwise, the p53 protein, mutated or otherwise

In another embodiment, the antigen of methods and compositions of the present invention includes but is not limited to antigens from the following infectious diseases, measles, mumps, rubella, poliomyelitis, hepatitis A, B (e.g., GenBank Accession No. E02707), and C (e.g., GenBank Accession No. E06890), as well as other hepatitis viruses, type A influenza, other types of influenza, adenovirus (e.g., types 4 and 7), rabies (e.g., GenBank Accession No. M34678), yellow fever, Japanese encephalitis (e.g., GenBank Accession No. E07883), dengue (e.g., GenBank Accession No. M24444), hantavirus, and HIV (e.g., GenBank Accession No. U18552). Bacterial and parasitic antigens will be derived from known causative agents responsible for diseases including, but not limited to, diphtheria, pertussis (e.g., GenBank Accession No. M35274), tetanus (e.g., GenBank Accession No. M64353), tuberculosis, bacterial and fungal pneumonias (e.g., Haemophilus influenzae, Pneumocystis carinii, etc.), cholera, typhoid, plague, shigellosis, salmonellosis (e.g., GenBank Accession No. L03833), Legionnaire's Disease, Lyme disease (e.g., GenBank Accession No. U59487), malaria (e.g., GenBank Accession No. X53832), hookworm, onchocerciasis (e.g., GenBank Accession No. M27807), schistosomiasis (e.g., GenBank Accession No. L08198), trypanosomiasis, leshmaniasis, giardiasis (e.g., GenBank Accession No. M33641), amoebiasis, filariasis (e.g., GenBank Accession No. J03266), borreliosis, and trichinosis.

In another embodiment, the protein of interest is an endogenous growth factor. In another embodiment, the protein of interest is a human growth hormone (hGH). In another embodiment, the protein of interest is an insulin-like growth factor. In another embodiment, the protein of interest is an epidermal growth factor. In another embodiment, the protein of interest is an acidic or basic fibroblast growth factor. In another embodiment, the protein of interest is a platelet-derived growth factor. In another embodiment, the protein of interest is a granulocyte-CSF. In another embodiment, the protein of interest is a granulocyte-macrophage colony stimulating factor (GM-CSF). In another embodiment, the protein of interest is a macrophage-CSF. In another embodiment, the protein of interest is ErbB1 (epidermal growth factor receptor or EGFR). In another embodiment, the protein of interest is ErbB3. In another embodiment, the protein of interest is ErbB4. In another embodiment, the protein of interest is any other growth factor known in the art. Each possibility represents a separate embodiment of the present invention.

In another embodiment, the protein of interest is a vaso-active protein or peptide. vasoactive intestinal peptide receptor 1. In another embodiment, the protein of interest is an angiotensin. In another embodiment, the protein of interest is a renin. In another embodiment, the protein of interest is Angiotensin II. In another embodiment, the protein of interest is a Converting Enzyme. In another embodiment, the protein of interest is an Angiotensin receptor. In another embodiment, the protein of interest is a Kinin. In another embodiment, the protein of interest is a Kallikrein. In another embodiment, the protein of interest is a Kininogen. In another embodiment, the protein of interest is a Bradykinin. In another embodiment, the protein of interest is a Lysylbradykinin. In another embodiment, the protein of interest is a Methionyllysylbradykinin. In another embodiment, the protein of interest is an atrial natriuretic peptide (ANP). In another embodiment, the protein of interest is any other vaso-active protein or peptide known in the art. Each possibility represents a separate embodiment of the present invention.

In another embodiment, the protein of interest is any other protein known in the art. Each possibility represents a separate embodiment of the present invention.

In another embodiment, the step of “administering” the first recombinant peptide, second recombinant peptide, etc, consists of a single vaccination with an immunogenic composition comprising the peptide. In another embodiment, 2 vaccinations are administered. In another embodiment, 3 vaccinations are administered. In another embodiment, 4 vaccinations are administered. In another embodiment, more than 4 vaccinations are administered. In another embodiment, the first recombinant peptide, second recombinant peptide, etc is administered until a clinical endpoint is reached. In another embodiment, the clinical endpoint is tumor regression. In another embodiment, the clinical endpoint is remission. In another embodiment, the clinical endpoint is any other clinical endpoint known in the art. Each possibility represents a separate embodiment of the present invention.

In another embodiment of methods and compositions of the present invention, the tumor-associated protein is a kinase. In another embodiment, the tumor-associated protein is a protein kinase. In another embodiment, the tumor-associated protein is a tyrosine kinase. In another embodiment, the tyrosine kinase is an insulin receptor. In another embodiment, the tyrosine kinase is an EGF receptor. In another embodiment, the tyrosine kinase is an FGF receptor. In another embodiment, the tumor-associated protein is a serine/threonine kinase. In another embodiment, the tumor-associated protein is a phosphatase. In another embodiment, the tumor-associated protein is a tyrosine phosphatase. In another embodiment, the tumor-associated protein is a telomerase. In another embodiment, the tumor-associated protein is TERT protein. In another embodiment, the tumor-associated protein is a transcriptional activator protein. In another embodiment, the tumor-associated protein interacts with an adapter protein. In another embodiment, the tumor-associated protein is a cell cycle activator protein. In another embodiment, the tumor-associated protein is a MAP kinase. In another embodiment, the tumor-associated protein is a MAP kinase-activating protein. In another embodiment, the tumor-associated protein is a phospholipase. In another embodiment, the tumor-associated protein is a lipid kinase. In another embodiment, the tumor-associated protein is a G-protein regulator protein. In another embodiment, the tumor-associated protein is a GTPase activating protein. In another embodiment, the tumor-associated protein is a steroid/thyroid hormone receptor. In another embodiment, the tumor-associated protein mediates oncogenesis by downregulating a tumor suppressor gene. In another embodiment, the tumor-associated protein is a viral oncogene. In another embodiment, the tumor-associated protein is any other type of protein known in the art that mediates an oncogenic function.

The first fragment of the tumor-associated protein that is administered is, in another embodiment, homologous to a cellular protein expressed by a non-tumor cell (“non-tumor protein”) of the subject, or a domain of the cellular protein. In another embodiment, the first fragment is homologous to a family of proteins expressed by a non-tumor cell or a domain thereof. In another embodiment, the second fragment is homologous to a non-tumor protein or a domain thereof. In another embodiment, the second fragment is homologous to a family of proteins expressed by a non-tumor cell or a domain thereof. In another embodiment, if a third fragment is administered, the third fragment is homologous to a non-tumor protein or a domain thereof. In another embodiment, the third fragment is homologous to a family of proteins expressed by a non-tumor cell or a domain thereof. In another embodiment, more than 1 of the administered fragments is homologous to a non-tumor protein or a domain thereof. In another embodiment, 2 of the administered fragments are homologous to a non-tumor protein or a domain thereof. In another embodiment, more than 2 of the administered fragments are homologous to a non-tumor protein or a domain thereof. In another embodiment, 3 of the administered fragments are homologous to a non-tumor protein or a domain thereof. In another embodiment, a single non-tumor protein or family is homologous to more than 1 of the administered fragments. In another embodiment, more than 1 non-tumor protein or family is homologous to the more than 1 administered fragments. Each possibility represents a separate embodiment of the present invention.

In another embodiment, the cellular protein that is homologous to the tumor-associated protein is expressed in a quantity detectable by an assay used in the art. In another embodiment, the cellular protein is expressed in a quantity sufficient to induce immune tolerance in the subject. In another embodiment, the cellular protein is expressed in a quantity sufficient to induce immune tolerance in a majority of subjects. In another embodiment, the cellular protein is expressed in a quantity sufficient to induce immune tolerance in a significant fraction of subjects.

The assay used to detect expression of the cellular protein is, in another embodiment, Western blotting. In another embodiment the assay is immunoprecipitation. In another embodiment, the assay is Northern blotting. In another embodiment the assay is RT-PCR. In another embodiment, the assay is fluorescence-activated cell sorting (FACS). In another embodiment, the assay is Enzyme-Linked Immunosorbent Assay (ELISA). In another embodiment, the assay is any other assay known in the art. Each possibility represents a separate embodiment of the present invention.

Methods of measuring immune responses and immune tolerance are well known in the art, and include, for example, flow cytometry, target cell lysis assays (in another embodiment, chromium release assay) the use of tetramers, and others. These methods are described, for example, in Current Protocols in Immunology (John E. Coligan et al, Copyright ©2006 by John Wiley & Sons, Inc). Each method represents a separate embodiment of the present invention.

“Immune tolerance” refers, in another embodiment, to the lack of a detectable immune response by a detection method used in the art. The detection method is, in other embodiments, any of the methods enumerated herein. In another embodiment, “immune tolerance” refers to a state wherein a cell expressing the protein of interest is not rejected by the host immune system. In another embodiment, prior to vaccination, expression of the protein of interest by a tumor cell does not result in elimination of the tumor cell. In another embodiment, prior to vaccination, a tumor expressing the protein of interest is not infiltrated by tumor-infiltrating T lymphocytes. Each possibility represents a separate embodiment of the present invention.

In another embodiment of methods and compositions of the present invention, the first fragment that is administered comprises a domain that mediates an oncogenic function of the tumor-associated protein. In another embodiment, the first fragment is composed primarily of the functional domain. In another embodiment, the second fragment comprises a domain that mediates an oncogenic function. In another embodiment, the second fragment is composed primarily of the “functional domain” (that is, the domain with oncogenic function). In another embodiment, if a third fragment is administered, the third fragment comprises a domain that mediates an oncogenic function of the tumor-associated protein. In another embodiment, the third fragment is composed primarily of the functional domain. In another embodiment, 2 of the administered fragments comprise domains that mediate an oncogenic function. In another embodiment, more than 1 of the administered fragments comprise a domain that mediates an oncogenic function. In another embodiment, more than 2 of the administered fragments comprise a domain that mediates an oncogenic function. In another embodiment, 2 of the administered fragments comprise a domain that mediates an oncogenic function. In another embodiment, more than 3 of the administered fragments comprise a domain that mediates an oncogenic function. In another embodiment, all the administered fragments comprise a domain that mediates an oncogenic function. In another embodiment, more than 1 functional domain is included in different fragments that are administered. In another embodiment, 2 functional domains are included in different fragments that are administered. In another embodiment, more than 2 functional domains are included in different fragments that are administered. In another embodiment, 3 functional domains are included in different fragments that are administered. In another embodiment, more than 3 functional domains are included in different fragments that are administered. Each possibility represents a separate embodiment of the present invention.

“Composed primarily” refers, in another embodiment, to more than 50% of the fragment being the functional domain. In another embodiment, 60% or more of the fragment is the functional domain. In another embodiment, more than 60% of the fragment is the functional domain. In another embodiment, 70% or more of the fragment is the functional domain. In another embodiment, more than 70% of the fragment is the functional domain. In another embodiment, 80% or more of the fragment is the functional domain. In another embodiment, more than 80% of the fragment is the functional domain. In another embodiment, 90% or more of the fragment is the functional domain. In another embodiment, more than 90% of the fragment is the functional domain. Each possibility represents a separate embodiment of the present invention.

In another embodiment of methods and compositions of the present invention, the first fragment that is administered overlaps with a domain that mediates an oncogenic function of the tumor-associated protein. In another embodiment, the second fragment overlaps with a domain that mediates an oncogenic function. In another embodiment, if a third fragment is administered, the third fragment overlaps with a domain that mediates an oncogenic function of the tumor-associated protein. In another embodiment, 2 of the administered fragments overlap with domains that mediate an oncogenic function. In another embodiment, more than 1 of the administered fragments overlap with a domain that mediates an oncogenic function. In another embodiment, more than 2 of the administered fragments overlap with a domain that mediates an oncogenic function. In another embodiment, 3 of the administered fragments overlap with a domain that mediates an oncogenic function. In another embodiment, more than 3 of the administered fragments overlap with a domain that mediates an oncogenic function. In another embodiment, all the administered fragments overlap with a domain that mediates an oncogenic function. In another embodiment, more than 1 functional domain overlaps different fragments that are administered. In another embodiment, 2 functional domains overlap with different fragments that are administered. In another embodiment, more than 2 functional domains overlap with different fragments that are administered. In another embodiment, 3 functional domains overlap with different fragments that are administered. In another embodiment, more than 3 functional domains overlap with different fragments that are administered. Each possibility represents a separate embodiment of the present invention.

“Overlaps” refers, in another embodiment, to 10% of the functional domain being contained within the fragment that is administered. In another embodiment, 20% of the functional domain is contained within the fragment. In another embodiment, 30% of the functional domain is contained within the fragment. In another embodiment, 40% of the functional domain is contained within the fragment. In another embodiment, 50% of the functional domain is contained within the fragment. In another embodiment, 60% of the functional domain is contained within the fragment. In another embodiment, 70% of the functional domain is contained within the fragment. In another embodiment, 80% of the functional domain is contained within the fragment. In another embodiment, 90% of the functional domain is contained within the fragment. In another embodiment, more than 90% of the functional domain is contained within the fragment. Each possibility represents a separate embodiment of the present invention.

The domain that mediates an oncogenic function is, in another embodiment, a kinase domain. In another embodiment, the domain is a tyrosine kinase domain. In another embodiment, the tyrosine kinase is PDGF. In another embodiment, the tyrosine kinase is an insulin receptor. In another embodiment, the tyrosine kinase is an EGF receptor. In another embodiment, the tyrosine kinase is an FGF receptor. In another embodiment, the domain is a serine/threonine kinase domain. In another embodiment, the serine/threonine kinase is activin. In another embodiment, the domain is a serine/threonine kinase a TGF-β receptor. In another embodiment, the domain is a phosphatase domain. In another embodiment, the domain is a tyrosine phosphatase domain. In another embodiment, the tyrosine phosphatase is CD45 (cluster determinant-45) protein. In another embodiment, the domain is a telomerase domain. In another embodiment, the domain is coupled to a GTP-binding and hydrolyzing protein (G-protein). In another embodiment, the domain is a transcriptional activator domain. In another embodiment, the domain is a protein-protein interaction domain. In another embodiment, the domain is an SH2 domain. In another embodiment, the domain is an SH3 domain. In another embodiment, the domain is a docking domain for an adapter protein. In another embodiment, the adaptor protein is growth factor receptor-binding protein 2 (Grb2). In another embodiment, the domain is a cell cycle activation domain. In another embodiment, the domain is a MAP kinase domain. In another embodiment, the domain is a MAP kinase-activating domain. In another embodiment, the domain is a phospholipase domain. In another embodiment, the phospholipase is phospholipase C-γ. In another embodiment, the phospholipase is PLD. In another embodiment, the phospholipase is PLA₂. In another embodiment, the domain is a lipid kinase domain. In another embodiment, the lipid kinase is PI-3K. In another embodiment, the domain is a G-protein regulator domain. In another embodiment, the domain is a GTPase activating domain. In another embodiment, the GTPase activating protein (GAP) is Ras. In another embodiment, the domain is an oncogenic domain of a steroid/thyroid hormone receptor protein. In another embodiment, the domain is a ligand binding domain thereof. In another embodiment, the domain is a transcriptional activator domain thereof. In another embodiment, the domain is any other type of domain known in the art that mediates an oncogenic function.

In another embodiment, the oncogenic function referred to herein is promotion of growth. In another embodiment, the oncogenic function is promotion of rapid growth. In another embodiment, the oncogenic function is promotion of constitutive growth. In another embodiment, the oncogenic function is promotion of uncontrolled growth. In another embodiment, the oncogenic function is cell cycle activation. In another embodiment, the oncogenic function is telomerase lengthening. In another embodiment, the oncogenic function is an activity required for metastases. In another embodiment, the oncogenic function is invasiveness. In another embodiment, the oncogenic function is detachment from neighboring cells. In another embodiment, the oncogenic function is loss of anchorage dependence. In another embodiment, the oncogenic function is proteolysis of the extracellular matrix. In another embodiment, the oncogenic function is proteolysis of the basement membrane. In another embodiment, the oncogenic function is induction of angiogenesis. In another embodiment the oncogenic function is degradation of host tumor suppressor genes. In another embodiment, the oncogenic function is any other oncogenic function known in the art. Each possibility represents a separate embodiment of the present invention.

In another embodiment, a method of the present invention further comprises the step of administering to the subject a vaccine comprising a third recombinant peptide, the third recombinant peptide comprising a third fragment of the tumor-associated protein. In another embodiment, a fourth recombinant peptide, comprising a fourth fragment of the tumor-associated protein, is administered. In another embodiment, a fifth recombinant peptide, comprising a fifth fragment of the tumor-associated protein, is administered. In another embodiment, more than 5 recombinant peptides, comprising more than 5 fragments of the tumor-associated protein, are administered. In another embodiment, administration of each fragment follows the completion of vaccination with the previous fragments. Each possibility represents a separate embodiment of the present invention.

In another embodiment, the first recombinant peptide is expressed by a live recombinant vaccine vector in the immunogenic composition that is administered. In another embodiment, the second recombinant peptide is expressed by a live recombinant vaccine vector. In another embodiment, wherein a third recombinant peptide is administered, the third recombinant peptide is expressed by a live recombinant vaccine vector. In another embodiment, more than 1 of the recombinant peptides are expressed by a live recombinant vaccine vector. In another embodiment, 2 of the recombinant peptides are expressed by a live recombinant vaccine vector. In another embodiment, more than 2 of the recombinant peptides are expressed by a live recombinant vaccine vector. In another embodiment, 3 of the recombinant peptides are expressed by a live recombinant vaccine vector. In another embodiment, more than 3 of the recombinant peptides are expressed by a live recombinant vaccine vector. In another embodiment, all the recombinant peptides are expressed by a live recombinant vaccine vector. Each possibility represents a separate embodiment of the present invention.

In another embodiment, the first recombinant peptide is expressed by a recombinant Listeria strain in the immunogenic composition that is administered. In another embodiment, the second recombinant peptide is expressed by a recombinant Listeria strain. In another embodiment, wherein a third recombinant peptide is administered, the third recombinant peptide is expressed by a recombinant Listeria strain. In another embodiment, more than 1 of the recombinant peptides are expressed by a recombinant Listeria strain. In another embodiment, 2 of the recombinant peptides are expressed by a recombinant Listeria strain. In another embodiment, more than 2 of the recombinant peptides are expressed by a recombinant Listeria strain. In another embodiment, 3 of the recombinant peptides are expressed by a recombinant Listeria strain. In another embodiment, more than 3 of the recombinant peptides are expressed by a recombinant Listeria strain. In another embodiment, all the recombinant peptides are expressed by a recombinant Listeria strain. Each possibility represents a separate embodiment of the present invention.

In another embodiment, the first recombinant peptide further comprises a heterologous peptide that enhances immunogenicity of the fragment of the tumor-associated protein. In another embodiment, the tumor-associated protein fragment is fused to heterologous peptide. In another embodiment, the tumor-associated protein fragment is embedded within the heterologous peptide, as exemplified herein (DP-L2851, Example 7). Each possibility represents a separate embodiment of the present invention.

“Heterologous” refers, in another embodiment, to a peptide that is not derived from the target tumor-associated protein. In another embodiment, the term refers to a peptide that is not derived from a protein expressed by the subject. In another embodiment, the heterologous peptide is derived from another species. In another embodiment, the heterologous peptide is a synthetic peptide. In another embodiment, the heterologous peptide is a proteasome-targeting peptide.

In another embodiment, the heterologous peptide is a bacterial protein. In another embodiment, the heterologous peptide is a viral protein. In another embodiment, the heterologous peptide is a protein from another type of infectious disease. In another embodiment, the heterologous peptide is a listeriolysin (LLO) peptide. In another embodiment, the heterologous peptide is an N-terminal fragment of LLO. In another embodiment, the heterologous peptide is an ActA protein. In another embodiment, the heterologous peptide is an ActA fragment. In another embodiment, the heterologous peptide is a PEST-like sequence. In another embodiment, the heterologous peptide is any other type of peptide known in the art. The use of immunogenic peptides to enhance the Immunogenicity of an antigen is well known in the art, and is described, for example, in United States Patent Application Serial No. 2005/0118184, 2006/0110400, and 2005/0226891. Each possibility represents a separate embodiment of the present invention.

In another embodiment, “LLO peptide” and “LLO fragment” refer to an N-terminal fragment of an LLO protein. In another embodiment, the N-terminal fragment is about 400-441 AA in length. In another embodiment, the terms refer to a full-length but non-hemolytic LLO protein. In another embodiment, the terms refer to a non-hemolytic protein containing a point mutation in cysteine 484 of sequence ID No: 34 or a corresponding residue thereof in a homologous LLO protein. Each possibility represents a separate embodiment of the present invention.

In another embodiment, the LLO peptide in the first recombinant peptide is fused to the first fragment of the protein of interest. In another embodiment, the LLO peptide in the second recombinant peptide is fused to the second fragment of the protein of interest. In another embodiment, the LLO peptide in the third recombinant peptide is fused to the third fragment of the protein of interest. In another embodiment, the LLO peptide in the fourth recombinant peptide is fused to the fourth fragment of the protein of interest. In another embodiment, the LLO peptide in each recombinant peptide is fused to the fragment of the protein of interest. Each possibility represents a separate embodiment of the present invention.

In another embodiment, the first fragment of the protein of interest is embedded within the LLO peptide in the first recombinant peptide. In another embodiment, the second fragment of the protein of interest is embedded within the LLO peptide in the second recombinant peptide. In another embodiment, the third fragment of the protein of interest is embedded within the LLO peptide in the third recombinant peptide. In another embodiment, the fourth fragment of the protein of interest is embedded within the LLO peptide in the fourth recombinant peptide. In another embodiment, the fragment of the protein of interest of each recombinant peptide is embedded within the LLO peptide. Each possibility represents a separate embodiment of the present invention.

In another embodiment, a whole LLO protein is utilized in methods and compositions of the present invention. In another embodiment, the whole LLO protein is a non-hemolytic LLO protein.

The LLO protein utilized to construct vaccines of the present invention (in another embodiment, used as the source of the LLO fragment incorporated in the vaccines) has, in another embodiment, the sequence:

MKKIMLVFITLILVSLPIAQQTEAKDASAFNKENSISSMAPPASPPASPKTPIEKKHADEIDKYIQGLD YNKNNVLVYHGDAVTNVPPRKGYKDGNEYIVVEKKKKSINQNNADIQVVNAISSLTYPGALVKA NSELVENQPDVLPVKRDSLTLSIDLPGMTNQDNKIVVKNATKSNVNNAVNTLVERWNEKYAQAY PNVSAKIDYDDEMAYSESQLIAKFGTAFKAVNNSLNVNFGAISEGKMQEEVISFKQIYYNVNVNEP TRPSRFFGKAVTKEQLQALGVNAENPPAYISSVAYGRQVYLKLSTNSHSTKVKAAFDAAVSGKSV SGDVELTNIIKNSSFKAVIYGGSAKDEVQIIDGNLGDLRDILKKGATFNRETPGVPIAYTFNFLKDNE LAVIKNNSEYIETTSKAYTDGKINIDHSGGYVAQFNISWDEVNYDPEGNEIVQHKNWSENNKSKL AHFTSSIYLPGNARNINVYAKECTGLAWEWWRTVIDDRNLPLVKNRNISIWGTTLYPKYSNKVDN PIE (GenBank Accession No. P13128; SEQ ID NO: 34; the nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long.

In another embodiment, a N-terminal fragment of an LLO protein utilized in compositions and methods of the present invention has the sequence:

(SEQ ID NO: 18)

MKKIMLVFITLILVSLPIAQQTEAKDASAFNKENSISSVAPPASPPASP
KTPIEKKHADEIDKYIQGLDYNKNNVLVYHGDAVTNVPPRKGYKDGNEY
IVVEKKKKSINQNNADIQVVNAISSLTYPGALVKANSELVENQPDVLPV
KRDSLTLSIDLPGMTNQDNKIVVKNATKSNVNNAVNTLVERWNEKYAQA
YSNVSAKIDYDDEMAYSESQLIAKFGTAFKAVNNSLNVNFGAISEGKMQ
EEVISFKQIYYNVNVNEPTRPSRFFGKAVTKEQLQALGVNAENPPAYIS
SVAYGRQVYLKLSTNSHSTKVKAAFDAAVSGKSVSGDVELTNIIKNSSF
KAVIYGGSAKDEVQIIDGNLGDLRDILKKGATFNRETPGVPIAYTTNFL
KDNELAVIKNNSEYIETTSKAYTDGKINIDHSGGYVAQFNISWDEVN
YD.

In another embodiment, the LLO fragment has the sequence:

(SEQ ID NO: 19)

MKKIMLVFITLILVSLPIAQQTEAKDASAFNKENSISSVAPPASPPASP
KTPIEKKHADEIDKYIQGLDYNKNNVLVYHGDAVTNVPPRKGYKDGNEY
IVVEKKKKSINQNNADIQVVNAISSLTYPGALVKANSELVENQPDVLPV
KRDSLTLSIDLPGMTNQDNKIVVKNATKSNVNNAVNTLVERWNEKYAQA
YSNVSAKIDYDDEMAYSESQLIAKFGTAFKAVNNSLNVNFGAISEGKMQ
EEVISFKQIYYNVNVNEPTRPSRFFGKAVTKEQLQALGVNAENPPAYIS
SVAYGRQVYLKLSTNSHSTKVKAAFDAAVSGKSVSGDVELTNIIKNSSF
KAVIYGGSAKDEVQIIDGNLGDLRDILKKGATFNRETPGVPIAYTTNFL
KDNELAVIKNNSEYIETTSKAYTDGKINIDHSGGYVAQFNISWDEVN
YD.

In another embodiment, the LLO fragment of methods and compositions of the present invention comprises a PEST-like domain. In another embodiment, an LLO fragment that comprises a PEST sequence is utilized.

In another embodiment, the LLO fragment does not contain the activation domain at the carboxy terminus and does not include cysteine 484. In another embodiment, the LLO fragment is a non-hemolytic fragment. In another embodiment, the LLO fragment is rendered non-hemolytic by deletion or mutation of the activation domain. In another embodiment, the LLO fragment is rendered non-hemolytic by deletion or mutation of cysteine 484. In another embodiment, the LLO fragment is rendered non-hemolytic by deletion or mutation at another location.

In another embodiment, the LLO fragment consists of about the first 441 AA of the LLO protein. In another embodiment, the LLO fragment corresponds to AA 1-441 of an LLO protein disclosed herein. In another embodiment, the LLO fragment consists of about the first 420 AA of LLO. In another embodiment, the LLO fragment corresponds to AA 1-420 of an LLO protein disclosed herein. In another embodiment, the LLO fragment consists of about AA 20-442 of LLO. In another embodiment, the LLO fragment corresponds to AA 20-442 of an LLO protein disclosed herein. In another embodiment, any ΔLLO without the activation domain containing cysteine 484, and in particular without cysteine 484, are suitable for methods and compositions of the present invention.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein.

Each LLO protein and LLO fragment represents a separate embodiment of the present invention.

In another embodiment of methods and compositions of the present invention, an ActA peptide is fused to the tumor-associated protein fragment.

“ActA peptide” refers, in another embodiment, to a full-length ActA protein. In another embodiment, the term refers to an ActA fragment. Each possibility represents a separate embodiment of the present invention.

In another embodiment, the ActA peptide in the first recombinant peptide is fused to the first fragment of the protein of interest. In another embodiment, the ActA peptide in the second recombinant peptide is fused to the second fragment of the protein of interest. In another embodiment, the ActA peptide in the third recombinant peptide is fused to the third fragment of the protein of interest. In another embodiment, the ActA peptide in the fourth recombinant peptide is fused to the fourth fragment of the protein of interest. In another embodiment, the ActA peptide in each recombinant peptide is fused to the fragment of the protein of interest. Each possibility represents a separate embodiment of the present invention.

In another embodiment, the first fragment of the protein of interest is embedded within the ActA peptide in the first recombinant peptide. In another embodiment, the second fragment of the protein of interest is embedded within the ActA peptide in the second recombinant peptide. In another embodiment, the third fragment of the protein of interest is embedded within the ActA peptide in the third recombinant peptide. In another embodiment, the fourth fragment of the protein of interest is embedded within the ActA peptide in the fourth recombinant peptide. In another embodiment, the fragment of the protein of interest of each recombinant peptide is embedded within the ActA peptide. Each possibility represents a separate embodiment of the present invention.

The ActA fragment of methods and compositions of the present invention is, in another embodiment, an N-terminal ActA fragment. In another embodiment, the fragment is any other type of ActA fragment known in the art. Each possibility represents a separate embodiment of the present invention.

In another embodiment, the fragment of an ActA protein has the sequence:

MRAMMVVFITANCITINPDIIFAATDSEDSSLNTDEWEEEKTEEQPSEVNTGPRYETAREVS SRDIKELEKSNKVRNTNKADLIAMLKEKAEKGPNINNNNSEQTENAAINEEASGADRPAIQVERRH PGLPSDSAAEIKKRRKAIASSDSELESLTYPDKPTKVNKKKVAKESVADASESDLDSSMQSADESS PQPLKANQQPFFPKVFKKIKDAGKWVRDKIDENPEVKKAIVDKSAGLIDQLLTKKKSEEVNASDF PPPPTDEELRLALPETPMLLGFNAPATSEPSSFEFPPPPTDEELRLALPETPMLLGFNAPATSEPSSFE FPPPPTEDELEIIRETASSLDSSFTRGDLASLRNAINRHSQNFSDFPPIPTEEELNGRGGRP (SEQ ID NO: 20). In another embodiment, the ActA fragment comprises the sequence set forth in SEQ ID NO: 20. In another embodiment, the ActA fragment is any other ActA fragment known in the art. Each possibility represents a separate embodiment of the present invention.

In another embodiment, the ActA fragment is encoded by a recombinant nucleotide comprising the sequence:

ATGCGTGCGATGATGGTGGTTTTCATTACTGCCAATTGCATTACGATTAACCCCGACATAATA TTTGCAGCGACAGATAGCGAAGATTCTAGTCTAAACACAGATGAATGGGAAGAAGAAAAAA CAGAAGAGCAACCAAGCGAGGTAAATACGGGACCAAGATACGAAACTGCACGTGAAGTAAG TTCACGTGATATTAAAGAACTAGAAAAATCGAATAAAGTGAGAAATACGAACAAAGCAGAC CTAATAGCAATGTTGAAAGAAAAAGCAGAAAAAGGTCCAAATATCAATAATAACAACAGTG AACAAACTGAGAATGCGGCTATAAATGAAGAGGCTTCAGGAGCCGACCGACCAGCTATACAA GTGGAGCGTCGTCATCCAGGATTGCCATCGGATAGCGCAGCGGAAATTAAAAAAAGAAGGA AAGCCATAGCATCATCGGATAGTGAGCTTGAAAGCCTTACTTATCCGGATAAACCAACAAAA GTAAATAAGAAAAAAGTGGCGAAAGAGTCAGTTGCGGATGCTTCTGAAAGTGACTTAGATTC TAGCATGCAGTCAGCAGATGAGTCTTCACCACAACCTTTAAAAGCAAACCAACAACCATTTTT CCCTAAAGTATTTAAAAAAATAAAAGATGCGGGGAAATGGGTACGTGATAAAATCGACGAA AATCCTGAAGTAAAGAAAGCGATTGTTGATAAAAGTGCAGGGTTAATTGACCAATTATTAAC CAAAAAGAAAAGTGAAGAGGTAAATGCTTCGGACTTCCCGCCACCACCTACGGATGAAGAGT TAAGACTTGCTTTGCCAGAGACACCAATGCTTCTTGGTTTTAATGCTCCTGCTACATCAGAAC CGAGCTCATTCGAATTTCCACCACCACCTACGGATGAAGAGTTAAGACTTGCTTTGCCAGAGA CGCCAATGCTTCTTGGTTTTAATGCTCCTGCTACATCGGAACCGAGCTCGTTCGAATTTCCACC GCCTCCAACAGAAGATGAACTAGAAATCATCCGGGAAACAGCATCCTCGCTAGATTCTAGTT TTACAAGAGGGGATTTAGCTAGTTTGAGAAATGCTATTAATCGCCATAGTCAAAATTTCTCTG ATTTCCCACCAATCCCAACAGAAGAAGAGTTGAACGGGAGAGGCGGTAGACCA (SEQ ID NO: 21). In another embodiment, the recombinant nucleotide has the sequence set forth in SEQ ID NO: 21. In another embodiment, the recombinant nucleotide comprises any other sequence that encodes a fragment of an ActA protein. Each possibility represents a separate embodiment of the present invention.

In another embodiment of methods and compositions of the present invention, a PEST-like AA sequence is fused to the oncogene fragment.

In another embodiment, the PEST-like sequence in the first recombinant peptide is fused to the first fragment of the protein of interest. In another embodiment, the PEST-like sequence in the second recombinant peptide is fused to the second fragment of the protein of interest. In another embodiment, the PEST-like sequence in the third recombinant peptide is fused to the third fragment of the protein of interest. In another embodiment, the PEST-like sequence in the fourth recombinant peptide is fused to the fourth fragment of the protein of interest. In another embodiment, the PEST-like sequence in each recombinant peptide is fused to the fragment of the protein of interest. Each possibility represents a separate embodiment of the present invention.

In another embodiment, the first fragment of the protein of interest is embedded within the PEST-like sequence in the first recombinant peptide. In another embodiment, the second fragment of the protein of interest is embedded within the PEST-like sequence in the second recombinant peptide. In another embodiment, the third fragment of the protein of interest is embedded within the PEST-like sequence in the third recombinant peptide. In another embodiment, the fourth fragment of the protein of interest is embedded within the PEST-like sequence in the fourth recombinant peptide. In another embodiment, the fragment of the protein of interest of each recombinant peptide is embedded within the PEST-like sequence. Each possibility represents a separate embodiment of the present invention.

In another embodiment, the PEST-like AA sequence is KENSISSMAPPASPPASPKTPIEKKHADEIDK (SEQ ID NO: 22). In another embodiment, the PEST-like sequence is KENSISSMAPPASPPASPK (SEQ ID No: 23). In another embodiment, fusion of an antigen to any ΔLLO, including the PEST-like AA sequence, SEQ ID NO: 24, can enhance cell mediated and anti-tumor immunity of the antigen.

The PEST-like AA sequence has, in another embodiment, a sequence selected from SEQ ID NO: 25-31. In another embodiment, the PEST-like sequence is a PEST-like sequence from a Listeria ActA protein. In another embodiment, the PEST-like sequence is KTEEQPSEVNTGPR (SEQ ID NO: 25), KASVTDTSEGDLDSSMQSADESTPQPLK (SEQ ID NO: 26), KNEEVNASDFPPPPTDEELR (SEQ ID NO: 27), or RGGIPTSEEFSSLNSGDFTDDENSETTEEEIDR (SEQ ID NO: 28). In another embodiment, the PEST-like sequence is from Listeria seeligeri cytolysin, encoded by the Iso gene. In another embodiment, the PEST-like sequence is RSEVTISPAETPESPPATP (SEQ ID NO: 29). In another embodiment, the PEST-like sequence is from Streptolysin O protein of Streptococcus sp. In another embodiment, the PEST-like sequence is from Streptococcus pyogenes Streptolysin O, e.g. KQNTASTETTTTNEQPK (SEQ ID NO: 30) at AA 35-51. In another embodiment, the PEST-like sequence is from Streptococcus equisimilis Streptolysin O, e.g. KQNTANTETTTTNEQPK (SEQ ID NO: 31) at AA 38-54. In another embodiment, the PEST-like sequence is another PEST-like AA sequence derived from a prokaryotic organism.

PEST-like sequences of other prokaryotic organism are identified, in another embodiment, in accordance with methods such as described by, for example Rechsteiner and Rogers (1996, Trends Biochem. Sci. 21:267-271) for LM. Alternatively, PEST-like AA sequences from other prokaryotic organisms can also be identified based by this method. Other prokaryotic organisms wherein PEST-like AA sequences would be expected to include, but are not limited to, other Listeria species. In another embodiment, the PEST-like sequence is embedded within the antigenic protein. Thus, in another embodiment, “fusion” refers to an antigenic protein comprising an antigenic peptide and a PEST-like amino acid sequence linked at one end of the antigenic peptide. In another embodiment, the term refers to an antigenic protein comprising PEST-like amino acid sequence embedded within the antigenic peptide. Each possibility represents a separate embodiment of the present invention.

In another embodiment, the PEST-like sequence is identified using the PEST-find program. In another embodiment, a PEST-like sequence is defined as a hydrophilic stretch of at least 12 AA in length with a high local concentration of proline (P), aspartate (D), glutamate (E), serine (S), and/or threonine (T) residues. In another embodiment, a PEST-like sequence contains no positively charged AA, namely arginine (R), histidine (H) and lysine (K).

In another embodiment, identification of PEST motifs is achieved by an initial scan for positively charged AA R, H, and K within the specified protein sequence. All AA between the positively charged flanks are counted and only those motifs are considered further, which contain a number of AA equal to or higher than the window-size parameter. In another embodiment, a PEST-like sequence must contain at least 1 P, 1 D or E, and at least 1 S or T.

In another embodiment, the quality of a PEST motif is refined by means of a scoring parameter based on the local enrichment of critical AA as well as the motif's hydrophobicity. Enrichment of D, E, P, S and T is expressed in mass percent (w/w) and corrected for 1 equivalent of D or E, 1 of P and 1 of S or T. In another embodiment, calculation of hydrophobicity follows in principle the method of J. Kyte and R. F. Doolittle (Kyte, J and Dootlittle, R F. J. Mol. Biol. 157, 105 (1982). For simplified calculations, Kyte-Doolittle hydropathy indices, which originally ranged from −4.5 for arginine to +4.5 for isoleucine, are converted to positive integers, using the following linear transformation, which yielded values from 0 for arginine to 90 for isoleucine.

Hydropathy index=10*Kyte-Doolittle hydropathy index+45

In another embodiment, a potential PEST motif's hydrophobicity is calculated as the sum over the products of mole percent and hydrophobicity index for each AA species. The desired PEST score is obtained as combination of local enrichment term and hydrophobicity term as expressed by the following equation:

PEST score=0.55*DEPST−0.5*hydrophobicity index.

In another embodiment, “PEST-like sequence,” “PEST-like sequence peptide,” or “PEST-like sequence-containing peptide” refers to a peptide having a score of at least +5, using the above algorithm. In another embodiment, the term refers to a peptide having a score of at least 6. In another embodiment, the peptide has a score of at least 7. In another embodiment, the score is at least 8. In another embodiment, the score is at least 9. In another embodiment, the score is at least 10. In another embodiment, the score is at least 11. In another embodiment, the score is at least 12. In another embodiment, the score is at least 13. In another embodiment, the score is at least 14. In another embodiment, the score is at least 15. In another embodiment, the score is at least 16. In another embodiment, the score is at least 17. In another embodiment, the score is at least 18. In another embodiment, the score is at least 19. In another embodiment, the score is at least 20. In another embodiment, the score is at least 21. In another embodiment, the score is at least 22. In another embodiment, the score is at least 22. In another embodiment, the score is at least 24. In another embodiment, the score is at least 24. In another embodiment, the score is at least 25. In another embodiment, the score is at least 26. In another embodiment, the score is at least 27. In another embodiment, the score is at least 28. In another embodiment, the score is at least 29. In another embodiment, the score is at least 30. In another embodiment, the score is at least 32. In another embodiment, the score is at least 35. In another embodiment, the score is at least 38. In another embodiment, the score is at least 40. In another embodiment, the score is at least 45. Each possibility represents a separate embodiment of the present invention.

In another embodiment, the PEST-like sequence is identified using any other method or algorithm known in the art, e.g. the CaSPredictor (Garay-Malpartida H M, Occhiucci J M, Alves J, Belizario J E. Bioinformatics. 2005 June; 21 Suppl 1:i169-76). In another embodiment, the following method is used:

A PEST index is calculated for each stretch of appropriate length (e.g. a 30-35 AA stretch) by assigning a value of 1 to the AA Ser, Thr, Pro, Glu, Asp, Asn, or Gln. The coefficient value (CV) for each of the PEST residue is 1 and for each of the other AA (non-PEST) is 0.

Each method for identifying a PEST-like sequence represents a separate embodiment of the present invention.

“Fusion to a PEST-like sequence” refers, in another embodiment, to fusion to a protein fragment comprising a PEST-like sequence. In another embodiment, the term includes cases wherein the protein fragment comprises surrounding sequence other than the PEST-like sequence. In another embodiment, the protein fragment consists of the PEST-like sequence. Each possibility represents a separate embodiment of the present invention.

In another embodiment, fusion proteins of the present invention are prepared by subcloning of appropriate sequences, followed by expression of the resulting nucleotide. In another embodiment, subsequences are cloned and the appropriate subsequences cleaved using appropriate restriction enzymes. The fragments are then ligated, in another embodiment, to produce the desired DNA sequence. In another embodiment, DNA encoding the fusion protein is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The insert is then ligated into a plasmid. In another embodiment, a similar strategy is used to produce a protein wherein a tumor-associated protein fragment is embedded within a heterologous peptide.

In another embodiment, a recombinant polypeptide of the present invention is made by a process comprising the step of chemically conjugating a first polypeptide comprising a Her-2 fragment to a second polypeptide comprising a non-Her-2 AA sequence. In another embodiment, a Her-2 fragment is conjugated to a second polypeptide comprising the non-Her-2 AA sequence. In another embodiment, a peptide comprising a Her-2 fragment is conjugated to a non-Her-2 AA sequence. In another embodiment, a Her-2 fragment is conjugated to a non-Her-2 AA sequence. Each possibility represents a separate embodiment of the present invention.

In another embodiment, another method known in the art is utilized for conjugating the LLO sequence, ActA sequence, or PEST-like sequence to the Her-2 fragment. Methods for chemical conjugation of peptides to one another are well known in the art, and are described for, example, in (Biragyn, A and Kwak, L W (2001) Mouse models for lymphoma in “Current Protocols in Immunology” 20.6.1-20.6.30) and (Collawn, J. F. and Paterson, Y. (1989) Preparation of Anti-peptide antibodies. In Current Protocols in Molecular Biology. Supplement 6. Ed. F. M. Ausubel et. al. Greene Publishing/Wiley 11.14.1-11.15.3).

In another embodiment, glutaraldehyde is used for the conjugation.

In another embodiment, a fusion peptide of the present invention is synthesized using standard chemical peptide synthesis techniques. In another embodiment, the chimeric molecule is synthesized as a single contiguous polypeptide. In another embodiment, the LLO protein, ActA protein, or fragment thereof; and the Her-2 fragment are synthesized separately, then fused by condensation of the amino terminus of one molecule with the carboxyl terminus of the other molecule, thereby forming a peptide bond. In another embodiment, the ActA protein or LLO protein and Her-2 fragment are each condensed with one end of a peptide spacer molecule, thereby forming a contiguous fusion protein.

In another embodiment, the peptides and proteins of the present invention are prepared by solid-phase peptide synthesis (SPPS) as described by Stewart et al. in Solid Phase Peptide Synthesis, 2nd Edition, 1984, Pierce Chemical Company, Rockford, Ill.; or as described by Bodanszky and Bodanszky (The Practice of Peptide Synthesis, 1984, Springer-Verlag, New York). In another embodiment, a suitably protected AA residue is attached through its carboxyl group to a derivatized, insoluble polymeric support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial AA, and couple thereto of the carboxyl end of the next AA in the sequence of the desired peptide. This AA is also suitably protected. The carboxyl of the incoming AA can be activated to react with the N-terminus of the support-bound AA by formation into a reactive group such as formation into a carbodiimide, a symmetric acid anhydride or an “active ester” group such as hydroxybenzotriazole or pentafluorophenyl esters.

Examples of solid phase peptide synthesis methods include the BOC method which utilized tert-butyloxycarbonyl as the alpha-amino protecting group, and the FMOC method which utilizes 9-fluorenylmethyloxycarbonyl to protect the alpha-amino of the AA residues, both methods of which are well-known by those of skill in the art.

In another embodiment, incorporation of N- and/or C-blocking groups is achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups is achieved, in another embodiment, while the synthesized peptide is still attached to the resin, for instance by treatment with a suitable anhydride and nitrile. To incorporate an acetyl blocking group at the N-terminus, for instance, the resin coupled peptide can be treated with 20% acetic anhydride in acetonitrile. The N-blocked peptide product can then be cleaved from the resin, deprotected and subsequently isolated.

In another embodiment, the Her-2 fragment is conjugated directly to the LLO sequence, ActA sequence, or PEST-like sequence. In another embodiment, the Her-2 fragment is conjugated, either through a linker (spacer) to the LLO sequence, ActA sequence, or PEST-like sequence.

In another embodiment, the present invention provides a method of treating a tumor expressing a tumor-associated protein in a subject, comprising the steps of: (A) administering to the subject a vaccine comprising a first recombinant peptide, the first recombinant peptide comprising a first fragment of the tumor-associated protein; and subsequent to step (A), administering to the subject a vaccine comprising a second recombinant peptide, the second recombinant peptide comprising a second fragment of the tumor-associated protein, thereby treating a tumor expressing a tumor-associated protein in a subject.

In another embodiment, the present invention provides a method of suppressing a formation of a tumor expressing a tumor-associated protein in a subject, comprising the steps of: (A) administering to the subject a vaccine comprising a first recombinant peptide, the first recombinant peptide comprising a first fragment of the tumor-associated protein; and (B) subsequent to step (A), administering to the subject a vaccine comprising a second recombinant peptide, the second recombinant peptide comprising a second fragment of the tumor-associated protein, thereby suppressing a formation of a tumor expressing a tumor-associated protein in a subject.

In another embodiment, step (A) of methods of the present invention results in tumor regression. In another embodiment, step (A) results in a clinical endpoint. In another embodiment, the clinical endpoint is any clinical endpoint known in the art. In another embodiment, the subsequent step of a method of the present invention is not initiated until the clinical endpoint is achieved. In another embodiment, the subsequent step of a method of the present invention is not initiated as long as the clinical endpoint is maintained. Each possibility represents a separate embodiment of the present invention.

In another embodiment, step (A) results in the appearance of an escape mutation. As provided herein, CTL escape mutants can arise during active immunotherapy for cancer. In another embodiment, step (A) results in the appearance of multiple escape mutations. In another embodiment, the subsequent step of a method of the present invention is not initiated until an escape mutation is observed. In another embodiment, an escape mutation that is observed in or prior to a method of the present invention reduces a catalytic function of the tumor-associated protein. In another embodiment, the escape mutation reduces but does not abrogate the catalytic function. In another embodiment, the escape mutation does not reduce the catalytic function. In another embodiment, step (B) results in the appearance of an escape mutation, as described for step (A). In another embodiment, the subsequent step of a method of the present invention is not initiated until an escape mutation is observed. In another embodiment, the subsequent step of a method of the present invention is not initiated until a particular type of escape mutation is observed. In another embodiment, the escape mutation is in the catalytic domain of the tumor-associated protein. In another embodiment, the escape mutation is in a region of the catalytic domain not required for catalytic function. In another embodiment, the escape mutation is in a region of the catalytic domain required for catalytic function, but outside the active site. As provided herein, under the conditions utilized herein, the majority of mutations in IC1 mutations lay outside the ATP-binding domain, in regions likely to be less essential to the function of the kinase domain. Each possibility represents a separate embodiment of the present invention.

In another embodiment, the present invention provides a method of treating a tumor expressing a Her-2 protein in a subject, comprising the steps of:
(A) administering to the subject a first recombinant peptide, the first recombinant peptide comprising a first fragment of the Her-2 protein, wherein the first fragment is selected from: (1) a first intracellular fragment of the Her-2 protein, comprising the kinase domain of the Her-2 protein or a fragment of the kinase domain; (2) a second intracellular fragment of the Her-2 protein, wherein the second intracellular fragment either does not overlap with the first intracellular fragment or overlaps less than 20% with the first intracellular fragment; (3) a first extracellular fragment of the Her-2 protein, comprising the N-terminal half of the extracellular domain of the Her-2 protein or a fragment of the N-terminal half; and (4) a second extracellular fragment of the Her-2 protein, comprising the middle third of the extracellular domain of the Her-2 protein or a fragment of the middle third; and
(B) subsequent to step (A), administering to the subject a second recombinant peptide, the second recombinant peptide comprising a second fragment of the Her-2 protein, wherein the second fragment is selected from above fragments 1-4, but different from the first fragment, thereby treating a tumor expressing a Her-2 protein in a subject.

In another embodiment, the Her-2 fragments are selected from about amino acids (AA) 22-326, about AA 303-501, about AA 479-652, about AA 677-1081, and about AA 1020-1255.

In another embodiment of a methods of the present invention, a first vaccine comprising a first recombinant peptide is administered in step (A), and a second vaccine comprising a second recombinant peptide is administered in step (B).

In one embodiment, the Her-2 protein of methods and compositions of the present invention is a human Her-2 protein. In another embodiment, the Her-2 protein is a mouse Her-2 protein. In another embodiment, the Her-2 protein is a rat Her-2 protein. In another embodiment, the Her-2 protein is a primate Her-2 protein. In another embodiment, the Her-2 protein is a Her-2 protein of any other animal species known in the art. In another embodiment, the Her-2 protein is a variant of a Her-2 protein. In another embodiment, the Her-2 protein is a homologue of a Her-2 protein. Each possibility represents a separate embodiment of the present invention.

In another embodiment, the Her-2 protein is a rat Her-2 protein having the sequence:

(SEQ ID No: 32)

MIIMELAAWCRWGFLLALLPPGIAGTQVCTGTDMKLRLPASPETHLDMLR
HLYQGCQVVQGNLELTYVPANASLSFLQDIQEVQGYMLIAHNQVKRVPLQ
RLRIVRGTQLFEDKYALAVLDNRDPQDNVAASTPGRTPEGLRELQLRSLT
EILKGGVLIRGNPQLCYQDMVLWKDVFRKNNQLAPVDIDTNRSRACPPCA
PACKDNHCWGESPEDCQILTGTICTSGCARCKGRLPTDCCHEQCAAGCTG
PKHSDCLACLHFNHSGICELHCPALVTYNTDTFESMHNPEGRYTFGASCV
TTCPYNYLSTEVGSCTLVCPPNNQEVTAEDGTQRCEKCSKPCARVCYGLG
MEHLRGARAITSDNVQEFDGCKKIFGSLAFLPESFDGDPSSGIAPLRPEQ
LQVFETLEEITGYLYISAWPDSLRDLSVFQNLRIIRGRILHDGAYSLTLQ
GLGIHSLGLRSLRELGSGLALIHRNAHLCFVHTVPWDQLFRNPHQALLHS
GNRPEEDCGLEGLVCNSLCAHGHCWGPGPTQCVNCSHFLRGQECVEECRV
WKGLPREYVSDKRCLPCHPECQPQNSSETCFGSEADQCAACAHYKDSSSC
VARCPSGVKPDLSYMPIWKYPDEEGICQPCPINCTHSCVDLDERGCPAEQ
RASPVTFIIATVEGVLLFLILVVVVGILIKRRRQKIRKYTMRRLLQETEL
VEPLTPSGAMPNQAQMRILKETELRKVKVLGSGAFGTVYKGIWIPDGENV
KIPVAIKVLRENTSPKANKEILDEAYVMAGVGSPYVSRLLGICLTSTVQL
VTQLMPYGCLLDHVREHRGRLGSQDLLNWCVQIAKGMSYLEDVRLVHRDL
AARNVLVKSPNHVKITDFGLARLLDIDETEYHADGGKVPIKWMALESILR
RRFTHQSDVWSYGVTVWELMTFGAKPYDGIPAREIPDLLEKGERLPQPPI
CTIDVYMIMVKCWMIDSECRPRFRELVSEFSRMARDPQRFVVIQNEDLGP
SSPMDSTFYRSLLEDDDMGDLVDAEEYLVPQQGFFSPDPTPGTGSTAHRR
HRSSSTRSGGGELTLGLEPSEEGPPRSPLAPSEGAGSDVFDGDLAMGVTK
GLQSLSPHDLSPLQRYSEDPTLPLPPETDGYVAPLACSPQPEYVNQSEVQ
PQPPLTPEGPLPPVRPAGATLERPKTLSPGKNGVVKDVFAFGGAVENPEY
LVPREGTASPPHPSPAFSPAFDNLYYWDQNSSEQGPPPSNFEGTPTAENP
EYLGLDVPV.

In another embodiment, the Her-2 protein is a human Her-2 protein having the sequence:

(SEQ ID No: 33)

MELAALCRWGLLLALLPPGAASTQVCTGTDMKLRLPASPETHLDMLRHL
YQGCQVVQGNLELTYLPTNASLSFLQDIQEVQGYVLIAHNQVRQVPLQR
LRIVRGTQLFEDNYALAVLDNGDPLNNTTPVTGASPGGLRELQLRSLTE
ILKGGVLIQRNPQLCYQDTILWKDIFHKNNQLALTLIDTNRSRACHPCS
PMCKGSRCWGESSEDCQSLTRTVCAGGCARCKGPLPTDCCHEQCAAGCT
GPKHSDCLACLHFNHSGICELHCPALVTYNTDTFESMPNPEGRYTFGAS
CVTACPYNYLSTDVGSCTLVCPLHNQEVTAEDGTQRCEKCSKPCARVCY
GLGMEHLREVRAVTSANIQEFAGCKKIFGSLAFLPESFDGDPASNTAPL
QPEQLQVFETLEEITGYLYISAWPDSLPDLSVFQNLQVIRGRILHNGAY
SLTLQGLGISWLGLRSLRELGSGLALIHHNTHLCFVHTVPWDQLFRNPH
QALLHTANRPEDECVGEGLACHQLCARGHCWGPGPTQCVNCSQFLRGQE
CVEECRVLQGLPREYVNARHCLPCHPECQPQNGSVTCFGPEADQCVACA
HYKDPPFCVARCPSGVKPDLSYMPIWKFPDEEGACQPCPINCTHSCVDL
DDKGCPAEQRASPLTSIISAVVGILLVVVLGVVFGILIKRRQQKIRKYT
MRRLLQETELVEPLTPSGAMPNQAQMRILKETELRKVKVLGSGAFGTVY
KGIWIPDGENVKIPVAIKVLRENTSPKANKEILDEAYVMAGVGSPYVSR
LLGICLTSTVQLVTQLMPYGCLLDHVRENRGRLGSQDLLNWCMQIAKGM
SYLEDVRLVHRDLAARNVLVKSPNHVKITDFGLARLLDIDETEYHADGG
KVPIKWMALESILRRRFTHQSDVWSYGVTVWELMTFGAKPYDGIPAREI
PDLLEKGERLPQPPICTIDVYMIMVKCWMIDSECRPRFRELVSEFSRMA
RDPQRFVVIQNEDLGPASPLDSTFYRSLLEDDDMGDLVDAEEYLVPQQG
FFCPDPAPGAGGMVHHRHRSSSTRSGGGDLTLGLEPSEEEAPRSPLAPS
EGAGSDVFDGDLGMGAAKGLQSLPTHDPSPLQRYSEDPTVPLPSETDGY
VAPLTCSPQPEYVNQPDVRPQPPSPREGPLPAARPAGATLERPKTLSPG
KNGVVKDVFAFGGAVENPEYLTPQGGAAPQPHPPPAFSPAFDNLYYWDQ
DPPERGAPPSTFKGTPTAENPEYLGLDVPV.

In other embodiments, the Her-2 protein has a sequence set forth in GenBank Accession No. NM_{—}004448 or NM_{—}001005862. These Her-2 proteins have transmembrane (TM) regions spanning AA 653-675 and 623-645, respectively. The human Her-2 protein set forth in SEQ ID No: 33 has a TM region spanning 653-676. Thus, in another embodiment, the generation of Her-2 fragments corresponding to those of the present invention from variations of SEQ ID No: 32 such as these requires adjustment of the residue numbers defining the fragments, as described below.

In other embodiments, a Her-2 protein is a protein referred to as “HER-2/neu,” “Erbb2,” “v-erb-b2,” “c-erb-b2,” “neu,” or “cNeu.” Each possibility represents a separate embodiment of the present invention.

In another embodiment, the fragment of a Her-2 protein of methods and compositions of the present invention consists of about amino acid (AA) 20-326 (EC1 of Example 1). In another embodiment, the fragment consists of about AA 303-501 (EC2) of the Her-2 protein. In another embodiment, the fragment consists of about AA 479-655 (EC3) of the Her-2 protein. In another embodiment, the fragment of a Her-2 protein consists of about AA 690-1081 (IC1) of the Her-2 protein. In another embodiment, the fragment consists of about AA 1020-1255 (IC2) of the Her-2 protein. Each possibility represents a separate embodiment of the present invention.

The AA numbers and ranges listed above are based on the rat Her-2 sequence, for which the TM domain spans residues 656-689. In another embodiment, corresponding regions of other Her-2 proteins (e.g. Her-2 proteins from other species) are determined by aligning the TM domains of the other Her-2 proteins and adjusting the AA ranges. For example, for human Her-2 transcript variant 2, GenBank Accession No. NM_{—}001005862, the TM region spans AA 623-645. Thus, in this embodiment, the region of this protein corresponding to EC3 is about AA 446-622, determined by subtracting 33 from the AA numbers to account for the 33 AA difference in the extracellular border of the TM domain. Similarly, the region of this protein corresponding to IC1 is 646-1037, determined by subtracting 44 from the numbers to account for the 44 AA difference in the intracellular border of the TM domain. In another embodiment, corresponding regions of other Her-2 proteins are determined by alignment with the ends of the protein. Each possibility represents a separate embodiment of the present invention.

In another embodiment, the fragment is a fragment of the extracellular domain of the Her-2 protein. In another embodiment, the fragment consists of about one-third to one-half of the extracellular domain of the Her-2 protein. In another embodiment, the fragment consists of about one-tenth to one-fifth thereof. In another embodiment, the fragment consists of about one-fifth to one-fourth thereof. In another embodiment, the fragment consists of about one-fourth to one-third thereof. In another embodiment, the fragment consists of about one-third to one-half thereof. In another embodiment, the fragment consists of about one-half to three quarters thereof. In another embodiment, the fragment consists of about three quarters to the entire extracellular domain. In another embodiment, the fragment consists of about 5-10% thereof. In another embodiment, the fragment consists of about 10-15% thereof. In another embodiment, the fragment consists of about 15-20% thereof. In another embodiment, the fragment consists of about 20-25% thereof. In another embodiment, the fragment consists of about 25-30% thereof. In another embodiment, the fragment consists of about 30-35% thereof. In another embodiment, the fragment consists of about 35-40% thereof. In another embodiment, the fragment consists of about 45-50% thereof. In another embodiment, the fragment consists of about 50-55% thereof. In another embodiment, the fragment consists of about 55-60% thereof. In another embodiment, the fragment consists of about 5-15% thereof. In another embodiment, the fragment consists of about 10-20% thereof. In another embodiment, the fragment consists of about 15-25% thereof. In another embodiment, the fragment consists of about 20-30% thereof. In another embodiment, the fragment consists of about 25-35% thereof. In another embodiment, the fragment consists of about 30-40% thereof. In another embodiment, the fragment consists of about 35-45% thereof. In another embodiment, the fragment consists of about 45-55% thereof. In another embodiment, the fragment consists of about 50-60% thereof. In another embodiment, the fragment consists of about 55-65% thereof. In another embodiment, the fragment consists of about 60-70% thereof. In another embodiment, the fragment consists of about 65-75% thereof. In another embodiment, the fragment consists of about 70-80% thereof. In another embodiment, the fragment consists of about 5-20% thereof. In another embodiment, the fragment consists of about 10-25% thereof. In another embodiment, the fragment consists of about 15-30% thereof. In another embodiment, the fragment consists of about 20-35% thereof. In another embodiment, the fragment consists of about 25-40% thereof. In another embodiment, the fragment consists of about 30-45% thereof. In another embodiment, the fragment consists of about 35-50% thereof. In another embodiment, the fragment consists of about 45-60% thereof. In another embodiment, the fragment consists of about 50-65% thereof. In another embodiment, the fragment consists of about 55-70% thereof. In another embodiment, the fragment consists of about 60-75% thereof. In another embodiment, the fragment consists of about 65-80% thereof. In another embodiment, the fragment consists of about 70-85% thereof. In another embodiment, the fragment consists of about 75-90% thereof. In another embodiment, the fragment consists of about 80-95% thereof. In another embodiment, the fragment consists of about 85-100% thereof. In another embodiment, the fragment consists of about 5-25% thereof. In another embodiment, the fragment consists of about 10-30% thereof. In another embodiment, the fragment consists of about 15-35% thereof. In another embodiment, the fragment consists of about 20-40% thereof. In another embodiment, the fragment consists of about 30-50% thereof. In another embodiment, the fragment consists of about 40-60% thereof. In another embodiment, the fragment consists of about 50-70% thereof. In another embodiment, the fragment consists of about 60-80% thereof. In another embodiment, the fragment consists of about 70-90% thereof. In another embodiment, the fragment consists of about 80-100% thereof. In another embodiment, the fragment consists of about 5-35% thereof. In another embodiment, the fragment consists of about 10-40% thereof. In another embodiment, the fragment consists of about 15-45% thereof. In another embodiment, the fragment consists of about 20-50% thereof. In another embodiment, the fragment consists of about 30-60% thereof. In another embodiment, the fragment consists of about 40-70% thereof. In another embodiment, the fragment consists of about 50-80% thereof. In another embodiment, the fragment consists of about 60-90% thereof. In another embodiment, the fragment consists of about 70-100% thereof. In another embodiment, the fragment consists of about 5-45% thereof. In another embodiment, the fragment consists of about 10-50% thereof. In another embodiment, the fragment consists of about 20-60% thereof. In another embodiment, the fragment consists of about 30-70% thereof. In another embodiment, the fragment consists of about 40-80% thereof. In another embodiment, the fragment consists of about 50-90% thereof. In another embodiment, the fragment consists of about 60-100% thereof. In another embodiment, the fragment consists of about 5-55% thereof. In another embodiment, the fragment consists of about 10-60% thereof. In another embodiment, the fragment consists of about 20-70% thereof. In another embodiment, the fragment consists of about 30-80% thereof. In another embodiment, the fragment consists of about 40-90% thereof. In another embodiment, the fragment consists of about 50-100% thereof. In another embodiment, the fragment consists of about 5-65% thereof. In another embodiment, the fragment consists of about 10-70% thereof. In another embodiment, the fragment consists of about 20-80% thereof. In another embodiment, the fragment consists of about 30-90% thereof. In another embodiment, the fragment consists of about 40-100% thereof. In another embodiment, the fragment consists of about 5-75% thereof. In another embodiment, the fragment consists of about 10-80% thereof. In another embodiment, the fragment consists of about 20-90% thereof. In another embodiment, the fragment consists of about 30-100% thereof. In another embodiment, the fragment consists of about 10-90% thereof. In another embodiment, the fragment consists of about 20-100% thereof. In another embodiment, the fragment consists of about 10-100% thereof.

In another embodiment, the fragment consists of about 5% of the extracellular domain. In another embodiment, the fragment consists of about 6% thereof. In another embodiment, the fragment consists of about 8% thereof. In another embodiment, the fragment consists of about 10% thereof. In another embodiment, the fragment consists of about 12% thereof. In another embodiment, the fragment consists of about 15% thereof. In another embodiment, the fragment consists of about 18% thereof. In another embodiment, the fragment consists of about 20% thereof. In another embodiment, the fragment consists of about 25% thereof. In another embodiment, the fragment consists of about 30% thereof. In another embodiment, the fragment consists of about 35% thereof. In another embodiment, the fragment consists of about 40% thereof. In another embodiment, the fragment consists of about 45% thereof. In another embodiment, the fragment consists of about 50% thereof. In another embodiment, the fragment consists of about 55% thereof. In another embodiment, the fragment consists of about 60% thereof. In another embodiment, the fragment consists of about 65% thereof. In another embodiment, the fragment consists of about 70% thereof. In another embodiment, the fragment consists of about 75% thereof. In another embodiment, the fragment consists of about 80% thereof. In another embodiment, the fragment consists of about 85% thereof. In another embodiment, the fragment consists of about 90% thereof. In another embodiment, the fragment consists of about 95% thereof. In another embodiment, the fragment consists of about 100% thereof. Each possibility represents a separate embodiment of the present invention.

In another embodiment, the fragment is a fragment of the intracellular domain of the Her-2 protein. In one embodiment, the fragment is from about one-third to one-half of the intracellular domain. In another embodiment, the fragment of the intracellular domain is any of the amounts, fractions, or ranges listed above for the extracellular domain. Each possibility represents a separate embodiment of the present invention.

In another embodiment, the fragment of a Her-2 protein of methods and compositions of the present invention does not include a signal sequence thereof. In one embodiment, omission of the signal sequence enables the Her-2 fragment to be successfully expressed in Listeria, due the high hydrophobicity of the signal sequence. In another embodiment, a Her-2 fragment including the signal sequence is expressed in by vector other than Listeria, in methods and compositions of the present invention. Each possibility represents a separate embodiment of the present invention.

In another embodiment, the fragment of a Her-2 protein of methods and compositions of the present invention does not include a TM domain thereof. In one embodiment, omission of the TM enables the Her-2 fragment to be successfully expressed in Listeria, due the high hydrophobicity of the TM. In another embodiment, a Her-2 fragment including the TM domain is expressed in by vector other than Listeria, in methods and compositions of the present invention. Each possibility represents a separate embodiment of the present invention.

In another embodiment, the present invention provides a method of suppressing a formation of a tumor expressing a Her-2 protein in a subject, comprising the steps of:
A. administering to the subject a first recombinant peptide, the first recombinant peptide comprising a first fragment of the Her-2 protein, wherein the first fragment is selected from: (1) a first intracellular fragment of the Her-2 protein, comprising the kinase domain of the Her-2 protein or a fragment of the kinase domain; (2) a second intracellular fragment of the Her-2 protein, wherein the second intracellular fragment either does not overlap with the first intracellular fragment or overlaps less than 20% with the first intracellular fragment; (3) a first extracellular fragment of the Her-2 protein, comprising the N-terminal half of the extracellular domain of the Her-2 protein or a fragment of the N-terminal half; and (4) a second extracellular fragment of the Her-2 protein, comprising the middle third of the extracellular domain of the Her-2 protein or a fragment of the middle third; and
(B) subsequent to step (A), administering to the subject a second recombinant peptide, the second recombinant peptide comprising a second fragment of the Her-2 protein, wherein the second fragment is selected from above fragments 1-4, but different from the first fragment, thereby suppressing a formation of a tumor expressing a Her-2 protein in a subject.

In another embodiment, a method of the present invention further comprises the step of, subsequent to step (B), administering to the subject a vaccine comprising a third recombinant peptide, the third recombinant peptide comprising a third fragment of the Her-2 protein, wherein the third fragment is selected from above fragments 1-4, but is different from the first fragment and the second fragment.

In another embodiment, an LLO fragment in the first recombinant peptide is fused to the first Her-2 fragment. In another embodiment, an LLO peptide in the second recombinant peptide is fused to the second Her-2 fragment. In another embodiment, an LLO peptide in the third recombinant peptide is fused to the third Her-2 fragment. In another embodiment, an LLO peptide in the fourth recombinant peptide is fused to the fourth Her-2 fragment. In another embodiment, the Her-2 fragment in each recombinant peptide is fused to an LLO peptide. Each possibility represents a separate embodiment of the present invention.

In another embodiment, a Her-2 fragment of methods and compositions of the present invention is embedded within an LLO peptide. In another embodiment, the first Her-2 fragment is embedded within the LLO peptide in the first recombinant peptide. In another embodiment, the second Her-2 fragment is embedded within the LLO peptide in the second recombinant peptide. In another embodiment, the third Her-2 fragment is embedded within the LLO peptide in the third recombinant peptide. In another embodiment, the fourth Her-2 fragment is embedded within the LLO peptide in the fourth recombinant peptide. In another embodiment, the Her-2 fragment in each recombinant peptide is embedded within an LLO peptide. Each possibility represents a separate embodiment of the present invention.

In another embodiment, an LLO fragment in the first recombinant peptide is fused to the first Her-2 fragment. In another embodiment, an ActA peptide or PEST-like sequence in the second recombinant peptide is fused to the second Her-2 fragment. In another embodiment, an ActA peptide or PEST-like sequence in the third recombinant peptide is fused to the third Her-2 fragment. In another embodiment, an ActA peptide or PEST-like sequence in the fourth recombinant peptide is fused to the fourth Her-2 fragment. In another embodiment, the Her-2 fragment in each recombinant peptide is fused to an ActA peptide or PEST-like sequence. Each possibility represents a separate embodiment of the present invention.

In another embodiment, a Her-2 fragment of methods and compositions of the present invention is embedded within an ActA peptide or PEST-like sequence. In another embodiment, the first Her-2 fragment is embedded within the ActA peptide or PEST-like sequence in the first recombinant peptide. In another embodiment, the second Her-2 fragment is embedded within the ActA peptide or PEST-like sequence in the second recombinant peptide. In another embodiment, the third Her-2 fragment is embedded within the ActA peptide or PEST-like sequence in the third recombinant peptide. In another embodiment, the fourth Her-2 fragment is embedded within the ActA peptide or PEST-like sequence in the fourth recombinant peptide. In another embodiment, the Her-2 fragment in each recombinant peptide is embedded within an ActA peptide or PEST-like sequence. Each possibility represents a separate embodiment of the present invention.

In another embodiment, the present invention provides a recombinant peptide comprising a fragment of a Her-2 protein. In another embodiment, the fragment is ALCRWGLLL (SEQ ID No: 11). In another embodiment, the fragment is HLYQGCQVV (SEQ ID No: 12). In another embodiment, the fragment is LTYLPTNASLSFLQD (SEQ ID No: 13). In another embodiment, the fragment is TYLPTNASL (SEQ ID No: 14). In another embodiment, the fragment is QLFEDNYAL (SEQ ID No: 15). In another embodiment, the fragment is KIGFSLAFL (SEQ ID No: 16). In another embodiment, the fragment is KIFGSLAFLPESFDGDPA (SEQ ID No: 17). In another embodiment, the fragment is PLQPEQLQV (SEQ ID No: 35). In another embodiment, the fragment is TLEEITGYL (SEQ ID No: 36). In another embodiment, the fragment is ILHNGAYSL (SEQ ID No: 37). In another embodiment, the fragment is ALIHHNTHL (SEQ ID No: 38). In another embodiment, the fragment is KPDLSYMPIWKFPDE (SEQ ID No: 39). In another embodiment, the fragment is PLTSIISAV (SEQ ID No: 40). In another embodiment, the fragment is IISAVVGIL (SEQ ID No: 41). In another embodiment, the fragment is RRLLQETELVEPLTPS (SEQ ID No: 42). In another embodiment, the fragment is RLLQETELV (SEQ ID No: 43). In another embodiment, the fragment is VLRENTSPK (SEQ ID No: 44). In another embodiment, the fragment is KEILDEAYVMAGVGSPYVS (SEQ ID No: 45). In another embodiment, the fragment is VMAGVGSPYV (SEQ ID No: 46). In another embodiment, the fragment is GSPYVSRLLGICL (SEQ ID No: 47). In another embodiment, the fragment is SPYVSRLLGICLT (SEQ ID No: 48). In another embodiment, the fragment is PYVSRLLGI (SEQ ID No: 49). In another embodiment, the fragment is LLGICLTSTV (SEQ ID No: 50). In another embodiment, the fragment is CLTSTVQLV (SEQ ID No: 51). In another embodiment, the fragment is QLMPYGCLL (SEQ ID No: 52). In another embodiment, the fragment is LLNWCMQIAKGMSYL (SEQ ID No: 53). In another embodiment, the fragment is YLEDVRLV (SEQ ID No: 54). In another embodiment, the fragment is VLVKSPNHV (SEQ ID No: 55). In another embodiment, the fragment is KVPIKWMALESILRRRF (SEQ ID No: 56). In another embodiment, the fragment is YMIMVKCWMI (SEQ ID No: 57). In another embodiment, the fragment is ELVSEFSRM (SEQ ID No: 58). In another embodiment, the fragment is ELVSEFSRMARDPQ (SEQ ID No: 59). In another embodiment, the fragment is YLVPQQGFFC (SEQ ID No: 60).

In another embodiment, the recombinant peptide consists of a Her-2 fragment containing a peptide listed in Table 3 herein. In another embodiment, the recombinant peptide consists of an MHC molecule-binding peptide listed in Table 3 herein. In another embodiment, the recombinant peptide consists of an MHC class I molecule-binding peptide from Table 3. In another embodiment, the recombinant peptide consists of an MHC class II molecule-binding peptide from Table 3 herein.

In another embodiment, the fragment contains a mutated residue listed in Table 1 herein. In another embodiment, the fragment is an MHC molecule-binding peptide containing a mutated residue listed in Table 1 herein. In another embodiment, the fragment is an MHC class I molecule-binding peptide containing a mutated residue from Table 1. In another embodiment, the fragment is an MHC class II molecule-binding peptide containing a mutated residue from Table 1 herein.

In another embodiment, the recombinant peptide consists of HLYQGCQVV (SEQ ID No: 12). In another embodiment, the recombinant peptide consists of LTYLPTNASLSFLQD (SEQ ID No: 13). In another embodiment, the recombinant peptide consists of TYLPTNASL (SEQ ID No: 14). In another embodiment, the recombinant peptide consists of QLFEDNYAL (SEQ ID No: 15). In another embodiment, the recombinant peptide consists of KIGFSLAFL (SEQ ID No: 16). In another embodiment, the recombinant peptide consists of KIFGSLAFLPESFDGDPA (SEQ ID No: 17). In another embodiment, the recombinant peptide consists of PLQPEQLQV (SEQ ID No: 35). In another embodiment, the recombinant peptide consists of TLEEITGYL (SEQ ID No: 36). In another embodiment, the recombinant peptide consists of ILHNGAYSL (SEQ ID No: 37). In another embodiment, the recombinant peptide consists of ALIHHNTHL (SEQ ID No: 38). In another embodiment, the recombinant peptide consists of KPDLSYMPIWKFPDE (SEQ ID No: 39). In another embodiment, the recombinant peptide consists of PLTSIISAV (SEQ ID No: 40). In another embodiment, the recombinant peptide consists of IISAVVGIL (SEQ ID No: 41). In another embodiment, the recombinant peptide consists of RRLLQETELVEPLTPS (SEQ ID No: 42). In another embodiment, the recombinant peptide consists of RLLQETELV (SEQ ID No: 43). In another embodiment, the recombinant peptide consists of VLRENTSPK (SEQ ID No: 44). In another embodiment, the recombinant peptide consists of KEILDEAYVMAGVGSPYVS (SEQ ID No: 45). In another embodiment, the recombinant peptide consists of VMAGVGSPYV (SEQ ID No: 46). In another embodiment, the recombinant peptide consists of GSPYVSRLLGICL (SEQ ID No: 47). In another embodiment, the recombinant peptide consists of SPYVSRLLGICLT (SEQ ID No: 48). In another embodiment, the recombinant peptide consists of PYVSRLLGI (SEQ ID No: 49). In another embodiment, the recombinant peptide consists of LLGICLTSTV (SEQ ID No: 50). In another embodiment, the recombinant peptide consists of CLTSTVQLV (SEQ ID No: 51). In another embodiment, the recombinant peptide consists of QLMPYGCLL (SEQ ID No: 52). In another embodiment, the recombinant peptide consists of LLNWCMQIAKGMSYL (SEQ ID No: 53). In another embodiment, the recombinant peptide consists of YLEDVRLV (SEQ ID No: 54). In another embodiment, the recombinant peptide consists of VLVKSPNHV (SEQ ID No: 55). In another embodiment, the recombinant peptide consists of KVPIKWMALESILRRRF (SEQ ID No: 56). In another embodiment, the recombinant peptide consists of YMIMVKCWMI (SEQ ID No: 57). In another embodiment, the recombinant peptide consists of ELVSEFSRM (SEQ ID No: 58). In another embodiment, the recombinant peptide consists of ELVSEFSRMARDPQ (SEQ ID No: 59). In another embodiment, the recombinant peptide consists of YLVPQQGFFC (SEQ ID No: 60).

In another embodiment, the recombinant peptide consists of a Her-2 fragment containing a mutated residue listed in Table 1 herein. In another embodiment, the recombinant peptide consists of an MHC molecule-binding peptide containing a mutated residue listed in Table 1 herein. In another embodiment, the recombinant peptide consists of an MHC class I molecule-binding peptide containing a mutated residue from Table 1. In another embodiment, the recombinant peptide consists of an MHC class II molecule-binding peptide containing a mutated residue from Table herein.

In another embodiment, the recombinant peptide comprises a mutation. In another embodiment, the mutation is 740A. In another embodiment, the mutation is 764D. In another embodiment, the mutation is Y786A. In another embodiment, the mutation is deletion of L801. In another embodiment, the mutation is L874R. In another embodiment, the mutation is V978L. In another embodiment, the mutation is a mutation enumerated in Table 1 herein. In another embodiment, the recombinant peptide consists of an MHC class I molecule-binding peptide containing one of the above mutations. Each possibility represents a separate embodiment of the present invention.

In another embodiment, an LLO peptide is fused to a Her-2 fragment in a peptide of methods and compositions of the present invention. In another embodiment, an ActA peptide is fused to a Her-2 fragment in a peptide of methods and compositions of the present invention. In another embodiment, a PEST-like sequence is fused to a Her-2 fragment in a peptide of methods and compositions of the present invention. Each possibility represents a separate embodiment of the present invention.

In another embodiment, a Her-2 fragment of methods and compositions of the present invention is embedded within an LLO peptide. In another embodiment, a Her-2 fragment of methods and compositions of the present invention is embedded within an ActA peptide. In another embodiment, a Her-2 fragment of methods and compositions of the present invention is embedded within a PEST-like sequence. Each possibility represents a separate embodiment of the present invention.

Each recombinant peptide and type of recombinant peptide represents a separate embodiment of the present invention.

In another embodiment, the present invention provides a recombinant vaccine vector encoding a recombinant polypeptide of the present invention.

In another embodiment, the present invention provides a recombinant nucleotide molecule encoding a recombinant polypeptide of the present invention.

In another embodiment, the present invention provides a vaccine comprising a recombinant nucleotide molecule of the present invention and an adjuvant.

In another embodiment, the present invention provides a recombinant vaccine vector comprising a recombinant nucleotide molecule of the present invention.

In another embodiment, the present invention provides a recombinant Listeria strain comprising a recombinant polypeptide of the present invention.

In another embodiment, the present invention provides a recombinant Listeria strain comprising a recombinant nucleotide molecule of the present invention.

The recombinant Listeria strain of methods and compositions of the present invention is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art.

In another embodiment the Listeria strain is attenuated by deletion of a gene. In another embodiment the Listeria strain is attenuated by deletion of more than 1 gene. In another embodiment the Listeria strain is attenuated by deletion or inactivation of a gene. In another embodiment the Listeria strain is attenuated by deletion or inactivation of more than 1 gene.

In another embodiment, the gene that is mutated is hly. In another embodiment, the gene that is mutated is actA. In another embodiment, the gene that is mutated is plc A. In another embodiment, the gene that is mutated is plcB. In another embodiment, the gene that is mutated is mpl. In another embodiment, the gene that is mutated is inl A. In another embodiment, the gene that is mutated is inlB. In another embodiment, the gene that is mutated is bsh.

In another embodiment, the Listeria strain is an auxotrophic mutant. In another embodiment, the Listeria strain is deficient in a gene encoding a vitamin synthesis gene. In another embodiment, the Listeria strain is deficient in a gene encoding pantothenic acid synthase.

In another embodiment, the Listeria strain is deficient in an AA metabolism enzyme. In another embodiment the Listeria strain is deficient in a D-glutamic acid synthase gene. In another embodiment the Listeria strain is deficient in the dat gene. In another embodiment the Listeria strain is deficient in the dga gene. In another embodiment the Listeria strain is deficient in a gene involved in the synthesis of diaminopimelic acid. CysK. In another embodiment, the gene is vitamin-B12 independent methionine synthase. In another embodiment, the gene is trpA. In another embodiment, the gene is trpB. In another embodiment, the gene is trpE. In another embodiment, the gene is asnB. In another embodiment, the gene is gltD. In another embodiment, the gene is gltB. In another embodiment, the gene is leuA. In another embodiment, the gene is argG. In another embodiment, the gene is thrC.

In another embodiment, the Listeria strain is deficient in a synthase gene. In another embodiment, the gene is an AA synthesis gene. In another embodiment, the gene is folP. In another embodiment, the gene is dihydrouridine synthase family protein. In another embodiment, the gene is ispD. In another embodiment, the gene is ispF. In another embodiment, the gene is phosphoenolpyruvate synthase. In another embodiment, the gene is hisF. In another embodiment, the gene is his H. In another embodiment, the gene is fliI. In another embodiment, the gene is ribosomal large subunit pseudouridine synthase. In another embodiment, the gene is ispD. In another embodiment, the gene is bifunctional GMP synthase/glutamine amidotransferase protein. In another embodiment, the gene is cobS. In another embodiment, the gene is cobB. In another embodiment, the gene is cbiD. In another embodiment, the gene is uroporphyrin-III C-methyltransferase/uroporphyrinogen-III synthase. In another embodiment, the gene is cobQ. In another embodiment, the gene is uppS. In another embodiment, the gene is truB. In another embodiment, the gene is dxs. In another embodiment, the gene is mvaS. In another embodiment, the gene is dapA. In another embodiment, the gene is ispG. In another embodiment, the gene is folC. In another embodiment, the gene is citrate synthase. In another embodiment, the gene is argJ. In another embodiment, the gene is 3-deoxy-7-phosphoheptulonate synthase. In another embodiment, the gene is indole-3-glycerol-phosphate synthase. In another embodiment, the gene is anthranilate synthase/glutamine amidotransferase component. In another embodiment, the gene is menB. In another embodiment, the gene is menaquinone-specific isochorismate synthase. In another embodiment, the gene is phosphoribosylformylglycinamidine synthase I or II. In another embodiment, the gene is phosphoribosylaminoimidazole-succinocarboxamide synthase. In another embodiment, the gene is carB. In another embodiment, the gene is carA. In another embodiment, the gene is thyA. In another embodiment, the gene is mgsA. In another embodiment, the gene is aroB. In another embodiment, the gene is hepB. In another embodiment, the gene is riuB. In another embodiment, the gene is ilvB. In another embodiment, the gene is ilvN. In another embodiment, the gene is alsS. In another embodiment, the gene is fabF. In another embodiment, the gene is fabH. In another embodiment, the gene is pseudouridine synthase. In another embodiment, the gene is pyrG. In another embodiment, the gene is truA. In another embodiment, the gene is pabB. In another embodiment, the gene is an atp synthase gene (e.g. atpC, atpD-2, aptG, atpA-2, etc).

In another embodiment, the gene is phoP. In another embodiment, the gene is aroA aroC. In another embodiment, the gene is aroD. In another embodiment, the gene is plcB.

In another embodiment, the Listeria strain is deficient in a peptide transporter. In another embodiment, the gene is ABC transporter/ATP-binding/permease protein. In another embodiment, the gene is oligopeptide ABC transporter/oligopeptide-binding protein. In another embodiment, the gene is oligopeptide ABC transporter/permease protein. In another embodiment, the gene is zinc ABC transporter/zinc-binding protein. In another embodiment, the gene is sugar ABC transporter. In another embodiment, the gene is phosphate transporter. In another embodiment, the gene is ZIP zinc transporter. In another embodiment, the gene is drug resistance transporter of the EmrB/QacA family. In another embodiment, the gene is sulfate transporter. In another embodiment, the gene is proton-dependent oligopeptide transporter. In another embodiment, the gene is magnesium transporter. In another embodiment, the gene is formate/nitrite transporter. In another embodiment, the gene is spermidine/putrescine ABC transporter. In another embodiment, the gene is Na/Pi-cotransporter. In another embodiment, the gene is sugar phosphate transporter. In another embodiment, the gene is glutamine ABC transporter. In another embodiment, the gene is major facilitator family transporter. In another embodiment, the gene is glycine betaine/L-proline ABC transporter. In another embodiment, the gene is molybdenum ABC transporter. In another embodiment, the gene is techoic acid ABC transporter. In another embodiment, the gene is cobalt ABC transporter. In another embodiment, the gene is ammonium transporter. In another embodiment, the gene is amino acid ABC transporter. In another embodiment, the gene is cell division ABC transporter. In another embodiment, the gene is manganese ABC transporter. In another embodiment, the gene is iron compound ABC transporter. In another embodiment, the gene is maltose/maltodextrin ABC transporter. In another embodiment, the gene is drug resistance transporter of the Bcr/CflA family.

In another embodiment, the gene is a subunit of one of the above proteins.

Each Listeria strain and type thereof represents a separate embodiment of the present invention.

In another embodiment, the recombinant Listeria of methods and compositions of the present invention is stably transformed with a construct encoding an antigen or an LLO-antigen fusion. In one embodiment, the construct contains a polylinker to facilitate further subcloning. Several techniques for producing recombinant Listeria are known; each technique represents a separate embodiment of the present invention.

In another embodiment, the construct or heterologous gene is integrated into the Listerial chromosome using homologous recombination. Techniques for homologous recombination are well known in the art, and are described, for example, in Frankel, F R, Hegde, S, Lieberman, J, and Y Paterson. Induction of a cell-mediated immune response to HIV gag using Listeria monocytogenes as a live vaccine vector. J. Immunol. 155: 4766-4774. 1995; Mata, M, Yao, Z, Zubair, A, Syres, K and Y Paterson, Evaluation of a recombinant Listeria monocytogenes expressing an HIV protein that protects mice against viral challenge. Vaccine 19:1435-45, 2001; Boyer, J D, Robinson, T M, Maciag, P C, Peng, X, Johnson, R S, Pavlakis, G, Lewis, M G, Shen, A, Siliciano, R, Brown, C R, Weiner, D, and Y Paterson. DNA prime Listeria boost induces a cellular immune response to SIV antigens in the Rhesus Macaque model that is capable of limited suppression of SIV239 viral replication. Virology. 333: 88-101, 2005. In another embodiment, homologous recombination is performed as described in U.S. Pat. No. 6,855,320. In another embodiment, a temperature sensitive plasmid is used to select the recombinants. Each technique represents a separate embodiment of the present invention.

In another embodiment, the construct or heterologous gene is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed; the position in the genome where the foreign gene has been inserted is unknown.

In another embodiment, the construct or heterologous gene is integrated into the Listerial chromosome using phage integration sites (Lauer P, Chow M Y et al, Construction, characterization, and use of two LM site-specific phage integration vectors. J Bacteriol 2002; 184(15): 4177-86). In another embodiment, an integrase gene and attachment site of a bacteriophage (e.g. U153 or PSA listeriophage) is used to insert the heterologous gene into the corresponding attachment site, which is, in another embodiment, any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. Each possibility represents a separate embodiment of the present invention.

In another embodiment, the construct is carried by the Listeria strain on a plasmid. An LM vector that expresses an E7 fusion protein has also been constructed via this technique. Lm-GG/E7 was made by complementing a prfA-deletion mutant with a plasmid containing a copy of the prfA gene and a copy of the E7 gene fused to a form of the LLO (hly) gene truncated to eliminate the hemolytic activity of the enzyme, as described in U.S. Pat. No. 6,565,852. Functional LLO is maintained by the organism via the endogenous chromosomal copy of hly.

In another embodiment, a recombinant peptide of the present invention is fused to a Listerial protein, such as PI-PLC, or a construct encoding same. In another embodiment, a signal sequence of a secreted Listerial protein such as hemolysin, ActA, or a phospholipase is fused to the antigen-encoding gene. In another embodiment, a signal sequence of the recombinant vaccine vector is used. In another embodiment, a signal sequence functional in the recombinant vaccine vector is used. Each possibility represents a separate embodiment of the present invention.

In another embodiment, the construct is contained in the Listeria strain in an episomal fashion. In another embodiment, the foreign antigen is expressed from a vector harbored by the recombinant Listeria strain.

Each method of expression in Listeria represents a separate embodiment of the present invention.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. Methods for passaging a recombinant Listeria strain through an animal host are well known in the art, and are described, for example, in U.S. patent application Ser. No. 10/541,614. Each possibility represents a separate embodiment of the present invention.

In another embodiment, the recombinant Listeria strain utilized in methods of the present invention has been stored in a frozen cell bank. In another embodiment, the recombinant Listeria strain has been stored in a lyophilized cell bank. Each possibility represents a separate embodiment of the present invention.

In another embodiment, the cell bank of methods and compositions of the present invention is a master cell bank. In another embodiment, the cell bank is a working cell bank. In another embodiment, the cell bank is Good Manufacturing Practice (GMP) cell bank. In another embodiment, the cell bank is intended for production of clinical-grade material. In another embodiment, the cell bank conforms to regulatory practices for human use. In another embodiment, the cell bank is any other type of cell bank known in the art. Each possibility represents a separate embodiment of the present invention.

“Good Manufacturing Practices” are defined, in another embodiment, by (21 CFR 210-211) of the United States Code of Federal Regulations. In another embodiment, “Good Manufacturing Practices” are defined by other standards for production of clinical-grade material or for human consumption; e.g. standards of a country other than the United States. Each possibility represents a separate embodiment of the present invention.

In another embodiment, a recombinant Listeria strain utilized in methods of the present invention is from a batch of vaccine doses.

In another embodiment, a recombinant Listeria strain utilized in methods of the present invention is from a frozen stock produced by a method disclosed herein.

In another embodiment, a recombinant Listeria strain utilized in methods of the present invention is from a lyophilized stock produced by a method disclosed herein. Methods for lyophilizing recombinant Listeria strains are well known in the art, and are described, for example, in PCT International Patent Application Publication No. WO 2007/061848. Each method represents a separate embodiment of the present invention.

In another embodiment, a cell bank, frozen stock, or batch of vaccine doses of the present invention is cryopreserved by a method that comprises growing a culture of the Listeria strain in a nutrient media, freezing the culture in a solution comprising glycerol, and storing the Listeria strain at below −20 degrees Celsius. In another embodiment, the temperature is about −70 degrees Celsius. In another embodiment, the temperature is about −70-−80 degrees Celsius.

In another embodiment, a cell bank, frozen stock, or batch of vaccine doses of the present invention is cryopreserved by a method that comprises growing a culture of the Listeria strain in a defined media of the present invention (as described below), freezing the culture in a solution comprising glycerol, and storing the Listeria strain at below −20 degrees Celsius. In another embodiment, the temperature is about −70 degrees Celsius. In another embodiment, the temperature is about −70-−80 degrees Celsius. Methods for cryopreservation of recombinant Listeria strains are well known in the art, and are described, for example, in PCT International Patent Application Publication No. WO 2007/061848. Each method represents a separate embodiment of the present invention.

In another embodiment, any defined microbiological media of the present invention may be used in this method. Defined microbiological media are described, for example, in International Patent Application WO 2007/061848. Each defined microbiological media represents a separate embodiment of the present invention.

In another embodiment, the present invention provides a method of eliciting an anti-Her-2 immune response in a subject, comprising administering to the subject a recombinant peptide of the present invention, thereby eliciting an anti-Her-2 immune response in a subject.

In another embodiment, the present invention provides a method of eliciting an anti-Her-2 immune response in a subject, comprising administering to the subject a nucleotide molecule of the present invention, thereby eliciting an anti-Her-2 immune response in a subject.

In another embodiment, the present invention provides a method of treating a Her-2-expressing tumor in a subject, comprising administering to the subject a recombinant peptide of the present invention, thereby treating a Her-2-expressing tumor in a subject.

In another embodiment, the present invention provides a method of treating a Her-2-expressing tumor in a subject, comprising administering to the subject a nucleotide molecule of the present invention, thereby treating a Her-2-expressing tumor in a subject.

In another embodiment, the present invention provides a method of reducing an incidence of a Her-2-expressing tumor in a subject, comprising administering to the subject a recombinant peptide of the present invention, thereby reducing an incidence of a Her-2-expressing tumor in a subject.

In another embodiment, the present invention provides a method of reducing an incidence of a Her-2-expressing tumor in a subject, comprising administering to the subject a nucleotide molecule of the present invention, thereby reducing an incidence of a Her-2-expressing tumor in a subject.

In another embodiment, a method of the present invention comprises administration of a vaccine comprising a recombinant peptide of the present invention. In another embodiment, a method of the present invention comprises administration of an immunogenic composition comprising a recombinant peptide of the present invention. In another embodiment, a method of the present invention comprises administration of a recombinant vaccine vector comprising a recombinant peptide of the present invention. In another embodiment, a method of the present invention comprises administration of a recombinant Listeria strain comprising a recombinant peptide of the present invention. In another embodiment, a method of the present invention comprises administration of a vaccine comprising a nucleotide molecule of the present invention. In another embodiment, a method of the present invention comprises administration of an immunogenic composition comprising a nucleotide molecule of the present invention. In another embodiment, a method of the present invention comprises administration of a recombinant vaccine vector comprising a nucleotide molecule of the present invention. In another embodiment, a method of the present invention comprises administration of a recombinant Listeria strain comprising a recombinant nucleotide molecule of the present invention. Each possibility represents a separate embodiment of the present invention.

In another embodiment, a method of the present invention further comprises administration of a chemotherapy agent targeting the protein against which the vaccine is directed. In another embodiment, the chemotherapy agent inhibits an activity of the protein. In another embodiment, the chemotherapy agent and a vaccine of the present invention exhibit synergy. In another embodiment, the chemotherapy agent and the vaccine exhibit an additive effect. Each possibility represents a separate embodiment of the present invention.

In one embodiment, the cancer treated by a method of the present invention is breast cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma. In another embodiment the cancer is a brain tumor. Each possibility represents a separate embodiment of the present invention.

In another embodiment, the present invention provides a method of testing a vaccine against an oncogenic protein, the method comprising the steps of immunizing a subject against the oncogenic protein and analyzing sequences of the gene encoding the oncogenic protein in tumor cells for mutations following vaccination. In another embodiment, appearance of escape mutations indicates that the vaccine is efficacious. In another embodiment, appearance of escape mutations indicates that the vaccine exerts immune pressure on tumors expressing the target protein. In another embodiment, this method is utilized to design the vaccine. In another embodiment, the subject is a human. In another embodiment, the subject is an animal. In another embodiment, the subject is an animal model for a cancer or tumor known to express the oncogenic protein. In another embodiment, the step of vaccination is in vivo. In another embodiment, the step of vaccination is in vitro. In another embodiment, the step of vaccination is ex vivo. As provided herein, information can be obtained using methods of the present invention that is useful in the selection from alternate antigenic delivery vehicles. In another embodiment, the information obtained is useful in determining the optimum order of administration of the delivery vehicles. In another embodiment, the information obtained is useful in determining the location of CTL epitopes in a tumor-associated protein (Example 5). In another embodiment, the information obtained is useful in determining the optimum dosage of the delivery vehicles. In another embodiment, the information obtained is useful in determining the pattern of administration of the delivery vehicles (e.g. whether they are administered together or sequentially. In another embodiment, the alternate antigenic delivery vehicles are different fragments of an oncogenic vehicle.

In another embodiment of methods and compositions of the present invention, sequential administration of several fragments of a tumor-associated protein enables a larger dosage of each fragment than would be possible in mixtures of the fragments were administered. In another embodiment, sequential administration enables a larger dosage of each fragment than would be considered safe in mixtures were administered. In another embodiment, sequential administration achieves an effective concentration of each particular fragment, enabling an immune response of sufficient strength to achieve the desired clinical endpoint. As provided herein, vaccines doses of a particular construct after the initial dose for that construct are, under the conditions utilized herein, increasingly less effective due to the development of site specific mutations. Since the initial exposure is the most important exposure, maximizing the dosage of the initial vaccination, by sequential administration of different constructs, increases vaccine efficacy. In another embodiment, such a regimen maximizes the therapeutic potential of each individual fragment and the combination overall. Each possibility represents a separate embodiment of the present invention.

In another embodiment, the present invention provides a method of inducing a regression of a tumor expressing a tumor-associated protein, comprising performing a method of the present invention, thereby inducing a regression of a tumor expressing a tumor-associated protein.

In another embodiment, the present invention provides a method of overcoming an immune tolerance to a tumor expressing a tumor-associated protein, comprising performing a method of the present invention, thereby overcoming an immune tolerance to a tumor expressing a tumor-associated protein.

In another embodiment, the present invention provides a method of reducing the incidence of relapse of a tumor expressing a tumor-associated protein, comprising performing a method of the present invention, thereby reducing the incidence of relapse of a tumor expressing a tumor-associated protein.

In another embodiment, the present invention provides a method of protecting a human subject against a tumor expressing a tumor-associated protein, the method comprising the step of administering to the human subject a composition comprising a recombinant Listeria strain of the present invention, whereby the subject mounts an immune response against the tumor, thereby protecting a human subject against a tumor expressing a tumor-associated protein.

The protein of interest of methods and compositions of the present invention is, in another embodiment, a whole protein. In another embodiment, the protein of interest is a protein fragment. In another embodiment, “protein” refers to an antigen. Each possibility represents a separate embodiment of the present invention.

Various embodiments of dosage ranges of vaccines of the present invention can be used in methods of the present invention. In one embodiment, the dosage of peptides or protein fragments administered is in the range of 1-80 microgram (mcg)/day. In another embodiment, the dosage is 5-80 mcg/day. In another embodiment the dosage is 20-80 mcg/day. In another embodiment the dosage is 20-60 mcg/day. In another embodiment the dosage is 40-60 mcg/day. In another embodiment the dosage is in a range of 45-60 mcg/day. In another embodiment the dosage is 15-25 mcg/day. In one embodiment, the dosage is 20 mcg/day. In another embodiment, the dosage is 40 mcg/day. In another embodiment, the dosage is 60 mcg/day. In another embodiment, the dosage is 80 mcg/day.

In another embodiment, the dosage is 10 mcg/dose. In another embodiment, the dosage is 20 mcg/dose. In another embodiment, the dosage is 30 mcg/dose. In another embodiment, the dosage is 40 mcg/dose. In another embodiment, the dosage is 60 mcg/dose. In another embodiment, the dosage is 80 mcg/dose. In another embodiment, the dosage is 100 mcg/dose. In another embodiment, the dosage is 150 mcg/dose. In another embodiment, the dosage is 200 mcg/dose. In another embodiment, the dosage is 300 mcg/dose. In another embodiment, the dosage is 400 mcg/dose. In another embodiment, the dosage is 600 mcg/dose. In another embodiment, the dosage is 800 mcg/dose. In another embodiment, the dosage is 1000 mcg/dose. In another embodiment, the dosage is 1500 mcg/dose. In another embodiment, the dosage is 2000 mcg/dose.

In another embodiment, the dosage is 10-20 mcg/dose. In another embodiment, the dosage is 20-30 mcg/dose. In another embodiment, the dosage is 20-40 mcg/dose. In another embodiment, the dosage is 30-60 mcg/dose. In another embodiment, the dosage is 40-80 mcg/dose. In another embodiment, the dosage is 50-100 mcg/dose. In another embodiment, the dosage is 50-150 mcg/dose. In another embodiment, the dosage is 100-200 mcg/dose. In another embodiment, the dosage is 200-300 mcg/dose. In another embodiment, the dosage is 300-400 mcg/dose. In another embodiment, the dosage is 400-600 mcg/dose. In another embodiment, the dosage is 500-800 mcg/dose. In another embodiment, the dosage is 800-1000 mcg/dose. In another embodiment, the dosage is 1000-1500 mcg/dose. In another embodiment, the dosage is 1500-2000 mcg/dose.

In another embodiment, the dosage is of recombinant vector administered is 0.0001 LD₅₀/dose. In another embodiment, the dosage is 0.00015 LD₅₀/dose. In another embodiment, the dosage is 0.0002 LD₅₀/dose. In another embodiment, the dosage is 0.0003 LD₅₀/dose. In another embodiment, the dosage is 0.0005 LD₅₀/dose. In another embodiment, the dosage is 0.0007 LD₅₀/dose. In another embodiment, the dosage is 0.001 LD₅₀/dose. In another embodiment, the dosage is 0.0015 LD₅₀/dose. In another embodiment, the dosage is 0.002 LD₅₀/dose. In another embodiment, the dosage is 0.003 LD₅₀/dose. In another embodiment, the dosage is 0.005 LD₅₀/dose. In another embodiment, the dosage is 0.007 LD₅₀/dose. In another embodiment, the dosage is 0.01 LD₅₀/dose. In another embodiment, the dosage is 0.015 LD₅₀/dose. In another embodiment, the dosage is 0.02 LD₅₀/dose. In another embodiment, the dosage is 0.03 LD₅₀/dose. In another embodiment, the dosage is 0.05 LD₅₀/dose. In another embodiment, the dosage is 0.07 LD₅₀/dose. In another embodiment, the dosage is 0.1 LD₅₀/dose. In another embodiment, the dosage is 0.15 LD₅₀/dose. In another embodiment, the dosage is 0.2 LD₅₀/dose. In another embodiment, the dosage is 0.3 LD₅₀/dose. In another embodiment, the dosage is 0.5 LD₅₀/dose.

In another embodiment, a recombinant peptide of the present invention is homologous to a Her-2 fragment, LLO protein or fragment thereof, ActA protein or fragment thereof, or PEST-like sequence disclosed herein. The terms “homology,” “homologous,” etc, when in reference to any protein or peptide, refer, in another embodiment, to a percentage of amino acid (AA) residues that are identical with the comparison sequence, after aligning the sequences and introducing gaps, if necessary, to achieve the maximum percent homology, and not considering any conservative substitutions as part of the sequence identity. Methods and computer programs for the alignment are well known in the art. In another embodiment, a peptide of the present invention is homologous to a peptide enumerated herein.

Homology is, in another embodiment, determined by computer algorithm for sequence alignment, by methods well described in the art. For example, computer algorithm analysis of nucleic acid sequence homology can include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a Her-2 sequence selected from SEQ ID No: 11-17, 32-33, and 35-60 of greater than 60%. In another embodiment, “homology” refers to identity to 1 of SEQ ID No: 11-17, 32-33, and 35-60 of greater than 65%. In another embodiment, “homology” refers to identity to 1 of SEQ ID No: 11-17, 32-33, and 35-60 of greater than 67%. In another embodiment, “homology” refers to identity to 1 of SEQ ID No: 11-17, 32-33, and 35-60 of greater than 70%. In another embodiment, “homology” refers to identity to a sequence selected from SEQ ID No: 11-17, 32-33, and 35-60 of greater than 72%. In another embodiment, “homology” refers to identity to 1 of SEQ ID No: 11-17, 32-33, and 35-60 of greater than 75%. In another embodiment, “homology” refers to identity to a sequence selected from SEQ ID No: 11-17, 32-33, and 35-60 of greater than 78%. In another embodiment, “homology” refers to identity to 1 of SEQ ID No: 11-17, 32-33, and 35-60 of greater than 80%. In another embodiment, “homology” refers to identity to 1 of SEQ ID No: 11-17, 32-33, and 35-60 of greater than 82%. In another embodiment, “homology” refers to identity to a sequence selected from SEQ ID No: 11-17, 32-33, and 35-60 of greater than 83%. In another embodiment, “homology” refers to identity to 1 of SEQ ID No: 11-17, 32-33, and 35-60 of greater than 85%. In another embodiment, “homology” refers to identity to 1 of SEQ ID No: 11-17, 32-33, and 35-60 of greater than 87%. In another embodiment, “homology” refers to identity to a sequence selected from SEQ ID No: 11-17, 32-33, and 35-60 of greater than 88%. In another embodiment, “homology” refers to identity to 1 of SEQ ID No: 11-17, 32-33, and 35-60 of greater than 90%. In another embodiment, “homology” refers to identity to 1 of SEQ ID No: 11-17, 32-33, and 35-60 of greater than 92%. In another embodiment, “homology” refers to identity to a sequence selected from SEQ ID No: 11-17, 32-33, and 35-60 of greater than 93%. In another embodiment, “homology” refers to identity to 1 of SEQ ID No: 11-17, 32-33, and 35-60 of greater than 95%. In another embodiment, “homology” refers to identity to a sequence selected from SEQ ID No: 11-17, 32-33, and 35-60 of greater than 96%. In another embodiment, “homology” refers to identity to 1 of SEQ ID No: 11-17, 32-33, and 35-60 of greater than 97%. In another embodiment, “homology” refers to identity to 1 of SEQ ID No: 11-17, 32-33, and 35-60 of greater than 98%. In another embodiment, “homology” refers to identity to 1 of SEQ ID No: 11-17, 32-33, and 35-60 of greater than 99%. In another embodiment, “homology” refers to identity to 1 of SEQ ID No: 11-17, 32-33, and 35-60 of 100%. Each possibility represents a separate embodiment of the present invention.

In another embodiment, “homology” refers to identity to an LLO sequence selected from SEQ ID No: 18, 19, and 34 of greater than 60%. In another embodiment, “homology” refers to identity to 1 of SEQ ID No: 18, 19, and 34 of greater than 65%. In another embodiment, “homology” refers to identity to 1 of SEQ ID No: 18, 19, and 34 of greater than 67%. In another embodiment, “homology” refers to identity to 1 of SEQ ID No: 18, 19, and 34 of greater than 70%. In another embodiment, “homology” refers to identity to a sequence selected from SEQ ID No: 18, 19, and 34 of greater than 72%. In another embodiment, “homology” refers to identity to 1 of SEQ ID No: 18, 19, and 34 of greater than 75%. In another embodiment, “homology” refers to identity to a sequence selected from SEQ ID No: 18, 19, and 34 of greater than 78%. In another embodiment, “homology” refers to identity to 1 of SEQ ID No: 18, 19, and 34 of greater than 80%. In another embodiment, “homology” refers to identity to 1 of SEQ ID No: 18, 19, and 34 of greater than 82%. In another embodiment, “homology” refers to identity to a sequence selected from SEQ ID No: 18, 19, and 34 of greater than 83%. In another embodiment, “homology” refers to identity to 1 of SEQ ID No: 18, 19, and 34 of greater than 85%. In another embodiment, “homology” refers to identity to 1 of SEQ ID No: 18, 19, and 34 of greater than 87%. In another embodiment, “homology” refers to identity to a sequence selected from SEQ ID No: 18, 19, and 34 of greater than 88%. In another embodiment, “homology” refers to identity to 1 of SEQ ID No: 18, 19, and 34 of greater than 90%. In another embodiment, “homology” refers to identity to 1 of SEQ ID No: 18, 19, and 34 of greater than 92%. In another embodiment, “homology” refers to identity to a sequence selected from SEQ ID No: 18, 19, and 34 of greater than 93%. In another embodiment, “homology” refers to identity to 1 of SEQ ID No: 18, 19, and 34 of greater than 95%. In another embodiment, “homology” refers to identity to a sequence selected from SEQ ID No: 18, 19, and 34 of greater than 96%. In another embodiment, “homology” refers to identity to 1 of SEQ ID No: 18, 19, and 34 of greater than 97%. In another embodiment, “homology” refers to identity to 1 of SEQ ID No: 18, 19, and 34 of greater than 98%. In another embodiment, “homology” refers to identity to 1 of SEQ ID No: 18, 19, and 34 of greater than 99%. In another embodiment, “homology” refers to identity to 1 of SEQ ID No: 18, 19, and 34 of 100%. Each possibility represents a separate embodiment of the present invention.

In another embodiment, “homology” refers to identity to an ActA sequence selected from SEQ ID No: 20-21 of greater than 60%. In another embodiment, “homology” refers to identity to 1 of SEQ ID No: 20-21 of greater than 65%. In another embodiment, “homology” refers to identity to 1 of SEQ ID No: 20-21 of greater than 67%. In another embodiment, “homology” refers to identity to 1 of SEQ ID No: 20-21 of greater than 70%. In another embodiment, “homology” refers to identity to a sequence selected from SEQ ID No: 20-21 of greater than 72%. In another embodiment, “homology” refers to identity to 1 of SEQ ID No: 20-21 of greater than 75%. In another embodiment, “homology” refers to identity to a sequence selected from SEQ ID No: 20-21 of greater than 78%. In another embodiment, “homology” refers to identity to 1 of SEQ ID No: 20-21 of greater than 80%. In another embodiment, “homology” refers to identity to 1 of SEQ ID No: 20-21 of greater than 82%. In another embodiment, “homology” refers to identity to a sequence selected from SEQ ID No: 20-21 of greater than 83%. In another embodiment, “homology” refers to identity to 1 of SEQ ID No: 20-21 of greater than 85%. In another embodiment, “homology” refers to identity to 1 of SEQ ID No: 20-21 of greater than 87%. In another embodiment, “homology” refers to identity to a sequence selected from SEQ ID No: 20-21 of greater than 88%. In another embodiment, “homology” refers to identity to 1 of SEQ ID No: 20-21 of greater than 90%. In another embodiment, “homology” refers to identity to 1 of SEQ ID No: 20-21 of greater than 92%. In another embodiment, “homology” refers to identity to a sequence selected from SEQ ID No: 20-21 of greater than 93%. In another embodiment, “homology” refers to identity to 1 of SEQ ID No: 20-21 of greater than 95%. In another embodiment, “homology” refers to identity to a sequence selected from SEQ ID No: 20-21 of greater than 96%. In another embodiment, “homology” refers to identity to 1 of SEQ ID No: 20-21 of greater than 97%. In another embodiment, “homology” refers to identity to 1 of SEQ ID No: 20-21 of greater than 98%. In another embodiment, “homology” refers to identity to 1 of SEQ ID No: 20-21 of greater than 99%. In another embodiment, “homology” refers to identity to 1 of SEQ ID No: 20-21 of 100%. Each possibility represents a separate embodiment of the present invention.

In another embodiment, “homology” refers to identity to a PEST-like sequence selected from SEQ ID No: 22-23 and 25-31 of greater than 60%. In another embodiment, “homology” refers to identity to 1 of SEQ ID No: 22-23 and 25-31 of greater than 65%. In another embodiment, “homology” refers to identity to 1 of SEQ ID No: 22-23 and 25-31 of greater than 67%. In another embodiment, “homology” refers to identity to 1 of SEQ ID No: 22-23 and 25-31 of greater than 70%. In another embodiment, “homology” refers to identity to a sequence selected from SEQ ID No: 22-23 and 25-31 of greater than 72%. In another embodiment, “homology” refers to identity to 1 of SEQ ID No: 22-23 and 25-31 of greater than 75%. In another embodiment, “homology” refers to identity to a sequence selected from SEQ ID No: 22-23 and 25-31 of greater than 78%. In another embodiment, “homology” refers to identity to 1 of SEQ ID No: 22-23 and 25-31 of greater than 80%. In another embodiment, “homology” refers to identity to 1 of SEQ ID No: 22-23 and 25-31 of greater than 82%. In another embodiment, “homology” refers to identity to a sequence selected from SEQ ID No: 22-23 and 25-31 of greater than 83%. In another embodiment, “homology” refers to identity to 1 of SEQ ID No: 22-23 and 25-31 of greater than 85%. In another embodiment, “homology” refers to identity to 1 of SEQ ID No: 22-23 and 25-31 of greater than 87%. In another embodiment, “homology” refers to identity to a sequence selected from SEQ ID No: 22-23 and 25-31 of greater than 88%. In another embodiment, “homology” refers to identity to 1 of SEQ ID No: 22-23 and 25-31 of greater than 90%. In another embodiment, “homology” refers to identity to 1 of SEQ ID No: 22-23 and 25-31 of greater than 92%. In another embodiment, “homology” refers to identity to a sequence selected from SEQ ID No: 22-23 and 25-31 of greater than 93%. In another embodiment, “homology” refers to identity to 1 of SEQ ID No: 22-23 and 25-31 of greater than 95%. In another embodiment, “homology” refers to identity to a sequence selected from SEQ ID No: 22-23 and 25-31 of greater than 96%. In another embodiment, “homology” refers to identity to 1 of SEQ ID No: 22-23 and 25-31 of greater than 97%. In another embodiment, “homology” refers to identity to 1 of SEQ ID No: 22-23 and 25-31 of greater than 98%. In another embodiment, “homology” refers to identity to 1 of SEQ ID No: 22-23 and 25-31 of greater than 99%. In another embodiment, “homology” refers to identity to 1 of SEQ ID No: 22-23 and 25-31 of 100%. Each possibility represents a separate embodiment of the present invention.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y.). In other embodiments, methods of hybridization are carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

In another embodiment of the present invention, “nucleic acids” or “nucleotide” refers to a string of at least two base-sugar-phosphate combinations. The term includes, in one embodiment, DNA and RNA. “Nucleotides” refers, in one embodiment, to the monomeric units of nucleic acid polymers. RNA is, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA can be, in other embodiments, in form of plasmid DNA, viral DNA, linear DNA, or chromosomal DNA or derivatives of these groups. In addition, these forms of DNA and RNA can be single, double, triple, or quadruple stranded. The term also includes, in another embodiment, artificial nucleic acids that contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothioate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention.

Protein and/or peptide homology for any amino acid sequence listed herein is determined, in another embodiment, by methods well described in the art, including immunoblot analysis, or via computer algorithm analysis of amino acid sequences, utilizing any of a number of software packages available, via established methods. Some of these packages include the FASTA, BLAST, MPsrch or Scanps packages, and, in another embodiment, employ the use of the Smith and Waterman algorithms, and/or global/local or BLOCKS alignments for analysis, for example. Each method of determining homology represents a separate embodiment of the present invention.

In another embodiment, the present invention provides a kit comprising a compound or composition utilized in performing a method of the present invention. In another embodiment, the present invention provides a kit comprising a composition, tool, or instrument of the present invention.

### Pharmaceutical Compositions and Methods of Administration

In one embodiment, the methods of the present invention comprise administering a pharmaceutical composition comprising a recombinant peptide and/or its analog, derivative, isomer, metabolite, pharmaceutically acceptable salt, pharmaceutical product, hydrate, N-oxide, or any combination thereof; and a pharmaceutically acceptable carrier.

The pharmaceutical compositions containing the peptide can be, in another embodiment, administered to a subject by any method known to a person skilled in the art, such as parenterally, transmucosally, transdermally, intramuscularly, intravenously, intra-dermally, subcutaneously, intra-peritonealy, intra-ventricularly, intra-cranially, intra-vaginally or intra-tumorally.

In another embodiment of methods and compositions of the present invention, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, the pharmaceutical compositions are administered by intravenous, intra-arterial, or intramuscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, the pharmaceutical compositions are administered topically to body surfaces and are thus formulated in a form suitable for topical administration. Suitable topical formulations include gels, ointments, creams, lotions, drops and the like. For topical administration, the peptides are prepared and applied as solutions, suspensions, or emulsions in a physiologically acceptable diluent with or without a pharmaceutical carrier.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of peptide over a period of time.

In another embodiment, the active compound is delivered in a vesicle, e.g. a liposome.

In other embodiments, carriers or diluents used in methods of the present invention include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In another embodiment, parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In other embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hydroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants. Each of the above excipients represents a separate embodiment of the present invention.

Each of the above additives, excipients, formulations and methods of administration represents a separate embodiment of the present invention.

### EXPERIMENTAL DETAILS SECTION

### Example 1

### Generation of L. monocytogenes Strains that Secrete LLO Fragments Fused to Her-2 Fragments

### Materials and Experimental Methods

### Subcloning

pGG-55, the backbone of the Listeria Her-2 constructs used in the Examples, was created from pAM401. pAM401, a shuttle vector able to replicate in both gram⁺ and gram⁻ bacteria, contains a gram⁺ chloramphenicol resistance gene and a gram⁻ tetracycline resistance gene (Wirth, R et al, J Bacteriol, 165: 831, 1986). To produce pGG-55, an hly-HPV 16 E7 fusion gene (including the hly promoter and the portion of hly encoding the first 441 amino acid (AA) of LLO; referred to below as “ΔLLO”) and the pluripotent transcription factor, prfA (positive regulatory factor of listeriolysin expression) gene were cloned into pAM401 (FIG. 1).

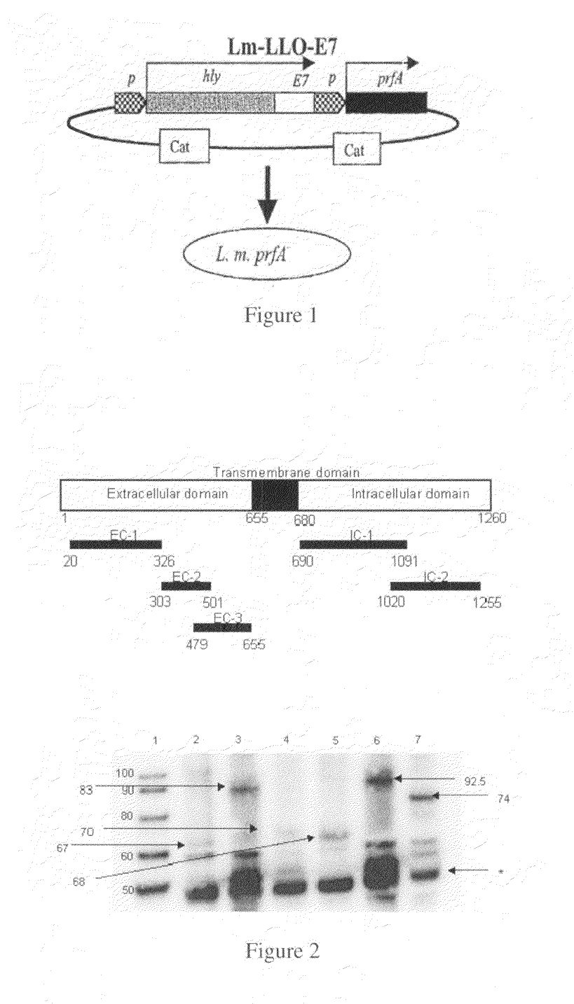

L. monocytogenes (LM) strains Lm-ΔLLO-EC1, Lm-ΔLLO-EC2, Lm-ΔLLO-EC3, Lm-ΔLLO-IC1, and Lm-ΔLLO-IC2 each contain a plasmid expressing a fragment of rat Her-2 fused to the Listerial hly gene. The following overlapping fragments of the extracellular and intracellular domains of Her-2 were cloned into the plasmid pGG-55: base pairs (bp) 74-994, (Lm-ΔLLO-EC1; corresponding to AA 20-326 of Her-2), 923-1519 (Lm-ΔLLO-EC2; corresponding to AA 303-501), 1451-1981 (Lm-ΔLLO-EC3; corresponding to AA 479-655), 2084-3259 (Lm-ΔLLO-IC1; corresponding to AA 690-1081), and 3073-3796 (Lm-ΔLLO-IC2; corresponding to AA 1020-1255). The fragments are depicted in FIG. 2A. The LD₅₀ of EC1, EC2, EC3, IC1, and IC2 were 1×10⁸, 1×10⁹, 5×10⁸, 1×10⁸ and 1×10⁸, respectively.

Each Her-2 fragment was amplified by PCR from the pNINA plasmid, which contains the full-length rat Her-2 gene, using the following primers. Restriction sites (XhoI in the case of EC1, IC1, and IC2 5′ primers; SpeI for the 3′ primers; and SalI for the EC2 and EC3 5′ primers) are underlined, and the FLAG tag sequence in the EC2 and EC3 the 3′ primers are indicated by italics:

(SEQ ID No: 1)

ECI: 5′ primer:
CACGCGGATGAAATCGATAAGCTCGAGCCCCCCGGAATCGCGGGCAC;

(SEQ ID No: 2)

3′ primer:
CCGGACTAGTGACCTCTTGGTTATTCGGGGGACACACC.

(SEQ ID No: 3)

EC2: 5′ primer:
CCGGGTCGACTGCCCCTACAACTACCTGTCTACG;

(SEQ ID No: 4)

3′ primer:
CCGGACTAGTTTACTTGTCATCGTCGTCCTTGTAGTC CCCACTGTGGAGC
AGGGCCTG;

(SEQ ID No: 5)

EC3: 5′ primer:
CCGGGTCGACTGCTTTGTACACACTGTACCTTGG;

(SEQ ID No: 6)

3′ primer:
CCGGACTAGTTTACTTGTCATCGTCGTCCTTGTAGTCC GGGCTGGCTCTC
TGCTCTGC;

(SEQ ID No: 7)

IC1: 5′ primer:
CCGGCTCGAGTATACGATGCGTAGGCTGCTGCAGG;

(SEQ ID No: 8)

3′ primer:
CCGGACTAGTAGCCAGTGGAGATCTGGGGGGCCC;

(SEQ ID No: 9)

IC2: 5′ primer:
CCGGCTCGAGGGTGACCTGGTAGACGCTGAAG
and

(SEQ ID No: 10)

3′ primer:
CCGGACTAGTTACAGGTACATCCAGGCCTAGG.

Fragments were amplified by PCR and cloned into the pCR 2.1 expression system (Invitrogen, Carlsbad, Calif.), then excised with the delineated enzymes. The E7 gene was excised from the pGG-55 plasmid using Xho I and Spe I, then the Her-2 fragment was fusion was ligated into the E7 site (ends digested with Sal I are compatible with XhoI ends). XFL-7, a prfA negative strain of LM, (Gunn G R et al, J Immunol 167: 647, 2001) was transfected with the plasmids by electroporation.

### Bacteria

Bacteria were grown in brain heart infusion medium (BD, Sparks, Md.) with 50 μg/ml chloramphenicol and were frozen in 1 ml aliquots at −80° C.

### Western Blots

ΔLLO-Her-2 expressing strains were grown overnight at 37° C. in Luria-Bertani (LB) medium with 50 microgram per milliliter (μg/ml) chloramphenicol. Supernatants were TCA precipitated and resuspended in 1× LDS sample buffer (Invitrogen, San Diego, Calif.). 15 microliter (μl) of each sample was loaded on a 4-12% Bis-Tris SDS-PAGE gel (Invitrogen, San Diego, Calif.). Gels were transferred to a Immobilon-P polyvinylidene fluoride membrane (Millipore, Billerica, Mass.) and blotted with a polyclonal rabbit serum recognizing residues 1-30 of LLO, followed by HRP-conjugated anti-rabbit antibody (Amersham Pharmacia Biotech, UK).

### Statistical Analyses

Statistical analyses were performed using Student's t-test throughout the Examples.

### Results

Five recombinant LM strains were constructed that express and secrete overlapping fragments of the rat Her-2 gene fused to the N-terminal portion of L. monocytogenes LLO protein (FIG. 2A). The signal sequence and transmembrane domain of Her-2 were not included among the fragments due to their hydrophobicity and the inability of LM to secrete extremely hydrophobic domains. Secretion of each Her-2 fragment was confirmed by Western blot (FIG. 2B). Molecular weights of the proteins LM-ΔLLO-EC1, Lm-ΔLLO-EC2, Lm-ΔLLO-EC3, Lm-ΔLLO-IC1, and Lm-ΔLLO-IC2 were 83, 70, 68, 92.5, and 74-kDa (kilodalton), respectively. The strains were attenuated relative to the wild-type 10403S strain, exhibiting virulences comparable to Lm-ΔLLO-E7; namely having an LD₅₀ of 1×10⁸, 5×10⁸, 1×10⁹, 1×10⁸, and 1×10⁸ colony forming units (CFU), respectively.

### Example 2

### Delay in Tumor Growth Following Protein Tyrosine Kinase Vaccination

### Materials and Experimental Methods

### Examples 2-5

### Mice

FVB/N HER-2/neu transgenic mice were housed and bred at the Veterans' Administration Hospital affiliated with the University of Pennsylvania. All experiments were in accordance with regulations by the Institutional Animal Care and Use Committee (IACUC) of the University of Pennsylvania and the Veterans' Administration.

### Listeria Vaccine Strains

LM-NY-ESO-1, constructed similarly to the Her-2 vaccines, was provided by Dr. Paulo Maciag, University of Pennsylvania. Bacteria were grown in brain heart infusion medium (BD, Sparks, Md.) with 50 micrograms (mcg)/milliliter (ml) chloramphenicol and frozen in 1 ml aliquots at −80° C. Vaccines were thawed and washed twice with sterile PBS, then resuspended in PBS, prior to injections.

### Peptides

Peptides were synthesized as custom peptides by EZBiolab, Inc. (Westfield, Ind.). The sequences were as follows: GSGAFGTVYK (AA 732-741); b. AFGTVYKGI (735-743); c. PYVSRLLGI (785-793); d. TSPKANKEI (764-772); e. RPRFRELVSE (971-980); f. STVQLVTQL (797-805); and g. KITDFGLARL (865-874).

### Spontaneous Tumor Protection Assay

Six-week old female FVB/N HER-2/neu transgenic mice (n=12 or 15) were vaccinated at 6, 9, 12, 15, and 18 weeks of age, with 0.1 LD₅₀ of one of the Lm-LLO constructs or Lm-LLO-NYESO-1, or mock-vaccinated with PBS. Mice were checked every 2 days by palpation of the mammary tissue for the onset of tumor growth.

### Analysis of Mutations and Mapping of the Location of the Mutations

Tumors that arose spontaneously in the tumor protection assay were harvested and DNA was extracted using a Puregene Genomic DNA Purification Kit (Gentra Systems, Minneapolis, Minn.). The entire gene was PCR amplified in triplicate and sequenced by the University of Pennsylvania Sequencing Facility. Sequences were analyzed using Sequencher 3.0 (Gene Codes Corp., Ann Arbor, Mich.). Mutations that were not detected in each of the triplicate PCR reaction sequences were discarded as PCR-induced mutations.

Mutation rate was calculated by dividing the number of mutations that occurred the total AA for each fragment (EC1: 307; EC2: 199; EC3: 177; IC1: 392; IC2: 240).

Kinase domain mutations were mapped onto an EGF kinase domain using PyMol (DeLano Scientific, LLC, San Francisco, Calif.).

### Cell Lines

The FVB/N syngeneic NT-2 tumor cell line was developed from a spontaneously occurring mammary tumor in an FVB/N HER-2/neu transgenic mouse. NT-2 tumor cells constitutively express low levels of rat HER-2/neu and are tumorigenic in wild type syngeneic mice. NT-2 cells were grown at 37° C. with 5% CO₂ in RPMI 1640 medium+20% FCS, 10.2 mM HEPES, 2 mM L-glutamine, 100 mcM nonessential AA, 1 mM sodium pyruvate, 50 U/ml penicillin G, 50 mcg/ml streptomycin, 20 mcg/ml insulin, and 2 mcg/ml gentamycin. NIH 3T3 cells, a mouse fibroblast line that expresses MHC class I molecules of the “q” haplotype, were cultured at 37° C. with 5% CO₂ in DMEM+10% FCS, 2 mM L-glutamine, 100 μM nonessential AA, 1 mM sodium pyruvate, 50 U/ml penicillin G, and 50 μg/ml streptomycin.

### CTL Assays

Six week old female HER-2/neu transgenic mice were vaccinated with 0.1 LD₅₀ of Lm-LLO-IC1 or 200 microliter (mcl) PBS. 9 days later spleens were harvested and splenocytes were cultured in vitro for 4 days with irradiated (20,000 rads) NT-2 tumor cells at a 10:1 splenocyte: tumor cell ratio with 20 U/ml IL-2. 4 days later splenocytes were harvested and used in a CTL assay with 3T3 target cells loaded with 1 μg/ml of the target peptides. Target cells were labeled with ⁵¹Cr and cultured with splenocytes at effector: target ratios of 200:1, 100:1, 50:1, and 25:1 in triplicate for 4 hours. Following the incubation, 100 mcl of supernatant was assayed for ⁵¹Cr release. Percent specific lysis was determined as [(experimental counts−spontaneous counts)/(total counts (stimulated by addition of 2% Triton X)−spontaneous counts)]×100.

### Statistical Analysis

Statistical analysis of spontaneous tumor growth utilized the Kaplan-Meier Log Rank test and was performed with the SPSS 11 statistical package (SPSS, Inc, Chicago, Ill.).

### Results

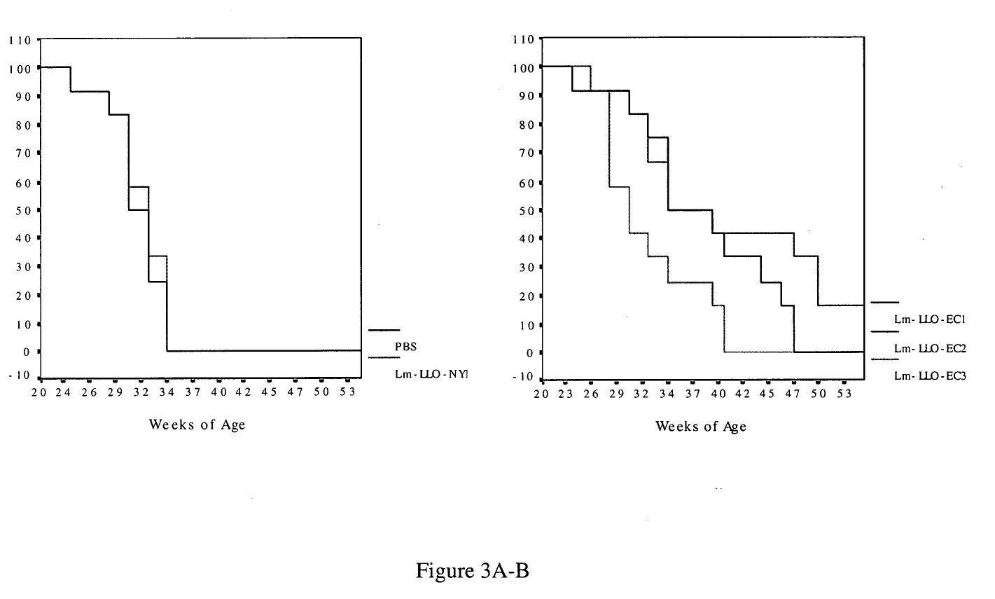

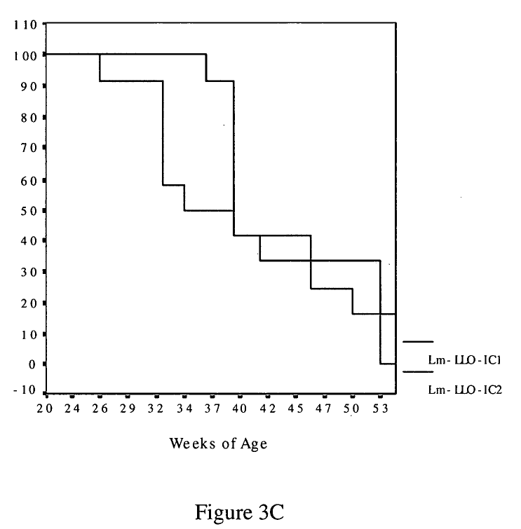

The HER-2/neu protein tyrosine kinase domain is fully contained within Lm-ΔLLO-IC1. This region of the rat neu gene is greater than 90% homologous to both the mouse and human neu genes, which was expected to result in more immune tolerance to this region, in addition to the tolerance conferred by the expression of the transgene. Thus, the Lm-ΔLLO-IC1 vaccine was not expected to effectively prevent development of spontaneous tumors in FVB/N HER-2/neu transgenic mice. However, the Lm-ΔLLO-IC1 vaccine delayed tumor growth onset in transgenic mice significantly more (p=0.00001) than the other 4 Lm-ΔLLO vaccines. In addition, the first mouse in the Lm-ΔLLO-IC1 group developed a tumor after all of the mock-vaccinated and Lm-ΔLLO-NYESO-1 (irrelevant antigen negative control) mice had already developed tumors (FIG. 3). The Lm-ΔLLO vaccines segregated into 3 efficacy groups for spontaneous tumor protection: Lm-ΔLLO-IC1 was the most efficacious; Lm-ΔLLO-EC3 (p=0.5685) was the least, and Lm-ΔLLO-EC1, Lm-ΔLLO-EC2, and Lm-ΔLLO-IC2 exhibited intermediate efficacy (p=0.0036, 0.0062, and 0.0054, respectively).

Thus, these vaccines delayed the onset of tumor growth by overcoming tolerance to HER-2/neu, in particular the protein kinase domain thereof. The anti-tumor efficacy of these vaccines was not permanent, as tumors did eventually grow in each of the vaccine groups.

### Example 3

### Specific Mutations in HER-2/neu Following LM-LLO-HER-2/neu Vaccination

To determine how whether the tumors had down regulated HER-2/neu expression to overcome the anti-HER-2/neu immune responses, expression of HER-2/neu was measured in the tumor cells that had grown despite vaccination. Expression of HER-2/neu was retained, indicating that another escape mechanism had been utilized. To determine if the oncogene was mutating in order to escape from immune pressure, HER-2/neu gene sequences from the tumors were analyzed. Spontaneous tumors that grew in the vaccinated mice were specifically mutated in the region contained in each vaccine (Table 1). Other regions exhibited a low level of random mutation that was significantly less than the specific mutations in the regions of HER-2/neu targeted by vaccination (Table 2), with an average difference of 10-fold. Thus, the tumors specifically mutated in order to escape immune pressure.

The HER-2/neu tyrosine kinase domain (AA 725-972) is highly homologous to other kinase domains, and was not expected to be immunogenic due to tolerance mechanisms. The kinase domain of HER-2/neu did mutate, however, in response to vaccination. By contrast, in the absence of Lm-ΔLLO-IC1 vaccination, random mutations did not accumulate (Table 1; specific mutations within the kinase domain are indicated in bold font). Thus, the tumors were limited in their ability to mutate in regions that are essential for the function of HER-2/neu, such as the kinase domain.

### Example 4

### Delayed Tumor Growth Due to Mutations in the Kinase Domain

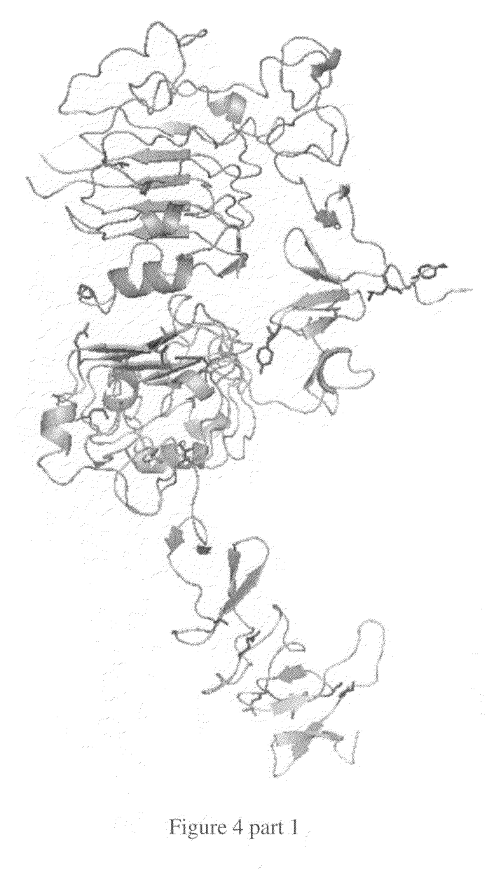

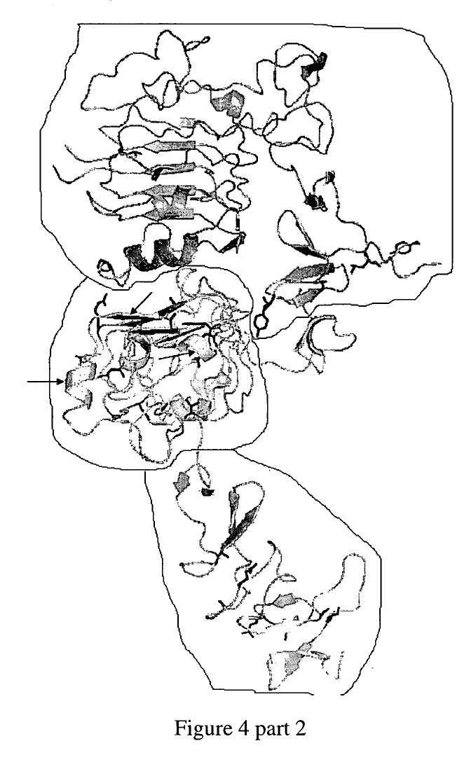

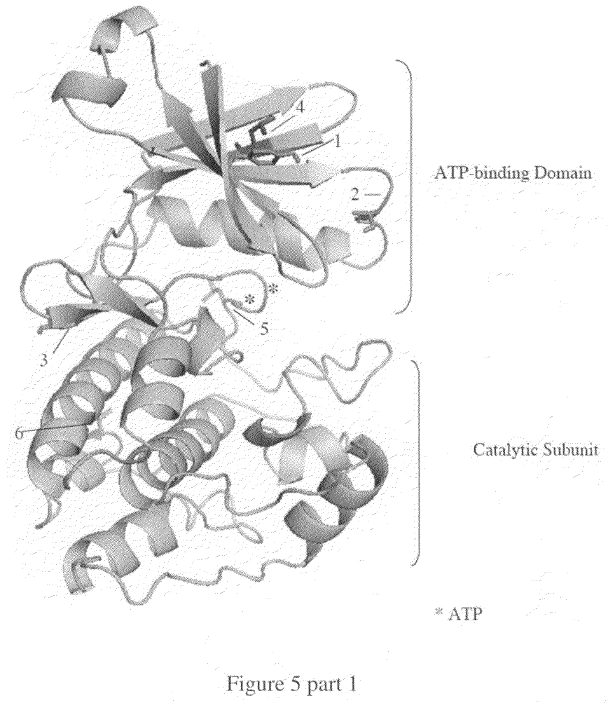

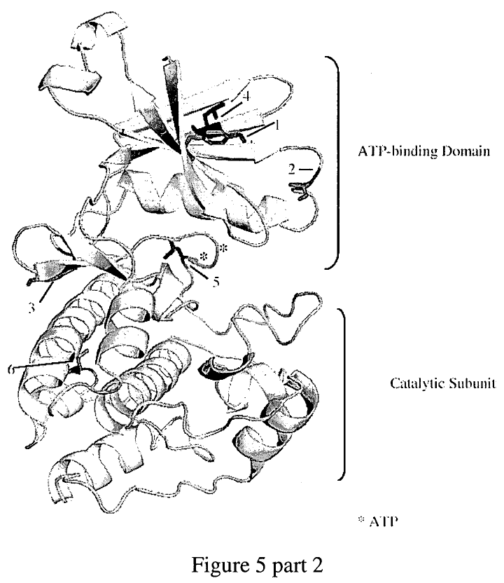

Mutations in the HER-2/neu extracellular domain were mapped onto a figure showing the 3-dimensional structure thereof (FIG. 4). Mutations in the tyrosine kinase domain were mapped onto a figure of the homologous epidermal growth factor receptor (EGFR) kinase domain as a model for the kinase domain (FIG. 5). The mutations occurred in regions unlikely to interfere with kinase domain function. There were no mutations within the catalytic subunit. The majority of the mutations also lay outside the ATP-binding domain. Mutation 2 (T764D) was in a loop region in the ATP-binding domain unlikely to have a great effect on the structure of the ATP-binding domain. The mutations most likely to alter the kinase domain function are 1 (Y740A) and 4 (deletion of L801), which are in β-sheets of the ATP-binding domain. As the tumors that grew out expressed HER-2/neu, these mutations do not abrogate kinase domain function but could alter ATP binding. Thus, under the conditions utilized herein, tumors targeted by Lm-ΔLLO-IC1 grow out after a delay due to a decrease in signaling and proliferative capacity.

### Example 5

### Kinase Domain Mutations all Lie within CD8⁺ T Cell Epitopes

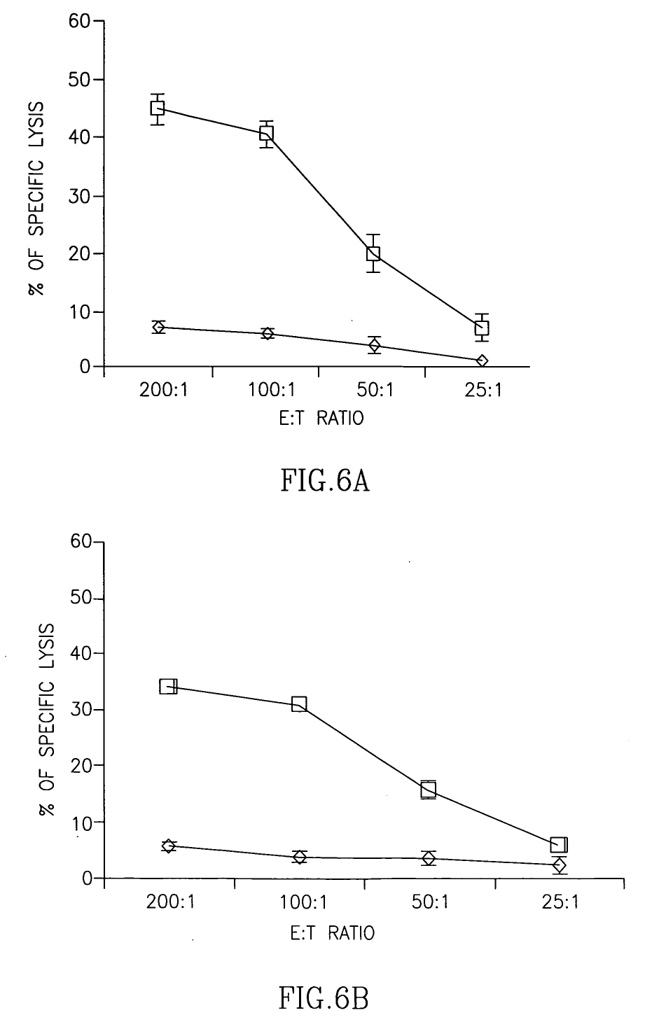

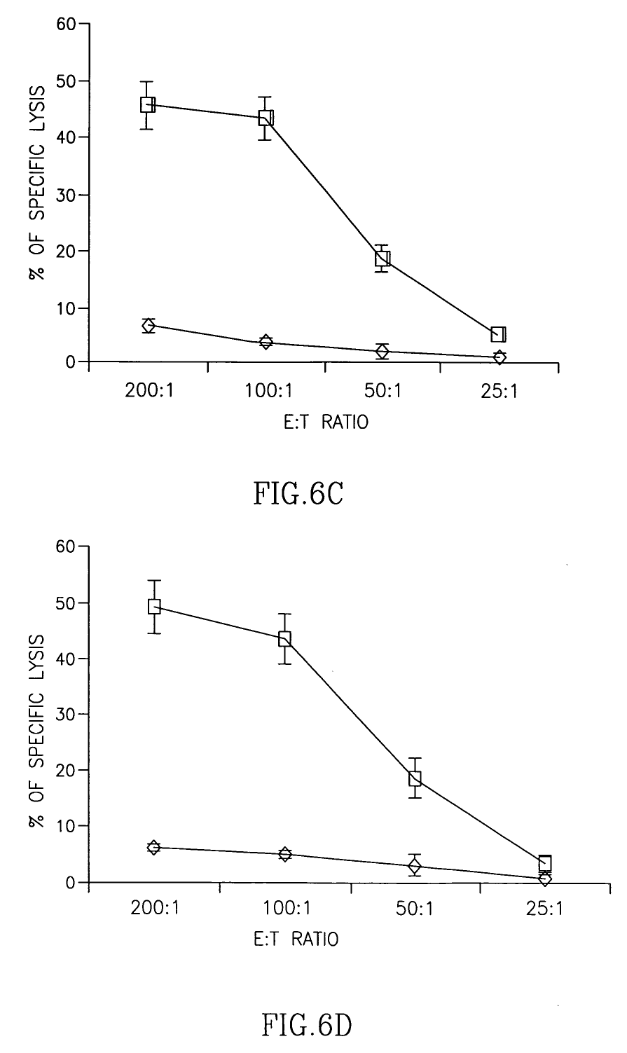

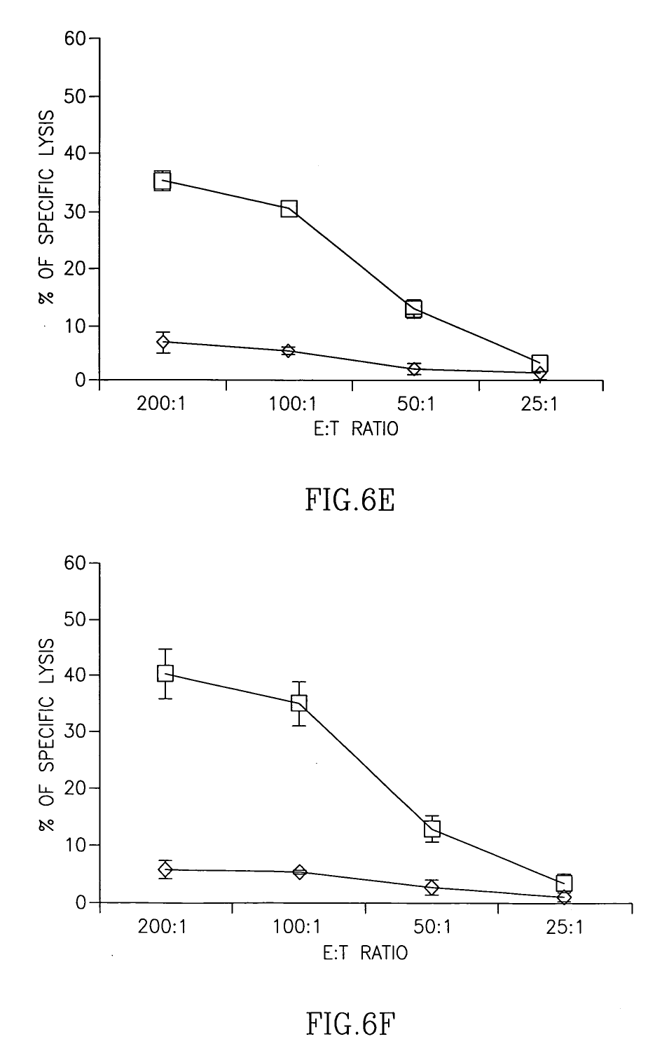

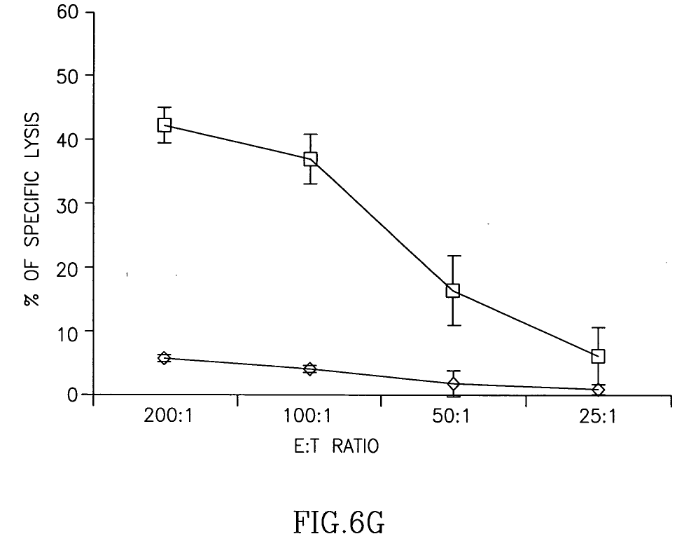

In FVB/N mice, rat HER-2/neu CD8⁺ T cell epitopes have not been identified within the intracellular domain or any part of the kinase domain. To determine whether mutation of specific residues in spontaneously arising tumors was due to CD8⁺ T cells recognition, the IC1 sequence was searched for putative epitopes containing the mutations, using the Rankpep predictor (Reche P A et al, Enhancement to the RANKPEP resource for the prediction of peptide binding to MHC molecules using profiles. Immunogenetics. 2004 September; 56(6):405-19). Target cells pulsed with peptides corresponding to the putative epitopes were recognized and killed by CD8⁺ T cells from Lm-ΔLLO-IC1 treated transgenic mice in a cytotoxic T lymphocyte (CTL) assay (FIG. 6A-C). Each mutation is contained within 1 or more novel CD8⁺ T cell epitopes, and could thus allow enable escape from detection by Lm-ΔLLO-IC1-elicited CD8⁺ T cells and growth in the face of an anti HER-2/neu immune response.

### Example 6

### Generation of LLO-HER-2 Vaccines Containing Fragments of Human HER-2 Protein

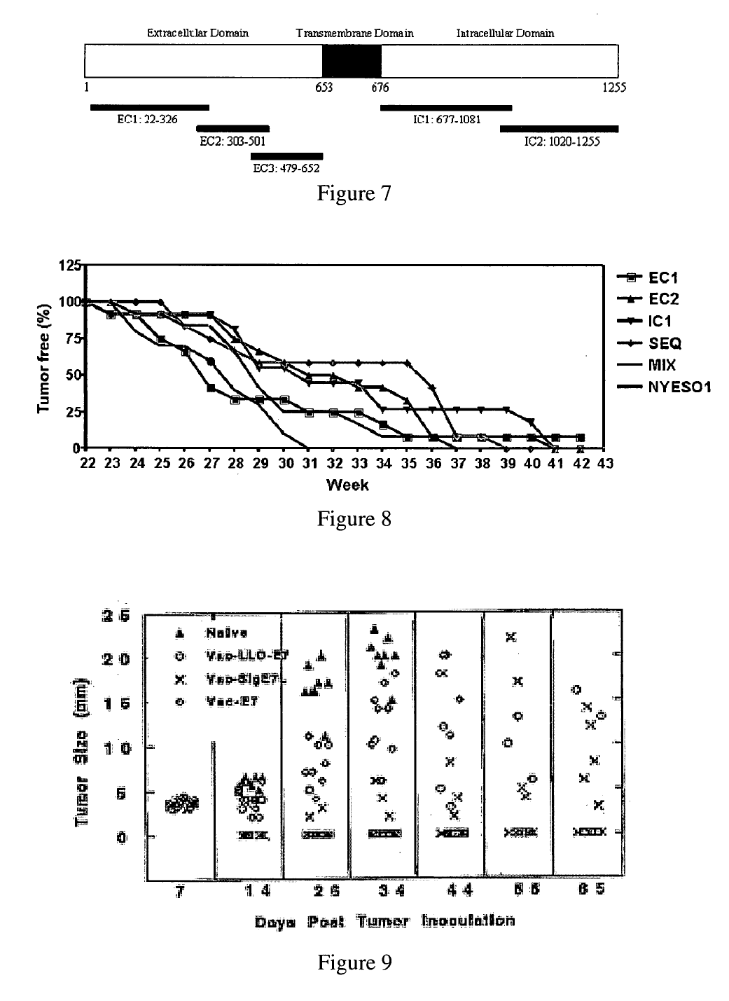

A similar strategy was used to express human Her-2/neu as was used for rat Her-2 (Example 1). The full-length HER-2 gene was split into five fragments, constituting overlapping fragments of the extracellular domain, (EC-1, EC-2 and EC-3) and the cytoplasmic domain (IC-1 and IC-2) (FIG. 7). Hydrophobic regions were not included in the constructs. These sequences differed from the rat sequences slightly due to the small dissimilarities between the two sequences. The human fragments corresponding to the rat fragments were 22-326, 303-501, 479-652, 677-1081, and 1020-1255.

### Example 7

### Sequential Administration of HER-2 Fragments Delays Tumor Growth

### Materials and Experimental Methods

FVB/HER-2 transgenic neu mice were inoculated intraperitoneally with 0.1 LD₅₀ total Listeria per inoculation. There were 12 mice each in the EC1, EC2, sequential, and mixed groups; 11 mice in the IC1 group, and 10 min in the NY-ESO-1 group. All mice were vaccinated at weeks 6, 9, 12, 15, 18, and 21. Mice received the same vaccine at each timepoint, with the exception of the sequential group, which was vaccinated with the Ic1 vaccine at weeks 6 and 9, the EC2 vaccine at weeks 12 and 15, and the EC1 vaccine at weeks 18 and 21. Mice exhibiting large tumors were removed and sacrificed.

### Results

To confirm that sequential administration of tumor fragments is superior to mixed or individual administration, FVB/HER-2 transgenic neu mice were administered Lm-LLO-HER-2/neu vaccines in various combinations and prevention of autochthonous breast tumor appearance was determined. Sequential administration of the tumor fragments showed superior ability to delay tumor formation compared to mixed or individual administration of the fragments (FIG. 8 and Table 4). Mice vaccinated with Lm-LLO-ESO-NY-1 (irrelevant antigen control) were not protected.

### Example 8

### Generation and Testing of Constructs Wherein the Target Epitope is Embedded within a Heterologous Peptide

### Materials and Experimental Methods

### Viruses

The influenza type A virus A/PR/8/34 belongs to the H1N1 subtype. The reassortment virus X31 (PR8×A/Aichi/68) differs from PR8 by expression of genes encoding H3 and N2, in place of H1N1, which are derived from the A/Aichi parent. Infectious virus stocks were grown in the allantoic cavity of 10 day old embryonated hen's eggs, and infectious allantoic fluid was stored in small aliquots at −70° C.

### Bacterial Strains and Growth Conditions

To construct plasmid pDP1659, the DNA fragment encoding the first 420 AA of LLO and its promoter and upstream regulatory sequences was PCR amplified and fused to the NP gene. Plasmid pDP2028 was constructed by cloning the prfA gene into the SalI site of pDP1659. Transformation of the prfA(−) strain DPL1075 with pDP2028 yielded strain DP-L2028, which secreted the fusion protein stably in vitro and in vivo.

Construction of strain DP-L2840. The splicing by overlap extension (SOE) PCR technique was used to replace the Kd restricted LLO epitope (residues 91-99) with the Kd restricted NP epitope, residues 147-155, and the modified hly gene was inserted into the PKSV7 temperature-sensitive vector to yield plasmid pDP2734. This plasmid was subsequently used to integrate the altered region into the bacterial chromosome.

Construction of strain DP-L2851. Plasmid pDP906 was derived by cloning a Sau96 fragment of the LM chromosome into pAM401. The chromosomal fragment codes for LLO and also includes the LLO promoter and the upstream regulatory sequences. No other complete open reading frames were present in this chromosomal fragment. Plasmid pDP906 was introduced into DP-L2840 by electroporation to yield DP-L2851. At every stage, engineering was verified by sequencing and restriction analysis.

### ⁵¹Cr Release Assays

Uninfected 5774 cells served as a negative control, and 5774 cells pulsed with the 147-158/R156⁻ NP peptide as a positive control. P815 cells were labeled, pulsed with NP epitope peptide or control peptide, and used as targets at a density of 10⁴ cells per well (round-bottom 96-well plates, Costar). Alternatively, P815 cells were infected with influenza virus as follows: 10⁶ cells were pelleted and resuspended in 100 mcL of serum-free medium. 100 mcL of infectious allantoic fluid containing 1000 hemagglutinating units (HAU) of A/PR/8 virus were added, and cells rocked gently at 37° C. for 1 h. Subsequently, medium containing serum was added and cells were incubated overnight at 32° C. under 5% CO₂. The next day, infected cells were labeled with ⁵¹Cr and used as targets. Released ⁵¹Cr was determined on 100 mcL of supernatant. Specific lysis was calculated as 100×[(X−S)/(T−S)], where X=experimental counts per minute (c.p.m.), S=spontaneous c.p.m., and T=total (1% Triton-induced) c.p.m. Data shown are representative of several experiments with similar results.

### Determination of Viral Titers in the Lungs of Immunized Mice

Mice were immunized i.v. with either 0.1-0.2 LD₅₀ of the LM strains, 10⁷ pfu of the vaccinia strains (provided by Dr Jack Bennink, Laboratory of Viral Diseases, NIAID) or with 100 mcl of infectious allantoic fluid of X31 virus. Three weeks later, mice were inoculated intranasally (i.n.) with 50 mcL influenza A/PR/8 virus in PBS. The amount of virus given corresponded to 0.25 LD₅₀. Intranasal administration was performed under metofane-induced anesthesia. Mice were sacrificed after 5 days, and their lungs were removed and homogenized in serum-free (0.1% BSA) Iscove's medium. Viral titers in tenfold dilutions of lung extracts were determined as described.

### Results

Several NP-expressing Lm strains, all described above, were created. In the case of Lm-ΔLLO-NP, NP was fused to ΔLLO in the same manner as other constructs described above. In the case of DP-L2840, the Kd restricted NP epitope, which spans AA 147-155 of NP33, was incorporated into (i.e. embedded within) the secreted LLO molecule. Since flanking sequences have been shown to influence the efficiency of epitope processing, the AA residues within the K^{d} restricted LLO epitope (91-99) were replaced with the residues from the K^{d} restricted epitope ensure correct processing. The resulting strain DP-L2840 did not possess hemolytic activity, as determined in vitro assays that measure lysis of sheep red blood cells, although it did secrete a mutant LLO molecule, as determined by Western blotting. The amount of LLO secreted by DP-L2840 was less than that precipitated from wild-type bacterial supernatants. To determine the effect of the difference in hemolytic activity, DP-L2840 was complemented in trans with a plasmid carrying a copy of the native hly gene, resulting in strain DP-L2851. DP-L2851 exhibited wild-type hemolytic activity on blood plates and grew more efficiently than DP-L2840 on a 5774 cell monolayer.

Cells infected with DP-L2028, but not DP-L2840, were able to present the NP epitope efficiently. Cells infected with DP-L2851 were able to present the NP epitope, showing that the inability of DP-L2840 to present the NP epitope under the experimental conditions can be attributed to inefficient escape from the vacuole. The increased efficiency of DP-L2028 over DP-L2028 under the conditions utilized was likely due to the presence of a multicopy plasmid in DP-L2028, whereas DP-L2851 expresses the NP epitope from a single copy gene in the chromosome. Another possible explanation is the absence of CD4⁺ T cell epitopes in DP-L2028, which contains only the dominant CD8⁺ T cell epitope of NP.

To determine the immunogenicity of DP-L2028 and DP-L2851 in vivo, splenocytes were isolated from immunized BALB/c mice and stimulated in vitro with the K^{d}-restricted NP peptide. Both recombinant strains of LM were able to induce NP-specific CTL, as evidenced by cytolysis of peptide-pulsed and influenza-infected targets. In addition, when mice were challenged 3 weeks post-vaccination with a sublethal dose of A/PR/8/34 virus, both DP-L2028 and DP-L2851 afforded statistically significant reductions (0.5-0.7 log) in the lung viral titers compared to naive mice or mice immunized with wild-type LM.

Thus, immunogenicity is enhanced in constructs in which the target epitope is either fused to, or embedded within, an immunogenic peptide.

### Example 9

### ActA-Antigen and PEST-Antigen Fusions Confer Anti-Tumor Immunity

### Materials and Experimental Methods

### Construction of Lm-ActA-E7

Lm-ActA-E7 is a recombinant strain of LM, comprising a plasmid that expresses the E7 protein fused to a truncated version of the actA protein. Lm-actA-E7 was generated by introducing a plasmid vector pDD-1, constructed by modifying pDP-2028, into Listeria. pDD-1 comprises an expression cassette expressing a copy of the 310 bp hly promoter and the hly signal sequence (ss), which drives the expression and secretion of ActA-E7; 1170 bp of the actA gene that comprises four PEST sequences (SEQ ID NO: 21) (the truncated ActA polypeptide consists of the first 390 AA of the molecule, SEQ ID NO: 20); the 300 bp HPV E7 gene; the 1019 bp prfA gene (controls expression of the virulence genes); and the CAT gene (chloramphenicol resistance gene) for selection of transformed bacteria clones (Sewell et al. (2004), Arch. Otolaryngol. Head Neck Surg., 130: 92-97).

The hly promoter (pHly) and gene fragment were PCR amplified from pGG55 (Example 1) using primer 5′-GGGGTCTAGACCTCCTTTGATTAGTATATTC-3′ (Xba I site is underlined; SEQ ID NO: 61) and primer 5′-ATCTTCGCTATCTGTCGCCGCGGCGCGTGCTTCAGTTTGTTGCGC-′3 (Not I site is underlined. The first 18 nucleotides are the ActA gene overlap; SEQ ID NO: 62). The actA gene was PCR amplified from the LM 10403s wildtype genome using primer 5′-GCGCAACAAACTGAAGCAGCGGCCGCGGCGACAGATAGCGAAGAT-3′ (NotI site is underlined; SEQ ID NO: 63) and primer 5′-TGTAGGTGTATCTCCATGCTCGAGAGCTAGGCGATCAATTTC-3′ (XhoI site is underlined; SEQ ID NO: 64). The E7 gene was PCR amplified from pGG55 (pLLO-E7) using primer 5′-GGAATTGATCGCCTAGCTCTCGAGCATGGAGATACACCTACA-3′ (XhoI site is underlined; SEQ ID NO: 65) and primer 5′-AAACGGATTTATTTAGATCCCGGGTTATGGTTTCTGAGAACA-3′ (XmaI site is underlined; SEQ ID NO: 66). The prfA gene was PCR amplified from the LM 10403s wild-type genome using primer 5′-TGTTCTCAGAAACCATAACCCGGGATCTAAATAAATCCGTTT-3′ (XmaI site is underlined; SEQ ID NO: 67) and primer 5′-GGGGGTCGACCAGCTCTTCTTGGTGAAG-3′ (SalI site is underlined; SEQ ID NO: 24). The hly promoter-actA gene fusion (pHly-actA) was PCR generated and amplified from purified pHly DNA and purified actA DNA using the upstream pHly primer (SEQ ID NO: 61) and downstream actA primer (SEQ ID NO: 64).

The E7 gene fused to the prfA gene (E7-prfA) was PCR generated and amplified from purified E7 DNA and purified prfA DNA using the upstream E7 primer (SEQ ID NO: 65) and downstream prfA gene primer (SEQ ID NO: 24).

The pHly-actA fusion product fused to the E7-prfA fusion product was PCR generated and amplified from purified fused pHly-actA DNA product and purified fused E7-prfA DNA product using the upstream pHly primer (SEQ ID NO: 61) and downstream prfA gene primer (SEQ ID NO: 24) and ligated into pCRII (Invitrogen, La Jolla, Calif.). Competent E. coli (TOP10′F, Invitrogen, La Jolla, Calif.) were transformed with pCRII-ActAE7. After lysis and isolation, the plasmid was screened by restriction analysis using BamHI (expected fragment sizes 770 bp and 6400 bp (or when the insert was reversed into the vector: 2500 bp and 4100 bp)) and BstXI (expected fragment sizes 2800 bp and 3900 bp) and also screened with PCR analysis using the upstream pHly primer (SEQ ID NO: 61) and the downstream prfA gene primer (SEQ ID NO: 24).

The pHly-actA-E7-prfA DNA insert was excised from pCRII by double digestion with Xba land Sal I and ligated into pDP-2028 also digested with Xba I and Sal I. After transforming TOP10′F competent E. coli (Invitrogen, La Jolla, Calif.) with expression system pActAE7, chloramphenicol resistant clones were screened by PCR analysis using the upstream pHly primer (SEQ ID NO: 61) and the downstream PrfA gene primer (SEQ ID NO: 24). A clone comprising pActAE7 was grown in brain heart infusion medium (with chloramphenicol (20 mcg (microgram)/ml (milliliter), Difco, Detroit, Mich.) and pActAE7 was isolated from the bacteria cell using a midiprep DNA purification system kit (Promega, Madison, Wis.). A prfA-negative strain of penicillin-treated Listeria (strain XFL-7) was transformed with expression system pActAE7, as described in Ikonomidis et al. (1994, J. Exp. Med. 180: 2209-2218) and clones were selected for the retention of the plasmid in vivo. Clones were grown in brain heart infusion with chloramphenicol (20 mcg/ml) at 37° C. Bacteria were frozen in aliquots at −80° C.

### Immunoblot Verification of Antigen Expression

To verify that Lm-ActA-E7 secretes ActA-E7, (about 64 kD), Listeria strains were grown in Luria-Bertoni (LB) medium at 37° C. Protein was precipitated from the culture supernatant with trichloroacetic acid (TCA) and resuspended in 1× sample buffer with 0.1N sodium hydroxide. Identical amounts of each TCA precipitated supernatant were loaded on 4% to 20% Tris-glycine sodium dodecyl sulfate-polyacrylamide gels (NOVEX, San Diego, Calif.). Gels were transferred to polyvinylidene difluoride membranes and probed with 1:2500 anti-E7 monoclonal antibody (Zymed Laboratories, South San Francisco, Calif.), then with 1:5000 horseradish peroxidase-conjugated anti-mouse IgG (Amersham Pharmacia Biotech, Little Chalfont, England). Blots were developed with Amersham enhanced chemiluminescence detection reagents and exposed to autoradiography film (Amersham) (FIG. 9A).

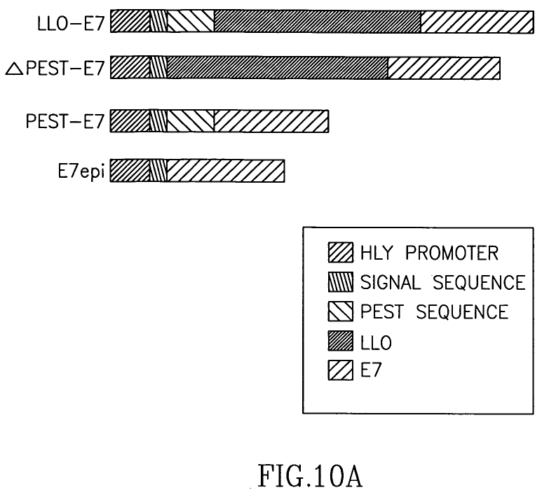

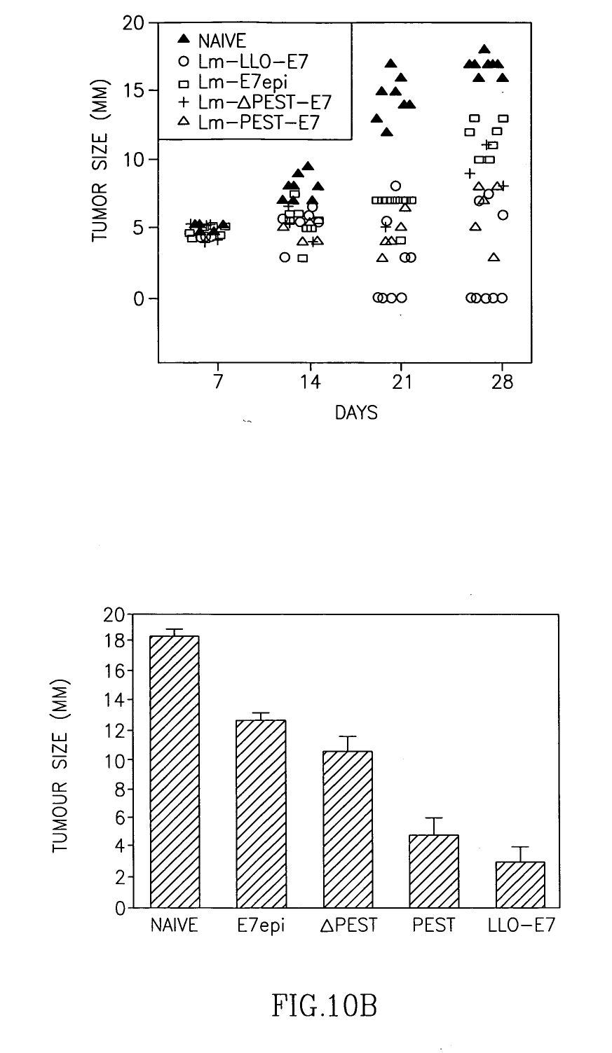

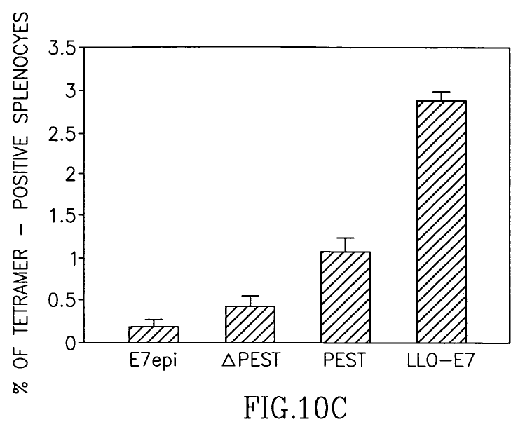

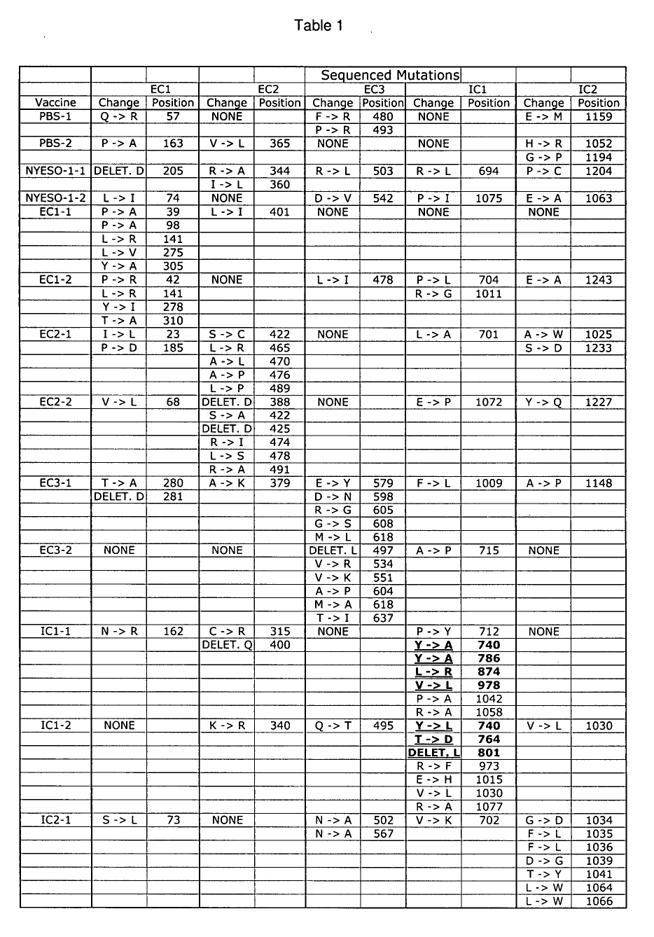

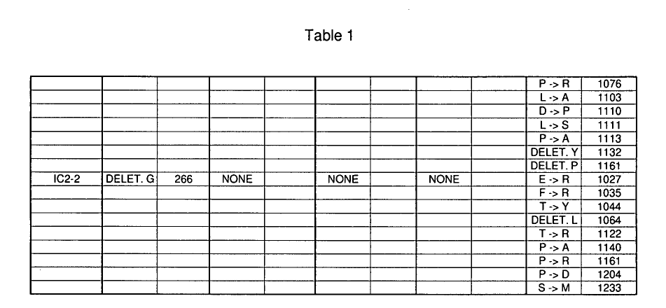

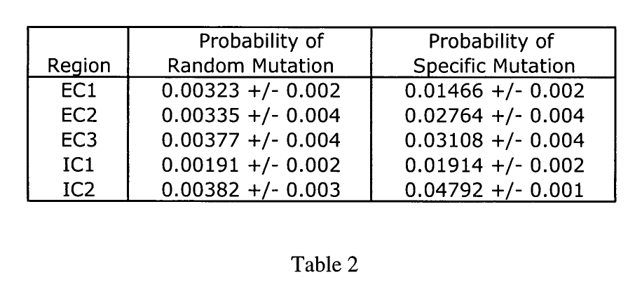

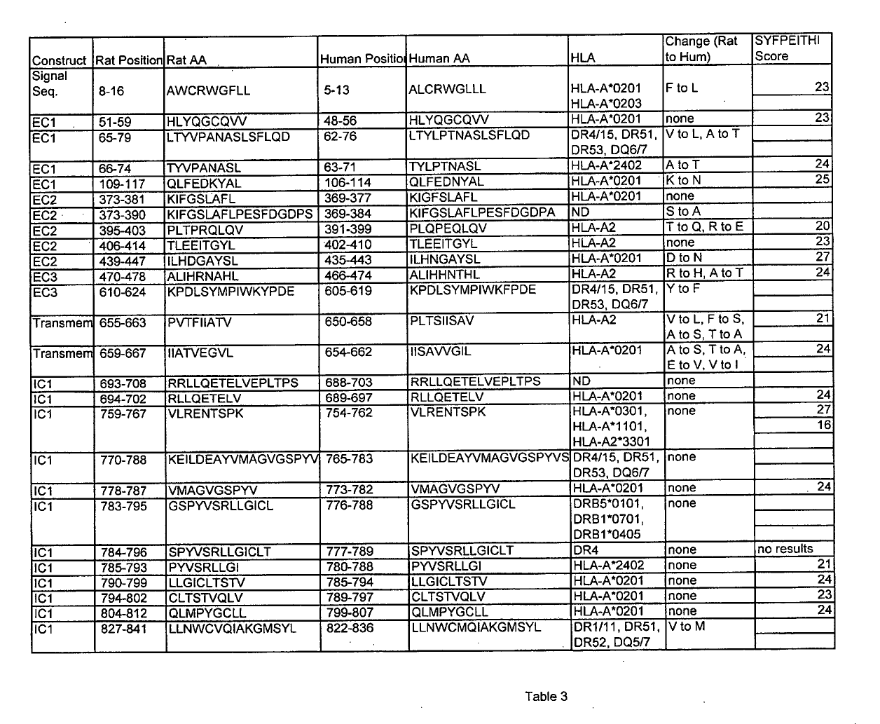

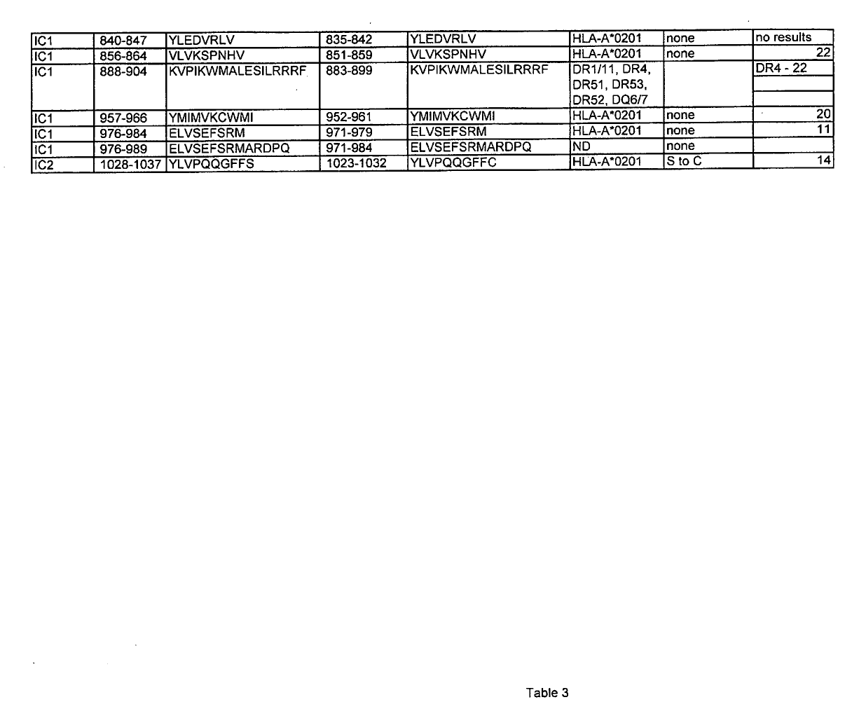

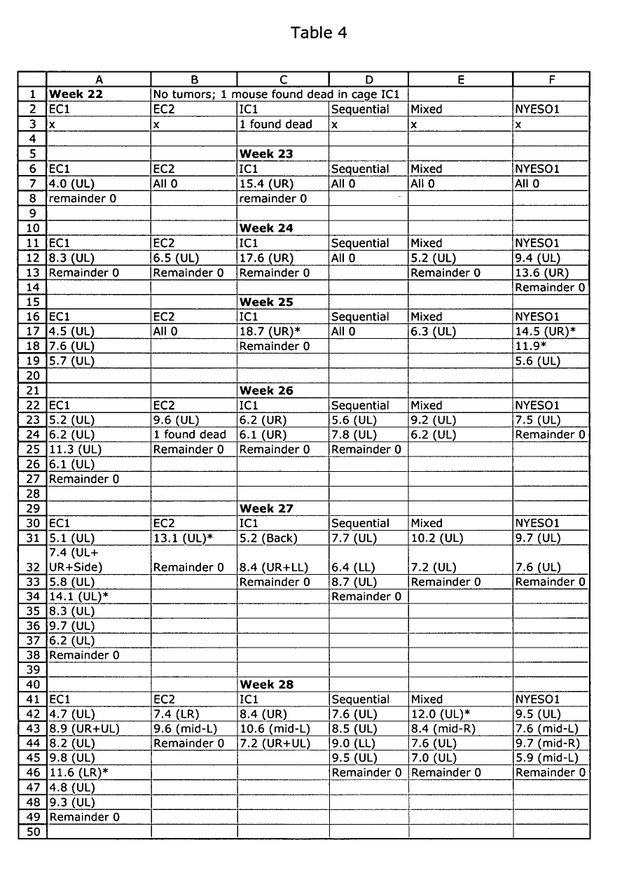

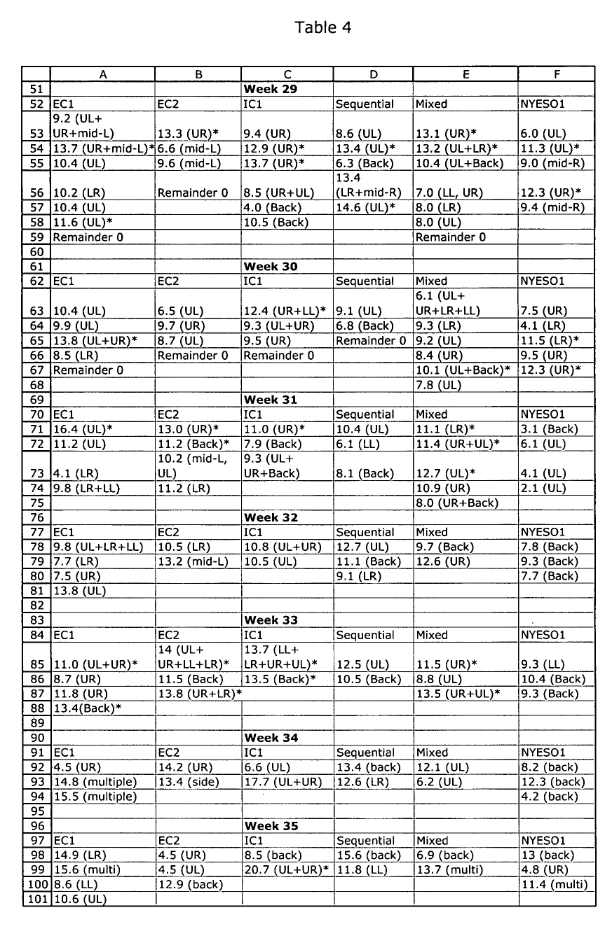

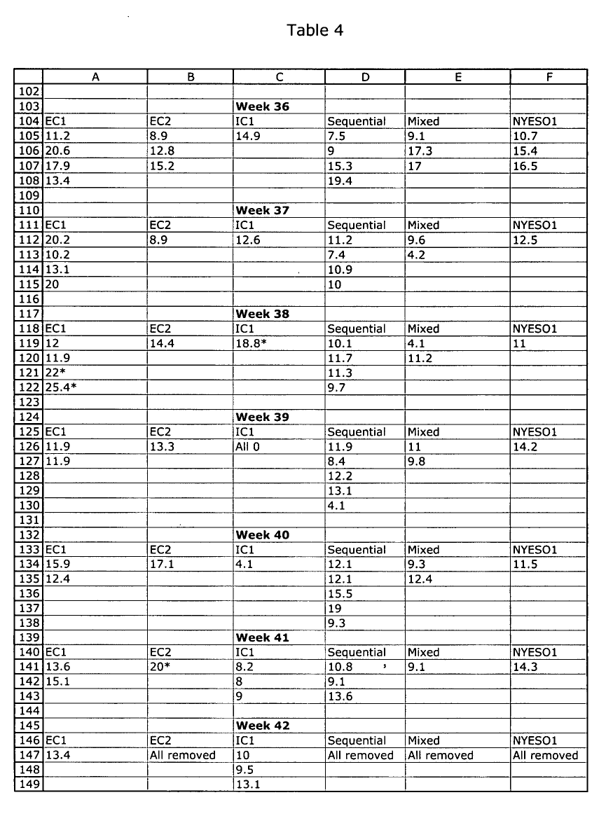

### Construction of Lm-PEST-E7, Lm-ΔPEST-E7, and Lm-E7epi (FIG. 10A)

Lm-PEST-E7 is identical to Lm-LLO-E7, except that it contains only the promoter and PEST sequence of the hly gene, specifically the first 50 AA of LLO. To construct Lm-PEST-E7, the hly promoter and PEST regions were fused to the full-length E7 gene using the SOE (gene splicing by overlap extension) PCR technique. The E7 gene and the hly-PEST gene fragment were amplified from the plasmid pGG-55, which contains the first 441 AA of LLO, and spliced together by conventional PCR techniques. To create a final plasmid, pVS16.5, the hly-PEST-E7 fragment and the prfA gene were subcloned into the plasmid pAM401, which includes a chloramphenicol resistance gene for selection in vitro, and the resultant plasmid was used to transform XFL-7.

Lm-ΔPEST-E7 is a recombinant Listeria strain that is identical to Lm-LLO-E7 except that it lacks the PEST sequence. It was made essentially as described for Lm-PEST-E7, except that the episomal expression system was constructed using primers designed to remove the PEST-containing region (bp 333-387) from the hly-E7 fusion gene. Lm-E7epi is a recombinant strain that secretes E7 without the PEST region or LLO. The plasmid used to transform this strain contains a gene fragment of the hly promoter and signal sequence fused to the E7 gene. This construct differs from the original Lm-E7, which expressed a single copy of the E7 gene integrated into the chromosome. Lm-E7epi is completely isogenic to Lm-LLO-E7, Lm-PEST-E7, and Lm-ΔPEST-E7 except for the form of the E7 antigen expressed.

### Results

To compare the anti-tumor immunity induced by Lm-ActA-E7 versus Lm-LLO-E7, 2×10⁵ TC-1 tumor cells were implanted subcutaneously in mice and allowed to grow to a palpable size (approximately 5 millimeters [mm]). Mice were immunized i.p. with one LD₅₀ of either Lm-ActA-E7 (5×10⁸ CFU), (crosses) Lm-LLO-E7 (10⁸ CFU) (squares) or Lm-E7 (10⁶ CFU) (circles) on days 7 and 14. By day 26, all of the animals in the Lm-LLO-E7 and Lm-ActA-E7 were tumor free and remained so, whereas all of the naive animals (triangles) and the animals immunized with Lm-E7 grew large tumors (FIG. 9B). Thus, vaccination with ActA-E7 fusions induces tumor regression.

In addition, Lm-LLO-E7, Lm-PEST-E7, Lm-ΔPEST-E7, and Lm-E7epi were compared for their ability to cause regression of E7-expressing tumors. S.c. TC-1 tumors were established on the left flank of 40 C57BL/6 mice. After tumors had reached 4-5 mm, mice were divided into 5 groups of 8 mice. Each groups was treated with 1 of 4 recombinant LM vaccines, and 1 group was left untreated. Lm-LLO-E7 and Lm-PEST-E7 induced regression of established tumors in 5/8 and 3/8 cases, respectively. There was no statistical difference between the average tumor size of mice treated with Lm-PEST-E7 or Lm-LLO-E7 at any time point. However, the vaccines that expressed E7 without the PEST sequences, Lm-ΔPEST-E7 and Lm-E7epi, failed to cause tumor regression in all mice except one (FIG. 10B, top panel). This was representative of 2 experiments, wherein a statistically significant difference in mean tumor sizes at day 28 was observed between tumors treated with Lm-LLO-E7 or Lm-PEST-E7 and those treated with Lm-E7epi or Lm-ΔPEST-E7; P<0.001, Student's t test; FIG. 10B, bottom panel). In addition, increased percentages of tetramer-positive splenocytes were seen reproducibly over 3 experiments in the spleens of mice vaccinated with PEST-containing vaccines (FIG. 10C). Thus, vaccination with PEST-antigen fusions causes tumor regression.

1. A method of inducing an immune response in a subject against a protein antigen of interest, the method comprising the steps of:
A. administering to said subject a vaccine comprising a first recombinant peptide comprising a first fragment of said protein antigen of interest; and
B. after the conclusion of step (A), administering to said subject a vaccine comprising a second recombinant peptide, said second recombinant peptide comprising a second fragment of said protein antigen of interest,
thereby inducing an immune response in a subject against a protein antigen of interest.

2-3. (canceled)

4. The method of claim 1, wherein said first fragment or said second fragment comprises or overlaps a domain that mediates an oncogenic function of said tumor-associated protein.

5. (canceled)

6. The method of claim 1, wherein said first recombinant peptide and said second recombinant peptide further comprise a non-hemolytic LLO peptide, a non-hemolytic N-terminal fragment of an LLO peptide, an ActA peptide, or a PEST-containing sequence.

7-11. (canceled)

12. The method of claim 1, wherein said protein antigen of interest is a tumor-associated protein, an endogenous growth factor, or a vaso-active protein or peptide.

13-14. (canceled)

15. The method of claim 1, further comprising the step of, after the conclusion of step (B), administering to said subject a vaccine comprising a third recombinant peptide, said third recombinant peptide comprising a third fragment of said tumor-associated protein.

16. (canceled)

17. The method of claim 1, wherein at least one of said first recombinant peptide and said second recombinant peptide is expressed by a recombinant Listeria strain.

18. A method of treating or suppressing the formation of a tumor expressing a tumor-associated protein in a subject, comprising the steps of:
A. administering to said subject a vaccine comprising a first recombinant peptide comprising a first fragment of said tumor-associated protein; and
B. subsequent to step (A), administering to said subject a vaccine comprising a second recombinant peptide, said second recombinant peptide comprising a second fragment of said tumor-associated protein.

19. The method of claim 18, wherein said tumor-associated protein is a kinase.

20. (canceled)

21. The method of claim 18, wherein said first fragment or said second fragment comprises or overlaps a domain that mediates an oncogenic function of said tumor-associated protein.

22. (canceled)

23. The method of claim 18, wherein said first recombinant peptide and said second recombinant peptide further comprise a non-hemolytic LLO peptide, a non-hemolytic N-terminal fragment of an LLO peptide, an ActA peptide or a PEST-containing sequence.

24-28. (canceled)

29. The method of claim 18, further comprising the step of, subsequent to step (B), administering to said subject a vaccine comprising a third recombinant peptide, said third recombinant peptide comprising a third fragment of said tumor-associated protein.

30. (canceled)

31. The method of claim 18, wherein at least one of said first recombinant peptide and said second recombinant peptide is expressed by a recombinant Listeria strain.

32-45. (canceled)

46. A method of treating or suppressing the formation of a tumor expressing a Her-2 protein in a subject, comprising the steps of:
A. administering to said subject a first recombinant peptide comprising a first fragment of said Her-2 protein, wherein said first fragment is selected from:
1. a first intracellular fragment of said Her-2 protein, comprising the kinase domain of said Her-2 protein or a fragment of said kinase domain;
2. a second intracellular fragment of said Her-2 protein, wherein said second intracellular fragment either does not overlap with said first intracellular fragment or overlaps less than 20% with said first intracellular fragment;
3. a first extracellular fragment of said Her-2 protein, comprising the N-terminal half of the extracellular domain of said Her-2 protein or a fragment of said N-terminal half;
4. a second extracellular fragment of said Her-2 protein, comprising the middle third of the extracellular domain of said Her-2 protein or a fragment of said middle third; and
B. subsequent to step (A), administering to said subject a second recombinant peptide, said second recombinant peptide comprising a second fragment of said Her-2 protein, wherein said second fragment is selected from above fragments 1-4, but different from said first fragment.

47. The method of claim 46, wherein said first recombinant peptide and said second recombinant peptide further comprise a non-hemolytic LLO peptide, a non-hemolytic N-terminal fragment of an LLO peptide, an ActA peptide or a PEST-containing sequence.

48-52. (canceled)

53. The method of claim 46, further comprising the step of, subsequent to step (B), administering to said subject a vaccine comprising a third recombinant peptide, said third recombinant peptide comprising a third fragment of said Her-2 protein, wherein said third fragment is selected from above fragments 1-4, but is different from said first fragment and said second fragment.

54. (canceled)

55. The method of claim 46, wherein at least one of said first recombinant peptide and said second recombinant peptide is expressed by a recombinant Listeria strain.

56-61. (canceled)

62. A recombinant peptide comprising a fragment of a Her-2 protein, wherein said fragment is selected from GSGAFGTVYK (SEQ ID No: 68), AFGTVYKGI (SEQ ID No: 69), PYVSRLLGI (SEQ ID No: 70), TSPKANKEI (SEQ ID No. 71), RPRFRELVSE (SEQ ID No: 72), STVQLVTQL (SEQ ID No: 73), and KITDFGLARL (SEQ ID No: 74).

63. The recombinant peptide of claim 62, wherein said recombinant peptide further comprises a non-hemolytic LLO peptide.

64. A vaccine comprising the recombinant peptide of claim 62 and an adjuvant.

65-67. (canceled)

68. A nucleotide molecule encoding the recombinant peptide of claim 62.

69. (canceled)

70. A recombinant vaccine vector comprising the nucleotide molecule of claim 68.

71-74. (canceled)

75. A method of treating or reducing an incidence of a Her-2-expressing tumor, or eliciting an anti-Her-2 immune response in a subject, comprising administering to said subject the recombinant peptide of claim 62.

76. A method of treating or reducing an incidence of a Her-2-expressing tumor, or eliciting an anti-Her-2 immune response in a subject, comprising administering to said subject the nucleotide molecule of claim 68.

77-78. (canceled)

79. The method of claim 17, wherein said recombinant Listeria strain has been passaged through an animal host.

80. The method of claim 31, wherein said recombinant Listeria strain has been passaged through an animal host.

81. The method of claim 55, wherein said recombinant Listeria strain has been passaged through an animal host.
